(19) United States
(12) Patent Application Publication
(10) Pub. No.: US 2022/0403323 A1
(43) Pub. Date: Dec. 22, 2022

(54) TEXTURING L. LACTIS WITH UNIQUE EPS GENE CLUSTERS

(71) Applicant: Chr. Hansen A/S, Hoersholm (DK)

(72) Inventors: Vera Kuzina POULSEN, Hoersholm (DK); Gunnar OEREGAARD, Hoersholm (DK); Elahe Ghanei MOGHADAM, Hoersholm (DK)

(73) Assignee: Chr. Hansen A/S, Hoersholm (DK)

(21) Appl. No.: 17/634,909

(22) PCT Filed: Aug. 21, 2020
(86) PCT No.: PCT/EP2020/073522

§ 371 Date: Feb. 11, 2022

(30) Foreign Application Priority Data
Aug. 23, 2019 (EP) 19193295.3
Aug. 23, 2019 (EP) 19193299.5
Aug. 23, 2019 (EP) 19193303.5
Aug. 23, 2019 (EP) 19193305.0
Aug. 23, 2019 (EP) 19193307.6
Aug. 23, 2019 (EP) 19193308.4
Aug. 23, 2019 (EP) 19193310.0
Aug. 23, 2019 (EP) 19193312.6
Aug. 23, 2019 (EP) 19193313.4
Aug. 23, 2019 (EP) 19193315.9
Aug. 23, 2019 (EP) 19193316.7

(51) Int. Cl. C12N 1/20; A23C 11/10; C12N 1/04; A23C 9/123; C12N 9/10; C12N 15/52; C12N 9/12; C12N 9/14

(52) U.S. Cl. CPC C12N 1/205; A23C 11/106; C12N 1/04; A23C 9/1234; C12N 9/1048; C12N 15/52; C12N 9/1241; C12N 9/14; C12R 2001/225

(57) ABSTRACT

The present invention provides novel Lactococcus lactis lactic acid bacterium strains having improved texturing properties and methods of using the strains for producing food products.

### CROSS-REFERENCE TO RELATED APPLICATIONS

The present application is the U.S. National Stage of International Application No. PCT/EP2020/073522, filed Aug. 21, 2020, and claims priority to European Patent Application Nos. 19193295.3, 19193299.5, 19193303.5, 19193305.0, 19193307.6, 19193308.4, 19193310.0, 19193312.6, 19193313.4, 19193315.9, and 19193316.7, all filed Aug. 23, 2019.

### FIELD OF THE INVENTION

The present invention relates to novel Lactococcus lactic lactic acid bacterium (LAB) strains, having improved texturing properties. The present invention also relates to methods of using the strains for making food products and to food products comprising the strains.

### BACKGROUND OF THE INVENTION

Lactic acid bacteria (LAB) are used extensively by the food industry for fermentation of food. Conversion of fresh milk to fermented milk by LAB is a way of extending the life time of the milk and provides taste as well as texture.

Thus, important features of the strains used for milk fermentation include fast acidification, stable (no/low) post-acidification, long shelf-life and good texture. Good texture is typically high mouth thickness and viscosity (measured as high shear stress using a rheometer) and high gel firmness.

Some LAB strains contribute significantly to an improved texture associated with their ability to produce exo- (or extracellular) polysaccharides (EPS), which can be capsular (remain attached to the cell in the form of capsules) or secreted into the media. EPS consists of either a single type of sugar (homo-exopolysaccharides) or repeating units made of different sugars (hetero-exopolysaccharides). EPS-producing LAB are of interest, since EPS act as natural viscosifiers and texture enhancers of fermented foods. Furthermore, EPS from food-grade LAB with defined rheological properties have potential for development and exploitation as food additives. EPS are known to improve the rheological properties of LAB-fermented products by influencing viscosity, syneresis, firmness and sensory properties. The primary structural features (monosaccharide type and configuration, glycosidic linkage, non-sugar decorations, charge), the conformation and molecular weight, the amount of polysaccharide and the interactions of the polysaccharide with other system components are all factors that can contribute to and influence the displayed techno-functional properties (Zeidan et al., 2017).

Fermented milk can be produced by mesophilic LAB, e.g. Lactococcus sp. leading to, e.g., sour milk, or thermophilic LAB, e.g., Streptococcus thermophilus and Lactobacillus delbruckii subsp. bulgaricus, for yoghurt. Dairy products, such as fresh cheese, butter milk, sour milk and sour cream, prepared with mesophilic starter cultures, such as Lactococcus lactis, are in popular demand with consumers. In addition, market for dairy alternative products, where plant bases fermented with L. lactis can play a role, is growing. Consumers with lactose intolerance and milk allergy, as well as consumers with concerns about cow milk hormones and cholesterol, animal well-being and impact of animal-based food on the environment play a role in the increasing demand. Also, plant-based diet is supposedly healthier than meat-based diet (Tangyu et al., 2019).

Several texturing L. lactis strains have been reported, e.g. NIZO B40, SMQ-461, Ropy352, JFR1, LIi3, LII8 (for a review see Poulsen et al., 2019). The structure of EPS of NIZO B40 has been elucidated and the functional characterization of genes responsible for the EPS biosynthesis has been performed (Kleerebezem et al., 2002).

Pan and Mei (2010) characterized EPS produced by L. lactis subsp. lactis, which was isolated from Chinese pickled cabbage, but it is not known if this strain is able to acidify milk and contribute to its texture. No eps genes were reported for this strain (Pan and Mei, 2010).

Suzuki et al. (2013) reported the sequences of a highly conserved epsD gene and a strain-specific epsE gene in five lactococcal strains, two from the subsp. lactis biovar diacetylactis and two from the subsp. cremoris. However, neither information on a complete eps gene cluster nor if the EPS produced by these strains is able to enhance milk texture is available.

WO 2017/108679 relates to the novel strain Lactococcus lactis subsp. lactis DSM 29291, which had the highest shear stress out of the eight different L. lactis subsp. lactis strains tested, both according to the TADM and the rheometer measurements (see Example 1 and FIG. 1 of WO 2017/108679).

Since mesophilic cultures are used for fermented milk products, and texture is an important parameter, there is a need for further texturing mesophilic strains, in particular for improved texturing mesophilic strains, e.g., texturing Lactococcus lactis strains.

### SUMMARY OF THE INVENTION

In a first aspect, the present invention relates to a Lactococcus lactis lactic acid bacterium strain comprising an active eps gene cluster capable of producing exopolysaccharide (EPS), wherein the eps gene cluster comprises the below nucleotide sequences ((a), (b) and (c), as the case may be) as defined in any one of (i) to (x):
(i) (a): a nucleotide sequence encoding a polypeptide having polymerase activity and having at least 95% identity with the amino acid sequence encoded by the nucleotide sequence of SEQ ID NO: 11 (herein termed wzy);
   (b): a nucleotide sequence encoding a polypeptide having polysaccharide transporter activity and having at least 95% identity with the amino acid sequence encoded by the nucleotide sequence of SEQ ID NO: 17 (herein termed wzx); and
   (c): a nucleotide sequence encoding a polypeptide having glycosyltransferase (GT) activity comprising:
   (c1): a nucleotide sequence having at least 95% identity with the amino acid sequence encoded by the nucleotide sequence of SEQ ID NO: 9 (herein termed GT1);
   (c2): a nucleotide sequence having at least 95% identity with the amino acid sequence encoded by the nucleotide sequence of SEQ ID NO: 13 (herein termed GT2); and
   (c3): a nucleotide sequence having at least 95% identity with the amino acid sequence encoded by the nucleotide sequence of SEQ ID NO: 15 (herein termed GT3);
(ii) (a): a nucleotide sequence encoding a polypeptide having polymerase activity and having at least 95% identity with the amino acid sequence encoded by nucleotides 13939-15042 of SEQ ID NO:199 (herein termed wzy);
   (b): a nucleotide sequence encoding a polypeptide having polysaccharide transporter activity and having at least 95% identity with the amino acid sequence encoded by nucleotides 26029-27444 of the complementary strand of SEQ ID NO:199 (herein termed wzx); and
   (c): a nucleotide sequence encoding a polypeptide having glycosyltransferase (GT) activity comprising:
   (c1): a nucleotide sequence having at least 95% identity with the amino acid sequence encoded by nucleotides 4174-4824 of SEQ ID NO:199 (herein termed GT1);
   (c2): a nucleotide sequence having at least 95% identity with the amino acid sequence encoded by nucleotides 7276-8508 of SEQ ID NO:199 (herein termed GT2);
   (c3): a nucleotide sequence having at least 95% identity with the amino acid sequence encoded by nucleotides 11042-12391 of SEQ ID NO:199 (herein termed GT3);
   (c4): a nucleotide sequence having at least 95% identity with the amino acid sequence encoded by nucleotides 13008-13934 of SEQ ID NO:199 (herein termed GT4); and
   (c5): a nucleotide sequence having at least 95% identity with the amino acid sequence encoded by nucleotides 18528-19508 of SEQ ID NO:199 (herein termed GT5);
(iii) (a): a nucleotide sequence encoding a polypeptide having polymerase activity and having at least 95% identity with the amino acid sequence encoded by the nucleotide sequence of SEQ ID NO: 39 (herein termed wzy);
   (b): a nucleotide sequence encoding a polypeptide having polysaccharide transporter activity and having at least 95% identity with the amino acid sequence encoded by the nucleotide sequence of SEQ ID NO: 45 (herein termed wzx);
   (c): a nucleotide sequence encoding a polypeptide having glycosyltransferase (GT) activity comprising:
   (c1): a nucleotide sequence having at least 95% identity with the amino acid sequence encoded by the nucleotide sequence of SEQ ID NO: 37 (herein termed GT1);
   (c2): a nucleotide sequence having at least 95% identity with the amino acid sequence encoded by the nucleotide sequence of SEQ ID NO: 41 (herein termed GT2); and
   (c3): a nucleotide sequence having at least 95% identity with the amino acid sequence encoded by the nucleotide sequence of SEQ ID NO: 43 (herein termed GT3);
(iv) (a): a nucleotide sequence encoding a polypeptide having polymerase activity and having at least 95% identity with the amino acid sequence encoded by the nucleotide sequence of SEQ ID NO: 163 (herein termed wzy);
   (b): a nucleotide sequence encoding a polypeptide having polysaccharide transporter activity and having at least 95% identity with the amino acid sequence encoded by the nucleotide sequence of SEQ ID NO: 169 (herein termed wzx); and
   (c): a nucleotide sequence encoding a polypeptide having glycosyltransferase (GT) activity comprising:
   (c1): a nucleotide sequence having at least 95% identity with the amino acid sequence encoded by the nucleotide sequence of SEQ ID NO: 161 (herein termed GT1);
   (c2): a nucleotide sequence having at least 95% identity with the amino acid sequence encoded by the nucleotide sequence of SEQ ID NO: 165 (herein termed GT2);
   (c3): a nucleotide sequence having at least 95% identity with the amino acid sequence encoded by the nucleotide sequence of SEQ ID NO: 167 (herein termed GT3); and
   (c4) a nucleotide sequence having at least 95% identity with the amino acid sequence encoded by the nucleotide sequence of SEQ ID NO: 181 (herein termed GT4);
(v) (a): a nucleotide sequence encoding a polypeptide having polymerase activity and having at least 95% identity with the amino acid sequence encoded by nucleotides 13939-15042 of SEQ ID NO: 224 (herein termed wzy); and
   (c): a nucleotide sequence encoding a polypeptide having glycosyltransferase (GT) activity comprising:
   (c1): a nucleotide sequence having at least 95% identity with the amino acid sequence encoded by nucleotides 4174-4824 of SEQ ID NO: 224 (herein termed GT1);
   (c2): a nucleotide sequence having at least 95% identity with the amino acid sequence encoded by nucleotides 11042-12391 of SEQ ID NO: 224 (herein termed GT2);
   (c3): a nucleotide sequence having at least 95% identity with the amino acid sequence encoded by nucleotides 13008-13934 of SEQ ID NO: 224 (herein termed GT3); and
   (c4): a nucleotide sequence having at least 95% identity with the amino acid sequence encoded by nucleotides 18527-19507 of SEQ ID NO: 224 (herein termed GT4);
(vi) (a): a nucleotide sequence encoding a polypeptide having polymerase activity and having at least 95% identity with the amino acid sequence encoded by the nucleotide sequence of SEQ ID NO: 67 (herein termed wzy);
   (b): a nucleotide sequence encoding a polypeptide having polysaccharide transporter activity and having at least 95% identity with the amino acid sequence encoded by the nucleotide sequence of SEQ ID NO: 73 (herein termed wzx);
   (c): a nucleotide sequence encoding a polypeptide having glycosyltransferase (GT) activity comprising:
   (c1): a nucleotide sequence having at least 95% identity with the amino acid sequence encoded by the nucleotide sequence of SEQ ID NO: 65 (herein termed GT1);
   (c2): a nucleotide sequence having at least 95% identity with the amino acid sequence encoded by the nucleotide sequence of SEQ ID NO: 69 (herein termed GT2); and
   (c3): a nucleotide sequence having at least 95% identity with the amino acid sequence encoded by the nucleotide sequence of SEQ ID NO: 71 (herein termed GT3);
   (c4): a nucleotide sequence having at least 95% identity with the amino acid sequence encoded by the nucleotide sequence of SEQ ID NO: 85 (herein termed GT4);
   (c5): a nucleotide sequence having at least 95% identity with the amino acid sequence encoded by the nucleotide sequence of SEQ ID NO: 87 (herein termed GT5); and
   (c6): a nucleotide sequence having at least 95% identity with the amino acid sequence encoded by the nucleotide sequence of SEQ ID NO: 89 (herein termed GT6);
(vii) (a): a nucleotide sequence encoding a polypeptide having polymerase activity and having at least 95% identity with the amino acid sequence encoded by nucleotides 5833-6927 of SEQ ID NO: 244 (herein termed wzy); and
   (c): a nucleotide sequence encoding a polypeptide having glycosyltransferase (GT) activity comprising:
   (c1): a nucleotide sequence having at least 95% identity with the amino acid sequence encoded by nucleotides 4617-5123 of SEQ ID NO: 244 (herein termed GT1); and
   (c2): a nucleotide sequence having at least 95% identity with the amino acid sequence encoded by nucleotides 5120-5827 of SEQ ID NO: 244 (herein termed GT2);
(viii) (a): a nucleotide sequence encoding a polypeptide having polymerase activity and having at least 95% identity with the amino acid sequence encoded by the nucleotide sequence of SEQ ID NO: 123 (herein termed wzy);
   (b): a nucleotide sequence encoding a polypeptide having polysaccharide transporter activity and having at least 95% identity with the amino acid sequence encoded by the nucleotide sequence of SEQ ID NO: 129 (herein termed wzx); and
   (c): a nucleotide sequence encoding a polypeptide having glycosyltransferase (GT) activity comprising:
   (c1): a nucleotide sequence having at least 95% identity with the amino acid sequence encoded by the nucleotide sequence of SEQ ID NO: 121 (herein termed GT1);
   (c2): a nucleotide sequence having at least 95% identity with the amino acid sequence encoded by the nucleotide sequence of SEQ ID NO: 125 (herein termed GT2);
   (c3): a nucleotide sequence having at least 95% identity with the amino acid sequence encoded by the nucleotide sequence of SEQ ID NO: 127 (herein termed GT3);
   (c4): a nucleotide sequence having at least 95% identity with the amino acid sequence encoded by the nucleotide sequence of SEQ ID NO: 143 (herein termed GT4); and
   (c5): a nucleotide sequence having at least 95% identity with the amino acid sequence encoded by the nucleotide sequence of SEQ ID NO: 145 (herein termed GT5); and
   (c6): a nucleotide sequence having at least 95% identity with the amino acid sequence encoded by the nucleotide sequence of SEQ ID NO: 147 (herein termed GT6);
(ix) (a): a nucleotide sequence encoding a polypeptide having polymerase activity and having at least 95% identity with the amino acid sequence encoded by nucleotides 11201-12349 of the complementary strand of SEQ ID NO: 257 (herein termed wzy);
   (b): a nucleotide sequence encoding a polypeptide having polysaccharide transporter activity and having at least 95% identity with the amino acid sequence encoded by nucleotides 15538-16953 of the complementary strand of SEQ ID NO: 257 (herein termed wzx); and
   (c): a nucleotide sequence encoding a polypeptide having glycosyltransferase (GT) activity comprising:
   (c1): a nucleotide sequence having at least 95% identity with the amino acid sequence encoded by nucleotides 9726-10673 of the complementary strand of SEQ ID NO: 257 (herein termed GT1);
   (c2): a nucleotide sequence having at least 95% identity with the amino acid sequence encoded by nucleotides 12336-13421 of the complementary strand of SEQ ID NO: 257 (herein termed GT2); and
   (c3): a nucleotide sequence having at least 95% identity with the amino acid sequence encoded by nucleotides 13418-14260 of the complementary strand of SEQ ID NO: 257 (herein termed GT3);
(x) (a): a nucleotide sequence encoding a polypeptide having polymerase activity and having at least 95% identity with the amino acid sequence encoded by nucleotides 10707-11846 of the complementary strand of SEQ ID NO: 274 (herein termed wzy);
   (b): a nucleotide sequence encoding a polypeptide having polysaccharide transporter activity and having at least 95% identity with the amino acid sequence encoded by nucleotides 15037-16476 of the complementary strand of SEQ ID NO: 274 (herein termed wzx); and
   (c): a nucleotide sequence encoding a polypeptide having glycosyltransferase (GT) activity comprising:
   (c1): a nucleotide sequence having at least 95% identity with the amino acid sequence encoded by nucleotides 9232-10179 of the complementary strand of SEQ ID NO: 274 (herein termed GT1);
   (c2): a nucleotide sequence having at least 95% identity with the amino acid sequence encoded by nucleotides 11833-12918 of the complementary strand of SEQ ID NO: 274 (herein termed GT2); and
   (c3): a nucleotide sequence having at least 95% identity with the amino acid sequence encoded by nucleotides 12915-13757 of the complementary strand of SEQ ID NO: 274 (herein termed GT3);

The present invention further provides strain Lactococcus lactis subsp. lactis DSM 33192.

In a second aspect, the present invention relates to a composition comprising at least one texturing Lactococcus lactis lactic acid bacterium strain according to the present invention, as described above, preferably in combination with one or more further lactic acid bacterium strain(s), wherein the one or more further lactic acid bacterium strain(s) is(are) able to:
i) generate fermented milks with a target pH of about 4.55 in about 15 h or less, preferably in about 12 h or less, measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until a target pH of about 4.55 is reached. Therefore, the “time-to-pH 4.55” can be calculated for a certain lactic acid bacterium strain; and
ii) generate fermented milks having a shear stress of 40 Pa or more measured at shear rate 300 s⁻¹, measured under following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, time to pH 4.55) followed by storage at 4° C. until shear stress is measured, typically from 1-7 days, such as for 5 days, followed by gently stirring and measuring the shear stress at shear rate 300 s⁻¹, measured as described in the present application, wherein the inoculation temperature is 30° C.

In one embodiment the target pH may be e.g. pH between 4 and 5, preferably between pH 4.3 to 4.7, more preferably between pH 4.4 to 4.6, and even more preferably pH 4.45, pH 4.50, or pH 4.55.

Preferably, the composition according to the second aspect of the present invention comprises a texturing Lactococcus lactis lactic acid bacterium strain according to the present invention in combination with
(i) the lactic acid bacterium strain Lactococcus lactis subsp. cremoris DSM 25485, or a mutant or variant therefrom and/or
(ii) the lactic acid bacterium strain Lactococcus lactis subsp. lactis DSM 33192, or a mutant or variant therefrom and/or
(iii) the lactic acid bacterium strain Lactococcus lactis DSM 33133, or a mutant or variant therefrom.

The composition of the present invention may comprise further components, such as cryoprotectants, lyoprotectants, antioxidants, nutrients, fillers, flavorants or mixtures thereof, as described in detail below.

In a third aspect, the present invention relates to the use of the lactic acid bacterium strains (i) to (x) and/or the composition of the present invention for increasing the viscosity of a fermented milk product. The third aspect further relates to the use of the Lactococcus lactis subsp. cremoris strain DSM 25485 and/or the Lactococcus lactis subsp. lactis strain DSM 33192 for increasing viscosity (measured as shear stress with a shear rate of 300 s⁻¹, as described in the present invention) of a fermented milk product. The fermented milk product may be a mammalian-based fermented milk product (i.e., the milk base which is fermented has mammalian origin) or a plant-based fermented milk product (i.e., the milk base which is fermented is derived from plants, such as soy milk).

In a fourth aspect, the invention relates to a method of producing a food product comprising at least one stage in which at least one lactic acid bacterium strain as defined in the first aspect of the present invention, and/or the composition as defined in the second aspect of the present invention is used. Finally, the present invention relates to a food product comprising at least one lactic acid bacterium strain as defined in the first aspect of the present invention and/or the composition as defined in the second aspect of the present invention and/or strain DSM 33192. The food product may comprise further components, such as thickeners or stabilizers, or mixtures thereof, as described in detail below.

### BRIEF DESCRIPTION OF THE FIGURES

FIG. 1. Comparative analysis of eps gene clusters. A genetic comparison of a series of eps gene clusters of L. lactis available on the NCBI web-site and also those, which are the subject of this patent application, was performed, to assess the degree of similarity between different eps gene clusters. The analysis was based on fasta files containing the protein sequences encoded in eps gene clusters from 43 L. lactis strains. A subset of proteins (e.g. transposases) was excluded to ensure that the comparison was based only on functionally relevant proteins. The proteins were clustered using cd-hit (weizhongli-lab.org/cd-hit/) with an identity cutoff of 0.9, yielding 270 cd-hit groups from 712 protein sequences. A feature vector (length 270) was generated for each strain, based on presence or absence of proteins in each of the cd-hit groups. These feature vectors were used to compute the pairwise Jaccard similarities between all strains. The pairwise similarities were used to perform agglomerative hierarchical clustering. The clustering was done using the ‘single’ linkage method, as this results in a conservative clustering for assessing the novelty/uniqueness of the eps gene clusters, which are the subject of this patent application. With the ‘single’ linkage method, the height at which a cluster is connected with other clusters can be interpreted as the shortest distance (i.e. highest similarity) between any pair of strains in the two strain clusters.

FIG. 2. FIG. 2 depicts an overview of eps clusters of L. lactis of the invention. ORFs are annotated according to their proven or predicted functions, based on BLAST analysis on NCBI web-page against refseq protein database using default parameters. GT, glycosyltransferase; IS, transposase; hypot, hypothetical protein.

### BRIEF DESCRIPTION OF THE SEQUENCE LISTING

DSM 33134

SEQ ID NO:91 sets out the sets out the Lactococcus lactis strain DSM 33134 eps gene cluster,

complete sequence;

SEQ ID NO:1 sets out the open reading frame (ORF) of the epsR gene of DSM 33134;

SEQ ID NO:2 sets out the amino acid sequence encoded by SEQ ID NO:1;

SEQ ID NO:3 sets out the ORF of the epsX gene of DSM 33134;

SEQ ID NO:4 sets out the amino acid sequence encoded by SEQ ID NO:3;

SEQ ID NO:5 sets out the ORF of the epsB gene of DSM 33134;

SEQ ID NO:6 sets out the amino acid sequence encoded by SEQ ID NO:5;

SEQ ID NO:7 sets out the ORF of the epsD gene of DSM 33134

SEQ ID NO:8 sets out the amino acid sequence encoded by SEQ ID NO:7;

SEQ ID NO:9 sets out the ORF coding a putative GT1 protein of DSM 33134;

SEQ ID NO:10 sets out the amino acid sequence encoded by SEQ ID NO:9;

SEQ ID NO:11 sets out the ORF of a putative wzy gene of DSM 33134;

SEQ ID NO:12 sets out the amino acid sequence encoded by SEQ ID NO:11;

SEQ ID NO:13 sets out the ORF coding a putative GT2 protein of DSM 33134;

SEQ ID NO:14 sets out the amino acid sequence encoded by SEQ ID NO:13;

SEQ ID NO:15 sets out the ORF coding a putative GT3 protein of DSM 33134;

SEQ ID NO:16 sets out the amino acid sequence encoded by SEQ ID NO:15;

SEQ ID NO:17 sets out the ORF of a putative wzx gene of DSM 33134;

SEQ ID NO:18 sets out the amino acid sequence encoded by SEQ ID NO:17;

SEQ ID NO: 19 sets out the ORF of the epsL gene of DSM 33134;

SEQ ID NO:20 sets out the amino acid sequence encoded by SEQ ID NO:19;

SEQ ID NO:21 sets out the ORF coding a putative LytR family transcriptional regulator protein of DSM 33134;

SEQ ID NO:22 sets out the amino acid sequence encoded by SEQ ID NO:21;

SEQ ID NO:23 sets out the ORF coding a putative nucleotide sugar dehydrogenase protein of DSM 33134;

SEQ ID NO:24 sets out the amino acid sequence encoded by SEQ ID NO:23;

SEQ ID NO:25 sets out the ORF of the epsC gene of DSM 33134;

SEQ ID NO:26 sets out the amino acid sequence encoded by SEQ ID NO:25;

SEQ ID NO:27 sets out the ORF of the epsE gene of DSM 33134;

SEQ ID NO:28 sets out the amino acid sequence encoded by SEQ ID NO:27;

DSM 33136

SEQ ID NO:92 sets out the Lactococcus lactic strain DSM 33136 eps gene cluster, complete sequence;

SEQ ID NO:29 sets out the open reading frame (ORF) of the epsR gene of DSM 33136;

SEQ ID NO:30 sets out the amino acid sequence encoded by SEQ ID NO:29;

SEQ ID NO:31 sets out the ORF of the epsX gene of DSM 33136;

SEQ ID NO:32 sets out the amino acid sequence encoded by SEQ ID NO:31;

SEQ ID NO:33 sets out the ORF of the epsB gene of DSM 33136;

SEQ ID NO:34 sets out the amino acid sequence encoded by SEQ ID NO:33;

SEQ ID NO:35 sets out the ORF of the epsD gene of DSM 33136;

SEQ ID NO:36 sets out the amino acid sequence encoded by SEQ ID NO:35;

SEQ ID NO:37 sets out the ORF coding a putative GT1 protein of DSM 33136;

SEQ ID NO:38 sets out the amino acid sequence encoded by SEQ ID NO:37;

SEQ ID NO:39 sets out the ORF of a putative wzy gene of DSM 33136;

SEQ ID NO:40 sets out the amino acid sequence encoded by SEQ ID NO:39;

SEQ ID NO:41 sets out the ORF coding a putative GT2 protein of DSM 33136;

SEQ ID NO:42 sets out the amino acid sequence encoded by SEQ ID NO:41;

SEQ ID NO:43 sets out the ORF coding a putative GT3 protein of DSM 33136;

SEQ ID NO:44 sets out the amino acid sequence encoded by SEQ ID NO:43;

SEQ ID NO:45 sets out the ORF of a putative wzx gene of DSM 33136;

SEQ ID NO:46 sets out the amino acid sequence encoded by SEQ ID NO:45;

SEQ ID NO:47 sets out the ORF of the epsL gene of DSM 33136;

SEQ ID NO:48 sets out the amino acid sequence encoded by SEQ ID NO:47;

SEQ ID NO:49 sets out the ORF coding a putative LytR family transcriptional regulator protein of DSM 33136;

SEQ ID NO:50 sets out the amino acid sequence encoded by SEQ ID NO:49;

SEQ ID NO:51 sets out the ORF coding a putative polysaccharide pyruvyl transferase family protein of DSM 33136;

SEQ ID NO:52 sets out the amino acid sequence encoded by SEQ ID NO:51;

SEQ ID NO:53 sets out the ORF of the epsC gene of DSM 33136;

SEQ ID NO:54 sets out the amino acid sequence encoded by SEQ ID NO:53;

SEQ ID NO:55 sets out the ORF of the epsE gene of DSM 33136;

SEQ ID NO:56 sets out the amino acid sequence encoded by SEQ ID NO:55;

DSM 33139

SEQ ID NO:221 sets out the Lactococcus lactic strain DSM 33139 eps gene cluster, complete sequence;

SEQ ID NO:57 sets out the open reading frame (ORF) of the epsR gene of DSM 33139;

SEQ ID NO:58 sets out the amino acid sequence encoded by SEQ ID NO:57;

SEQ ID NO:59 sets out the ORF of the epsX gene of DSM 33139;

SEQ ID NO:60 sets out the amino acid sequence encoded by SEQ ID NO:59;

SEQ ID NO:61 sets out the ORF of the epsB gene of DSM 33139;

SEQ ID NO:62 sets out the amino acid sequence encoded by SEQ ID NO:61;

SEQ ID NO:63 sets out the ORF of the epsD gene of DSM 33139;

SEQ ID NO:64 sets out the amino acid sequence encoded by SEQ ID NO:63;

SEQ ID NO:65 sets out the ORF coding a putative GT1 protein of DSM 33139;

SEQ ID NO:66 sets out the amino acid sequence encoded by SEQ ID NO:65;

SEQ ID NO:67 sets out the ORF of a putative wzy gene of DSM 33139;

SEQ ID NO:68 sets out the amino acid sequence encoded by SEQ ID NO:67;

SEQ ID NO:69 sets out the ORF coding a putative GT2 protein of DSM 33139;

SEQ ID NO:70 sets out the amino acid sequence encoded by SEQ ID NO:69;

SEQ ID NO:71 sets out the ORF coding a putative GT3 protein of DSM 33139;

SEQ ID NO:72 sets out the amino acid sequence encoded by SEQ ID NO:71;

SEQ ID NO:73 sets out the ORF of a putative wzx gene of DSM 33139;

SEQ ID NO:74 sets out the amino acid sequence encoded by SEQ ID NO:73;

SEQ ID NO:75 sets out the ORF of the epsL gene of DSM 33139;

SEQ ID NO:76 sets out the amino acid sequence encoded by SEQ ID NO:75;

SEQ ID NO:77 sets out the ORF coding a putative LytR family transcriptional regulator protein of DSM 33139;

SEQ ID NO:78 sets out the amino acid sequence encoded by SEQ ID NO:77;

SEQ ID NO:79 sets out the ORF coding a putative nucleotide sugar dehydrogenase protein of DSM 33139;

SEQ ID NO:80 sets out the amino acid sequence encoded by SEQ ID NO:79;

SEQ ID NO:81 sets out the ORF of the epsC gene of DSM 33139;

SEQ ID NO:82 sets out the amino acid sequence encoded by SEQ ID NO:81;

SEQ ID NO:83 sets out the ORF of the epsE gene of DSM 33139;

SEQ ID NO:84 sets out the amino acid sequence encoded by SEQ ID NO:83;

SEQ ID NO:85 sets out the ORF coding a putative GT4 protein of DSM 33139;

SEQ ID NO:86 sets out the amino acid sequence encoded by SEQ ID NO:85;

SEQ ID NO:87 sets out the ORF coding a putative GT5 protein of DSM 33139;

SEQ ID NO:88 sets out the amino acid sequence encoded by SEQ ID NO:87;

SEQ ID NO:89 sets out the ORF coding a putative GT6 protein of DSM 33139;

SEQ ID NO:90 sets out the amino acid sequence encoded by SEQ ID NO:89;

SEQ ID NO:93 sets out the ORF coding a putative NAD-dependent epimerase/dehydratase family protein 1 of DSM 33139;

SEQ ID NO:94 sets out the amino acid sequence encoded by SEQ ID NO:93;

SEQ ID NO:95 sets out the ORF coding a putative glucose-1-phosphate thymidylyltransferase RfbA protein of DSM 33139;

SEQ ID NO:96 sets out the amino acid sequence encoded by SEQ ID NO:95;

SEQ ID NO:97 sets out the ORF coding a putative dTDP-glucose 4,6-dehydratase protein of DSM 33139;

SEQ ID NO:98 sets out the amino acid sequence encoded by SEQ ID NO:97;

SEQ ID NO:99 sets out the ORF coding a putative dTDP-4-dehydrorhamnose 3,5-epimerase protein of DSM 33139;

SEQ ID NO:100 sets out the amino acid sequence encoded by SEQ ID NO:99;

SEQ ID NO:101 sets out the ORF coding a putative NAD-dependent epimerase/dehydratase family protein 2 of DSM 33139;

SEQ ID NO:102 sets out the amino acid sequence encoded by SEQ ID NO:101;

SEQ ID NO:103 sets out the ORF coding a putative dTDP-4-dehydrorhamnose reductase protein of DSM 33139;

SEQ ID NO:104 sets out the amino acid sequence encoded by SEQ ID NO:103;

SEQ ID NO:105 sets out the ORF coding a putative nucleotidyl transferase protein of DSM 33139;

SEQ ID NO:106 sets out the amino acid sequence encoded by SEQ ID NO:105;

SEQ ID NO:107 sets out the ORF coding a putative acyltransferase 2 protein of DSM 33139;

SEQ ID NO:108 sets out the amino acid sequence encoded by SEQ ID NO:107;

SEQ ID NO:109 sets out the ORF coding a putative DUF1972 domain-containing protein of DSM 33139;

SEQ ID NO:110 sets out the amino acid sequence encoded by SEQ ID NO:109;

SEQ ID NO:111 sets out the ORF coding a putative acyltransferase 1 protein of DSM 33139;

SEQ ID NO:112 sets out the amino acid sequence encoded by SEQ ID NO:111;

DSM 33141

SEQ ID NO:222 sets out the Lactococcus lactic strain DSM 33141 eps gene cluster, complete sequence;

SEQ ID NO:113 sets out the open reading frame (ORF) of the epsR gene of DSM 33141;

SEQ ID NO:114 sets out the amino acid sequence encoded by SEQ ID NO:113;

SEQ ID NO:115 sets out the ORF of the epsX gene of DSM 33141;

SEQ ID NO:116 sets out the amino acid sequence encoded by SEQ ID NO:115;

SEQ ID NO:117 sets out the ORF of the epsB gene of DSM 33141;

SEQ ID NO:118 sets out the amino acid sequence encoded by SEQ ID NO:117;

SEQ ID NO:119 sets out the ORF of the epsD gene of DSM 33141

SEQ ID NO:120 sets out the amino acid sequence encoded by SEQ ID NO:119;

SEQ ID NO:121 sets out the ORF coding a putative GT1 protein of DSM 33141;

SEQ ID NO:122 sets out the amino acid sequence encoded by SEQ ID NO:121;

SEQ ID NO:123 sets out the ORF of a putative wzy gene of DSM 33141;

SEQ ID NO:124 sets out the amino acid sequence encoded by SEQ ID NO:123;

SEQ ID NO:125 sets out the ORF coding a putative GT2 protein of DSM 33141;

SEQ ID NO:126 sets out the amino acid sequence encoded by SEQ ID NO:125;

SEQ ID NO:127 sets out the ORF coding a putative GT3 protein of DSM 33141;

SEQ ID NO:128 sets out the amino acid sequence encoded by SEQ ID NO:127;

SEQ ID NO:129 sets out the ORF of a putative wzx gene of DSM 33141;

SEQ ID NO:130 sets out the amino acid sequence encoded by SEQ ID NO:129;

SEQ ID NO: 131 sets out the ORF of the epsL gene of DSM 33141;

SEQ ID NO:132 sets out the amino acid sequence encoded by SEQ ID NO:131;

SEQ ID NO:133 sets out the ORF coding a putative LytR family transcriptional regulator protein of DSM 33141;

SEQ ID NO:134 sets out the amino acid sequence encoded by SEQ ID NO:133;

SEQ ID NO:135 sets out the ORF coding a putative nucleotide sugar dehydrogenase protein of DSM 33141;

SEQ ID NO:136 sets out the amino acid sequence encoded by SEQ ID NO:135;

SEQ ID NO:137 sets out the ORF of the epsC gene of DSM 33141;

SEQ ID NO:138 sets out the amino acid sequence encoded by SEQ ID NO:137;

SEQ ID NO:139 sets out the ORF of the epsE1 gene of DSM 33141;

SEQ ID NO:140 sets out the amino acid sequence encoded by SEQ ID NO:139;

SEQ ID NO:141 sets out the ORF of the epsE2 gene of DSM 33141;

SEQ ID NO:142 sets out the amino acid sequence encoded by SEQ ID NO:141;

SEQ ID NO:143 sets out the ORF coding a putative GT4 protein of DSM 33141;

SEQ ID NO:144 sets out the amino acid sequence encoded by SEQ ID NO:143;

SEQ ID NO:145 sets out the ORF coding a putative GT5 protein of DSM 33141;

SEQ ID NO:146 sets out the amino acid sequence encoded by SEQ ID NO:145;

SEQ ID NO:147 sets out the ORF coding a putative GT6 protein of DSM 33141;

SEQ ID NO:148 sets out the amino acid sequence encoded by SEQ ID NO:147;

SEQ ID NO:149 sets out the ORF coding a putative acetyltransferase protein of DSM 33141;

SEQ ID NO:150 sets out the amino acid sequence encoded by SEQ ID NO:149;

SEQ ID NO:151 sets out the ORF coding a putative acyltransferase protein of DSM 33141;

SEQ ID NO:152 sets out the amino acid sequence encoded by SEQ ID NO:151;

DSM 33137

SEQ ID NO:223 sets out the Lactococcus lactic strain DSM 33137 eps gene cluster, complete sequence;

SEQ ID NO:153 sets out the open reading frame (ORF) of the epsR gene of DSM 33137;

SEQ ID NO:154 sets out the amino acid sequence encoded by SEQ ID NO:153;

SEQ ID NO:155 sets out the ORF of the epsX gene of DSM 33137;

SEQ ID NO:156 sets out the amino acid sequence encoded by SEQ ID NO:155;

SEQ ID NO:157 sets out the ORF of the epsB gene of DSM 33137;

SEQ ID NO:158 sets out the amino acid sequence encoded by SEQ ID NO:157;

SEQ ID NO:159 sets out the ORF of the epsD gene of DSM 33137;

SEQ ID NO:160 sets out the amino acid sequence encoded by SEQ ID NO:159;

SEQ ID NO:161 sets out the ORF coding a putative GT1 protein of DSM 33137;

SEQ ID NO:162 sets out the amino acid sequence encoded by SEQ ID NO:161;

SEQ ID NO:163 sets out the ORF of a putative wzy gene of DSM 33137;

SEQ ID NO:164 sets out the amino acid sequence encoded by SEQ ID NO:163;

SEQ ID NO:165 sets out the ORF coding a putative GT2 protein of DSM 33137;

SEQ ID NO:166 sets out the amino acid sequence encoded by SEQ ID NO:165;

SEQ ID NO:167 sets out the ORF coding a putative GT3 protein of DSM 33137;

SEQ ID NO:168 sets out the amino acid sequence encoded by SEQ ID NO:167;

SEQ ID NO:169 sets out the ORF of a putative wzx gene of DSM 33137;

SEQ ID NO:170 sets out the amino acid sequence encoded by SEQ ID NO:169;

SEQ ID NO:171 sets out the ORF of the epsL gene of DSM 33137;

SEQ ID NO:172 sets out the amino acid sequence encoded by SEQ ID NO:171;

SEQ ID NO:173 sets out the ORF coding a putative LytR family transcriptional regulator protein of DSM 33137;

SEQ ID NO:174 sets out the amino acid sequence encoded by SEQ ID NO:173;

SEQ ID NO:175 sets out the ORF coding a putative Core-2/I-Branching protein of DSM 33137;

SEQ ID NO:176 sets out the amino acid sequence encoded by SEQ ID NO:175;

SEQ ID NO:177 sets out the ORF of the epsC gene of DSM 33137;

SEQ ID NO:178 sets out the amino acid sequence encoded by SEQ ID NO:177;

SEQ ID NO:179 sets out the ORF of the epsE gene of DSM 33137;

SEQ ID NO:180 sets out the amino acid sequence encoded by SEQ ID NO:179;

SEQ ID NO:181 sets out the ORF coding a putative GT4 protein of DSM 33137;

SEQ ID NO:182 sets out the amino acid sequence encoded by SEQ ID NO:181;

DSM 33192

SEQ ID NO:183 sets out the Lactococcus lactic strain DSM 33192 eps gene cluster, complete sequence;

SEQ ID NO:184 sets out the amino acid sequence encoded by the epsR gene of DSM 33192 (nucleotides 1-318 of SEQ ID NO:183);

SEQ ID NO:185 sets out the amino acid sequence encoded by the epsX gene of DSM 33192 (nucleotides 407-826 of SEQ ID NO:183);

SEQ ID NO:186 sets out the amino acid sequence encoded by the epsC gene of DSM 33192 (nucleotides 993-1772 of SEQ ID NO:183);

SEQ ID NO:187 sets out the amino acid sequence encoded by the epsD gene of DSM 33192 (nucleotides 1782-2477 of SEQ ID NO:183);

SEQ ID NO:188 sets out the amino acid sequence encoded by the epsB gene of DSM 33192 (nucleotides 2532-3296 of SEQ ID NO:183);

SEQ ID NO:189 sets out the amino acid sequence encoded by the epsE gene of DSM 33192 (nucleotides 3318-3998 of SEQ ID NO:183);

SEQ ID NO:190 sets out the amino acid sequence of a putative glycosyltransferase (GT1) of DSM 33192, encoded by nucleotides 4008-4478 of SEQ ID NO:183;

SEQ ID NO:191 sets out the amino acid sequence of a putative glycosyltransferase (GT2) of DSM 33192, encoded by nucleotides 4478-4960 of SEQ ID NO:183;

SEQ ID NO:192 sets out the amino acid sequence of a putative glycosyltransferase (GT3) of DSM 33192, encoded by nucleotides 5015-5965 of SEQ ID NO:183;

SEQ ID NO:193 sets out the amino acid sequence of a putative glycosyltransferase (GT4) of DSM 33192, encoded by nucleotides 6026-6955 of SEQ ID NO:183;

SEQ ID NO:194 sets out the amino acid sequence encoded by the wzy gene of DSM 33192 (nucleotides 6955-8145 of SEQ ID NO:183);

SEQ ID NO:195 sets out the amino acid sequence of a glycerophosphotransferase family protein of DSM 33192, encoded by nucleotides 8132-9322 of SEQ ID NO:183;

SEQ ID NO:196 sets out the amino acid sequence encoded by the wzx gene of DSM 33192 (nucleotides 9309-10727 of SEQ ID NO:183);

SEQ ID NO:197 sets out the amino acid sequence encoded by the epsL gene of DSM 33192 (nucleotides 10825-11724 of SEQ ID NO:183);

SEQ ID NO:198 sets out the amino acid sequence of LytR protein of DSM 33192, encoded by nucleotides 11749-12651 of SEQ ID NO:183;

DSM 33135

SEQ ID NO:199 sets out the Lactococcus lactic strain DSM 33135 eps gene cluster, complete sequence;

SEQ ID NO:200 sets out the amino acid sequence encoded by the epsR gene of DSM 33135 (nucleotides 1-318 of SEQ ID NO:199);

SEQ ID NO:201 sets out the amino acid sequence encoded by the epsX gene of DSM 33135 (nucleotides 352-1119 of SEQ ID NO:199);

SEQ ID NO:202 sets out the amino acid sequence encoded by the epsC gene of DSM 33135 (nucleotides 1159 to 1938 of SEQ ID NO:199);

SEQ ID NO:203 sets out the amino acid sequence encoded by the epsD gene of DSM 33135 (nucleotides 1948-2640 of SEQ ID NO:199);

SEQ ID NO:204 sets out the amino acid sequence encoded by the epsB gene of DSM 33135 (nucleotides 2698 to 3462 of SEQ ID NO:199);

SEQ ID NO:205 sets out the amino acid sequence encoded by the epsE gene of DSM 33135 (nucleotides 3484-4170 of SEQ ID NO:199);

SEQ ID NO:206 sets out the amino acid sequence of a putative glycosyltransferase (GT1) of DSM 33135, encoded by nucleotides 4174-4824 of SEQ ID NO:199;

SEQ ID NO:207 sets out the amino acid sequence of a putative dTDP-glucose 4,6-dehydratase of DSM 33135, encoded by nucleotides 4784-5695 of SEQ ID NO:199;

SEQ ID NO:208 sets out the amino acid sequence of a putative dTDP-4-dehydrorhamnose reductase of DSM 33135, encoded by nucleotides 5717-6631 of SEQ ID NO:199;

SEQ ID NO:209 sets out the amino acid sequence of a putative dTDP-4-dehydrorhamnose 3,5-epimerase of DSM 33135, encoded by nucleotides 6586-7257 of SEQ ID NO:199;

SEQ ID NO:210 sets out the amino acid sequence of a putative glycosyltransferase (GT2) of DSM 33135 encoded by nucleotides 7276-8508 of SEQ ID NO:199;

SEQ ID NO:211 sets out the amino acid sequence of a putative DUF1919 protein of DSM 33135, encoded by nucleotides 8515-9144 of SEQ ID NO:199;

SEQ ID NO:212 sets out the amino acid sequence of a putative UDP-galactopyranose mutase protein of DSM 33135 encoded by nucleotides 9159-10274 of SEQ ID NO:199;

SEQ ID NO:213 sets out the amino acid sequence of a putative DUF4422 protein of DSM 33135 encoded by nucleotides 10271-11029 of SEQ ID NO:199;

SEQ ID NO:214 sets out the amino acid sequence of a putative glycosyltransferase (GT3) of DSM 33135, encoded by nucleotides 11042-12391 of SEQ ID NO:199;

SEQ ID NO:215 sets out the amino acid sequence of a putative glycosyltransferase (GT4) of DSM 33135, encoded by nucleotides 13008-13934 of SEQ ID NO:199;

SEQ ID NO:216 sets out the amino acid sequence encoded by the wzy gene of DSM 33135, (nucleotides 13939-15042 of SEQ ID NO:199);

SEQ ID NO:217 sets out the amino acid sequence of a putative glycosyltransferase (GT5) of DSM 33135, encoded by nucleotides 18528-19508 of SEQ ID NO:199;

SEQ ID NO:218 sets out the amino acid sequence of the lytR protein of DSM 33135, encoded by nucleotides 20389-21291 of SEQ ID NO:199;

SEQ ID NO:219 sets out the amino acid sequence encoded by the epsL gene of DSM 33135, (nucleotides 24053-24751 of the complementary strand of SEQ ID NO:199);

SEQ ID NO:220 sets out the amino acid sequence encoded by the wzx gene of DSM 33135, (nucleotides 26029-27444 of the complementary strand of SEQ ID NO:199);

DSM 33138

SEQ ID NO:224 sets out the Lactococcus lactic strain DSM 33138 eps gene cluster, complete sequence;

SEQ ID NO:225 sets out the amino acid sequence encoded by the epsR gene of DSM 33138 (nucleotides 1-318 of SEQ ID NO: 224);

SEQ ID NO:226 sets out the amino acid sequence encoded by the epsX gene of DSM 33138 (nucleotides 352-1119 of SEQ ID NO: 224);

SEQ ID NO:227 sets out the amino acid sequence encoded by the epsC gene of DSM 33138 (nucleotides 1159-1938 of SEQ ID NO: 224);

SEQ ID NO:228 sets out the amino acid sequence encoded by the epsD gene of DSM 33138 (nucleotides 1948-2640 of SEQ ID NO: 224);

SEQ ID NO:229 sets out the amino acid sequence encoded by the epsB gene of DSM 33138 (nucleotides 2698-3462 of SEQ ID NO: 224);

SEQ ID NO:230 sets out the amino acid sequence encoded by the epsE gene of DSM 33138 (nucleotides 3484-4170 of SEQ ID NO: 224);

SEQ ID NO:231 sets out the amino acid sequence of a putative glycosyltransferase (GT1) of DSM 33138, encoded by nucleotides 4174-4824 of SEQ ID NO: 224;

SEQ ID NO:232 sets out the amino acid sequence of a putative dTDP-glucose 4,6-dehydratase of DSM 33138, encoded by nucleotides 4784-5695 of SEQ ID NO: 224;

SEQ ID NO:233 sets out the amino acid sequence of a putative dTDP-4-dehydrorhamnose reductase of DSM 33138, encoded by nucleotides 5717-6631 of SEQ ID NO: 224;

SEQ ID NO:234 sets out the amino acid sequence of a putative dTDP-4-dehydrorhamnose 3,5-epimerase of DSM 33138, encoded by nucleotides 6586-7257 of SEQ ID NO: 224;

SEQ ID NO:235 sets out the amino acid sequence of a putative DUF1972 protein of DSM 33138, encoded by nucleotides 7276-8508 of SEQ ID NO: 224;

SEQ ID NO:236 sets out the amino acid sequence of a putative DUF1919 protein of DSM 33138, encoded by nucleotides 8515-9144 of SEQ ID NO: 224;

SEQ ID NO:237 sets out the amino acid sequence of a putative UDP-galactopyranose mutase protein of DSM 33138 encoded by nucleotides 9159-10274 of SEQ ID NO: 224;

SEQ ID NO:238 sets out the amino acid sequence of a putative DUF4422 protein of DSM 33138 encoded by nucleotides 10271-11029 of SEQ ID NO: 224;

SEQ ID NO:239 sets out the amino acid sequence of a putative glycosyltransferase (GT2) of DSM 33138, encoded by nucleotides 11042-12391 of SEQ ID NO: 224;

SEQ ID NO:240 sets out the amino acid sequence of a putative glycosyltransferase (GT3) of DSM 33138, encoded by nucleotides 13008-13934 of SEQ ID NO: 224;

SEQ ID NO:241 sets out the amino acid sequence encoded by the wzy gene of DSM 33138, (nucleotides 13939-15042 of SEQ ID NO: 224);

SEQ ID NO:242 sets out the amino acid sequence of a putative glycosyltransferase (GT4) of DSM 33138, encoded by nucleotides 18527-19507 of SEQ ID NO: 224;

SEQ ID NO:243 sets out the amino acid sequence of the lytR protein of DSM 33138, encoded by nucleotides 20388-21290 of SEQ ID NO: 224;

DSM 33140

SEQ ID NO:244 sets out the Lactococcus lactic strain DSM 33140 eps gene cluster, complete sequence;

SEQ ID NO:245 sets out the amino acid sequence encoded by the epsR gene of DSM 33140 (nucleotides 1-318 of SEQ ID NO: 244);

SEQ ID NO:246 sets out the amino acid sequence encoded by the epsX gene of DSM 33140 (nucleotides 352-1119 of SEQ ID NO: 244);

SEQ ID NO:247 sets out the amino acid sequence encoded by the epsC gene of DSM 33140 (nucleotides 1159-1938 of SEQ ID NO: 244);

SEQ ID NO:248 sets out the amino acid sequence encoded by the epsD gene of DSM 33140 (nucleotides 1948-2643 of SEQ ID NO: 244);

SEQ ID NO:249 sets out the amino acid sequence encoded by the epsB gene of DSM 33140 (nucleotides 2698-3462 of SEQ ID NO: 244);

SEQ ID NO:250 sets out the amino acid sequence encoded by the epsE gene of DSM 33140 (nucleotides 3484-4164 of SEQ ID NO: 244);

SEQ ID NO:251 sets out the amino acid sequence of a putative UDP-N-acetylglucosamine-LPS N-acetylglucosamine protein of DSM 33140, encoded by nucleotides 4168-4617 of SEQ ID NO: 244;

SEQ ID NO:252 sets out the amino acid sequence of a putative glycosyltransferase (GT1) of DSM 33140, encoded by nucleotides 4617-5123 of SEQ ID NO: 244;

SEQ ID NO:253 sets out the amino acid sequence of a putative glycosyltransferase (GT2) of DSM 33140, encoded by nucleotides 5120-5827 of SEQ ID NO: 244;

SEQ ID NO:254 sets out the amino acid sequence encoded by the gene wzy of DSM 33140 (nucleotides 5833-6927 of SEQ ID NO: 244);

SEQ ID NO:255 sets out the amino acid sequence encoded by the epsL gene of DSM 33140, (nucleotides 8438-9340 of SEQ ID NO: 244);

SEQ ID NO:256 sets out the amino acid sequence of the lytR protein of DSM 33140, encoded by nucleotides 9365-10267 of the complementary strand of SEQ ID NO: 244;

DSM 33142

SEQ ID NO:257 sets out the Lactococcus lactic strain DSM 33142 eps gene cluster, complete sequence;

SEQ ID NO:258 sets out the amino acid sequence encoded by the epsR gene of DSM 33142 (nucleotides 1-318 of SEQ ID NO: 257);

SEQ ID NO:259 sets out the amino acid sequence encoded by the epsX gene of DSM 33142 (nucleotides 407-1120 of SEQ ID NO: 257);

SEQ ID NO:260 sets out the amino acid sequence encoded by the epsC gene of DSM 33142 (nucleotides 1160-1939 of SEQ ID NO: 257);

SEQ ID NO:261 sets out the amino acid sequence encoded by the epsD gene of DSM 33142 (nucleotides 1949-2644 of SEQ ID NO: 257);

SEQ ID NO:262 sets out the amino acid sequence encoded by the epsB gene of DSM 33142 (nucleotides 2699-3463 of SEQ ID NO: 257);

SEQ ID NO:263 sets out the amino acid sequence encoded by the epsE1 gene of DSM 33142 (nucleotides 3485-4084 of SEQ ID NO: 257);

SEQ ID NO:264 sets out the amino acid sequence encoded by the epsE2 gene of DSM 33142 (nucleotides 4085-4840 of SEQ ID NO: 257);

SEQ ID NO:265 sets out the amino acid sequence of lytR protein of DSM 33142, encoded by nucleotides 5876-6778 of SEQ ID NO: 257;

SEQ ID NO:266 sets out the amino acid sequence encoded by the epsL gene of DSM 33142, encoded by nucleotides 6803-7717 of the complementary strand of SEQ ID NO: 257;

SEQ ID NO:267 sets out the amino acid sequence of a putative nucleotide sugar dehydrogenase protein of DSM 33142, encoded by nucleotides 7727-8173 of the complementary strand of SEQ ID NO: 257;

SEQ ID NO:268 sets out the amino acid sequence of a putative glycosyltransferase (GT1) of DSM 33142, encoded by nucleotides 9726-10673 of the complementary strand of SEQ ID NO: 257;

SEQ ID NO:269 sets out the amino acid sequence of a putative acetyltransferase of DSM 33142, encoded by nucleotides 10657-11211 of the complementary strand of SEQ ID NO: 257;

SEQ ID NO:270 sets out the amino acid sequence encoded by the wzy gene of DSM 33142, (nucleotides 11201-12349 of the complementary strand of SEQ ID NO: 257);

SEQ ID NO:271 sets out the amino acid sequence of a putative glycosyltransferase (GT2) of DSM 33142, encoded by nucleotides 12336-13421 of the complementary strand of SEQ ID NO: 257;

SEQ ID NO:272 sets out the amino acid sequence of a putative glycosyltransferase (GT3) of DSM 33142, encoded by nucleotides 13418-14260 of the complementary strand of SEQ ID NO: 257;

SEQ ID NO:273 sets out the amino acid sequence encoded by the wzx gene of the complementary strand of DSM 33142, (nucleotides 15538-16953 of SEQ ID NO: 257);

DSM 33183

SEQ ID NO:274 sets out the Lactococcus lactic strain DSM 33183 eps gene cluster, complete sequence;

SEQ ID NO:275 sets out the amino acid sequence encoded by the epsR gene of DSM 33183 (nucleotides 1-318 of SEQ ID NO: 274);

SEQ ID NO:276 sets out the amino acid sequence encoded by the epsC gene of DSM 33183 (nucleotides 681-1460 of SEQ ID NO: 274);

SEQ ID NO:277 sets out the amino acid sequence encoded by the epsD gene of DSM 33183 (nucleotides 1470-2165 of SEQ ID NO: 274);

SEQ ID NO:278 sets out the amino acid sequence encoded by the epsB gene of DSM 33183 (nucleotides 2220-3005 of SEQ ID NO: 274);

SEQ ID NO:279 sets out the amino acid sequence encoded by the epsE1 gene of DSM 33183 (nucleotides 2992-3591 of SEQ ID NO: 274);

SEQ ID NO:280 sets out the amino acid sequence encoded by the epsE2 gene of DSM 33183 (nucleotides 3592-4347 of SEQ ID NO: 274);

SEQ ID NO:281 sets out the amino acid sequence of lytR protein of DSM 33183, encoded by nucleotides 5383-6285 of SEQ ID NO: 274);

SEQ ID NO:282 sets out the amino acid sequence encoded by the epsL gene of DSM 33183, encoded by nucleotides 6310-7224 of the complementary strand of SEQ ID NO: 274);

SEQ ID NO:283 sets out the amino acid sequence of a putative nucleotide sugar dehydrogenase protein of DSM 33183, encoded by nucleotides 7234-7680 of the complementary strand of SEQ ID NO: 274;

SEQ ID NO:284 sets out the amino acid sequence of a putative glycosyltransferase (GT1) of DSM 33183 encoded by nucleotides 9232-10179 of the complementary strand of SEQ ID NO: 274;

SEQ ID NO:285 sets out the amino acid sequence of a putative acetyltransferase of DSM 33183, encoded by nucleotides 10163-10717 of the complementary strand of SEQ ID NO: 274;

SEQ ID NO:286 sets out the amino acid sequence encoded by the wzy gene of DSM 33183, (nucleotides 10707-11846 of the complementary strand of SEQ ID NO: 274);

SEQ ID NO:287 sets out the amino acid sequence of a putative glycosyltransferase (GT2) of DSM 33183, encoded by nucleotides 11833-12918 of the complementary strand of SEQ ID NO: 274;

SEQ ID NO:288 sets out the amino acid sequence of a putative glycosyltransferase (GT3) of DSM 33183, encoded by nucleotides 12915-13757 of the complementary strand of SEQ ID NO: 274;

SEQ ID NO:289 sets out the amino acid sequence encoded by the wzx gene of DSM 33183, (nucleotides 15037-16476 of the complementary strand of SEQ ID NO: 274).

SEQ ID NO:290 sets out the Lactococcus lactis strain DSM 33133 eps gene cluster, complete sequence.

SEQ ID NO:291 sets out the Lactococcus lactis strains DSM 33204, 33205, 33220, 33221, 33218, 33219, 33224, 33197, 33196, 33195, 33194, 33226, 33223, 33193 and 33192 eps gene clusters, complete sequence.

SEQ ID NO:292 sets out the Lactococcus lactis strains DSM 33200, 33201, 33202 and 33203 eps gene clusters, complete sequence.

SEQ ID NO:293 sets out the Lactococcus lactis strain DSM 33222 eps gene cluster, complete sequence.

SEQ ID NO:294 sets out the Lactococcus lactis strain DSM 33225 eps gene cluster, complete sequence.

### DETAILED DESCRIPTION

### Definitions

All definitions of herein relevant terms are in accordance of what would be understood by the skilled person in relation to the herein relevant technical context.

In the context of the present invention in any of its embodiments, the expression “lactic acid bacteria” (“LAB”) designates food-grade bacteria producing lactic acid as the major metabolic end-product of carbohydrate fermentation. These bacteria are related by their common metabolic and physiological characteristics and are usually Gram positive, low-GC, acid tolerant, non-sporulating, non-respiring, rod-shaped bacilli or cocci. During the fermentation stage, the consumption of carbohydrate by these bacteria causes the formation of lactic acid, reducing the pH and leading to the formation of a protein coagulum. These bacteria are thus responsible for the acidification of milk and for the texture of the dairy product. The industrially most useful lactic acid bacteria are found within the order “Lactobacillales” which includes Lactococcus spp., Streptococcus spp., Lactobacillus spp., Leuconostoc spp., Pediococcus spp. and Propionibacterium spp. These are frequently used as food cultures alone or in combination with other lactic acid bacteria.

By “texturing strain” in the present specification and claims is meant a strain which preferably generates fermented mammalian milks having, under the conditions described below and as exemplified in Example 1 herein, a shear stress preferably greater than 40 Pa measured at shear rate 300 s⁻¹. A strain of Lactococcus lactic can be defined as strongly texturing in that it generates fermented milks having, under the same conditions, a shear stress greater than 50 Pa measured at shear rate 300 s⁻¹. 200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 followed by storage at 4° C. until shear stress is measured, typically from 1-7 days, such as for 5 days, followed by gently stirring and measuring the shear stress at shear rate 300 s⁻¹ wherein the inoculation temperature is 30° C.

In addition, by “texturing strain” in the present specification and claims is meant a strain which preferably generates fermented plant-based milks having, under the conditions described below and as exemplified in Example 2 herein, a shear stress of 24 Pa or more, preferably 30 Pa or more, or even more preferably 42 Pa or more than measured at shear rate 300 s⁻¹. A strain of Lactococcus lactic can be defined as strongly texturing in that it generates fermented milks having, under the same conditions, a shear stress of 30 Pa or more measured at shear rate 300 s⁻¹. 1% volume overnight microbial culture (obtained by inoculating the microbial culture in M17 broth supplemented with 2% glucose at 30° C.) is inoculated in soy milk with glucose, such as 0.5-5% glucose, preferably 0.5-2% glucose, more preferably about 2% glucose, as described in Example 2. The inoculation takes place at 30° C. in 200-ml scale until target pH has been reached e.g. pH between 4 and 5, preferably between pH 4.3 to 4.7, more preferably between pH 4.4 to 4.6, and even more preferably pH 4.45, pH 4.50, or pH 4.55 followed by cooling to 4° C. and storage at 4° C. until shear stress is measured, typically from 1-7 days, such as for 5 days, followed by gently stirring and measuring the shear stress at shear rate 300 s⁻¹ wherein the inoculation temperature is 30° C.

The texturing lactic acid bacterium strain of the invention may be an isolated strain, e.g., isolated from a naturally occurring source, or may be a non-naturally occurring strain, e.g., obtained recombinantly. Recombinant strains will differ from naturally occurring strains by at least the presence of the nucleic acid construct(s) used to transform or transfect the mother strain.

The term “sequence identity” relates to the relatedness between two nucleotide sequences or between two amino acid sequences. For purposes of the present invention, the degree of sequence identity between two nucleotide sequences or two amino acid sequences is determined using multiple sequence alignment tool Clustal Omega (www.ebi.ac.uk/Tools/msa/clustalo/; Sievers, F. et al., 2011, “Fast, scalable generation of high-quality protein multiple sequence alignments using Clustal Omega”, Mol. Syst. Biol., 7:539) with standard parameters.

In the present context, the terms “strains derived from”, “derived strain” or “mutant” should be understood as a strain derived from a strain of the invention by means of, e.g., genetic engineering, radiation and/or chemical treatment, and/or selection, adaptation, screening, etc. It is preferred that the derived strain is a functionally equivalent mutant, e.g., a strain that has substantially the same, or improved, properties with respect to texturing capacity as the mother strain. Such a derived strain is a part of the present invention. Especially, the term “derived strain” or “mutant” refers to a strain obtained by subjecting a strain of the invention to any conventionally used mutagenesis treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenesis treatments (a single treatment should be understood one mutagenesis step followed by a screening/selection step), but it is presently preferred that no more than 20, no more than 10, or no more than 5, treatments are carried out. In a presently preferred derived strain, less than 1%, or less than 0.1%, less than 0.01%, less than 0.001% or even less than 0.0001% of the nucleotides in the bacterial genome have been changed (such as by replacement, insertion, deletion or a combination thereof) compared to the mother strain.

The term “thermophilic” herein refers to microorganisms that thrive best at temperatures above 35° C. The industrially most useful thermophilic bacteria include Streptococcus spp. and Lactobacillus spp. The term “thermophilic fermentation” herein refers to fermentation at a temperature above about 35° C., such as between about 35° C. to about 45° C. The term “thermophilic fermented milk product” refers to fermented milk products prepared by thermophilic fermentation of a thermophilic starter culture and include such fermented milk products as set-yoghurt, stirred-yoghurt and drinking yoghurt, e.g., Yakult. In addition, the term “thermophilic fermented milk product” refers to fermented milk products prepared by thermophilic fermentation of a thermophilic starter culture in a plant-based milk base, such as soy milk or soy milk supplemented with sugar such as e.g. fructose, sucrose, High Fructose Corn Syrup (HFCS), honey, glucose, invert sugar, maltose, galactose, lactose, or any combination thereof. The concentration of sugar may be between 0.5% to 5%, from 0.5 to 2%, 0.5%, 1%, 1.5%, or 2% such as e.g. 0.5-5% glucose, preferably 0.5-2% glucose, more preferably about 2% glucose.

The term “mesophilic” herein refers to microorganisms that thrive best at moderate temperatures (15° C.-35° C.). The industrially most useful mesophilic bacteria include Lactococcus spp. and Leuconostoc spp. The term “mesophilic fermentation” herein refers to fermentation at a temperature between about 22° C. and about 35° C. The term “mesophilic food products” refers to food products prepared by mesophilic fermentation of a mesophilic starter culture. The term “mesophilic fermented milk product” refers to fermented milk products prepared by mesophilic fermentation of a mesophilic starter culture and include such fermented milk products as buttermilk, sour milk, cultured milk, smetana, sour cream, Kefir and fresh cheese, such as quark, tvarog and cream cheese. In addition, the term “mesophilic fermented milk product” refers to fermented milk products prepared by mesophilic fermentation of a mesophilic starter culture in a plant-based milk base, such as soy milk or soy milk supplemented with sugar such as e.g. fructose, sucrose, High Fructose Corn Syrup (HFCS), honey, glucose, invert sugar, maltose, galactose, lactose, or any combination thereof. The concentration of sugar may be between 0.5% to 5%, from 0.5 to 2%, 0.5%, 1%, 1.5%, or 2% such as e.g. 0.5-5% glucose, preferably 0.5-2% glucose, more preferably about 2% glucose.

The term “mesophilic starter culture” herein refers to any starter cultures culture containing at least one mesophilic bacterium strain. Mesophilic starter cultures, such as combinations of Lactococcus lactis subsp lactis strains and Lactococcus lactis subsp. cremoris strains, are used to produce fermented milk products, such as fresh cheese, butter milk, sour milk and sour cream.

The terms “fermented milk” and “dairy” are used interchangeably herein. In the context of the present invention in any of its embodiments, the expression “fermented milk product” means a food or feed product wherein the preparation of the food or feed product involves fermentation of a milk base with a lactic acid bacterium. “Fermented milk product” as used herein includes but is not limited to products such as thermophilic fermented milk products or mesophilic fermented milk products, as defined above. In addition, as described above, “fermented milk product” as used herein includes products prepared by fermentation of plant-based milk bases, such as soy milk or soy milk supplemented with sugar such as e.g. fructose, sucrose, High Fructose Corn Syrup (HFCS), honey, glucose, invert sugar, maltose, galactose, lactose, or any combination thereof. The concentration of sugar may be between 0.5% to 5%, from 0.5 to 2%, 0.5%, 1%, 1.5%, or 2% such as e.g. 0.5-5% glucose, preferably 0.5-2% glucose, more preferably about 2% glucose. Hence, “fermented milk product” according to the present invention encompass fermented mammalian milk products (i.e., the milk base has mammalian origin) and fermented plant-milk product (i.e., the milk base is a plant-derived milk base, such as soy milk base).

In the context of the present application, the term “milk” is broadly used in its common meaning to refer to liquids produced by the mammary glands of animals (e.g., cows, sheep, goats, buffaloes, camel, etc.) or derived from plants. The term “milk base” or “milk substrate” may be any milk material that can be subjected to fermentation according to the present invention. Thus, useful milk bases include, but are not limited to, solutions/-suspensions of any milk or milk like products comprising protein, such as whole or low-fat milk, skim milk, buttermilk, reconstituted milk powder, condensed milk, dried milk, whey, whey permeate, lactose, mother liquid from crystallization of lactose, whey protein concentrate, cream or plant-based milks. Obviously, the milk base may originate from any mammalian, e.g., being substantially pure mammalian milk, or reconstituted milk powder. Plant sources of milk include, but are not limited to, milk extracted from soy bean. Preferably, the plant-based milk is soy milk, which can be preferably supplemented with sugar such as e.g. fructose, sucrose, High Fructose Corn Syrup (HFCS), honey, glucose, invert sugar, maltose, galactose, lactose, or any combination thereof. The concentration of sugar may be between 0.5% to 5%, from 0.5 to 2%, 0.5%, 1%, 1.5%, or 2% such as e.g. 0.5-5% glucose, preferably 0.5-2% glucose, more preferably about 2% glucose.

Prior to fermentation, the milk base may be homogenized and pasteurized according to methods known in the art. “Homogenizing” as used in the context of the present invention in any of its embodiments, means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurizing” as used in the context of the present invention in any of its embodiments, means treatment of the milk base to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow. For instance, milk base may be heat treated at 92° C. for 3 min, cooled to 38° C. and then inoculated as described in step i. of the process of the present invention.

As used herein, the term “about” (or “around”) means the indicated value±1% of its value, or the term “about” means the indicated value±2% of its value, or the term “about” means the indicated value±5% of its value, the term “about” means the indicated value±10% of its value, or the term “about” means the indicated value±20% of its value, or the term “about” means the indicated value±30% of its value; preferably the term “about” means exactly the indicated value (±0%).

Throughout the description and claims the word “comprise” and variations of the word (e.g., “comprising”, “having”, “including”, “containing”) typically is not limiting and thus does not exclude other features, which may be for example technical features, additives, components, or steps. However, whenever the word “comprise” is used herein, this also includes a special embodiment in which this word is understood as limiting; in this particular embodiment the word “comprise” has the meaning of the term “consist of”.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Texture is an important quality factor for fermented milk products such as yoghurt, and consumer acceptance is often closely linked to texture properties. The texture of fermented milk is dependent on both the bacteria used for fermentation and process parameters. Polysaccharide-producing bacteria can positively influence product characteristics such as texture and sensory properties. Sensory textural attributes are often correlated with the results from instrumental text, e.g., shear stress is related to viscosity and perceived mouth thickness (Poulsen et al., 2019). In the context of the present invention, the rheological properties (texture) of a fermented milk product, such as viscosity, can be measured as a function of the shear stress of the fermented milk product, as described below.

In connection with the present invention, shear stress may be measured by the following method: When the pH of the fermented milk (e.g., mammalian- or plant-based milk) reached pH^{˜}4.55, the fermented milk product was brought to 4° C. and manually stirred gently by means of a stick fitted with a perforated disc until homogeneity of the sample. The rheological properties of the sample were assessed on a rheometer (Anton Paar Physica Rheometer with ASC, Automatic Sample Changer, Anton Paar® GmbH, Austria) by using a bob-cup. The rheometer was set to a constant temperature of 13° C. during the time of measurement. Settings were as follows:
Holding time (to rebuild to somewhat original structure)
5 minutes without any physical stress (oscillation or rotation) applied to the sample.
Oscillation step (to measure the elastic and viscous modulus, G′ and G″, respectively, therefore calculating the complex modulus G*)
   Constant strain=0.3%, frequency (f)=[0.5 . . . 8] Hz
   6 measuring points over 60 s (one every 10 s)
Rotation step (to measure shear stress at 300 1/s)
Two steps were designed:
Shear rate=[0.3−300] 1/s and 2) Shear rate=[275−0.3] 1/s.

Each step contained 21 measuring points over 210 s (on every 10 s). The shear stress at 300 1/s (300 s⁻¹) was chosen for further analysis, as this correlates to mouth thickness when swallowing a fermented milk product.

Preferably, the shear stress may be measured by the following method: Shear stress data were obtained by inoculating the same microbial cultures in semi-fat milk (1.5% fat); milk was heated at 90° C. for 20 min and cooled down to the inoculation temperature (30° C.), prior to inoculation with 1% overnight microbial culture. The inoculation took place for 8-22 h at 30° C. in 200-ml scale until pH^{˜}4.55 followed by cooling to 4° C. and storage at 4° C. until shear stress was measured, typically from 1-7 days, such as for 5 days. After the storage, the fermented milk was stirred gently by means of a stick fitted with a bored disc until homogeneity of the sample. Shear stress of the samples was assessed on a rheometer (Anton Paar Physica Rheometer with ASC, Automatic Sample Changer, Anton Paar® GmbH, Austria) using the following settings:
Wait time (to rebuild to somewhat original structure)
5 minutes without oscillation or rotation
Rotation (to measure shear stress at 300 s⁻¹ etc.)
Y′=[0.2707−300] s⁻¹ and y′=[275−0.2707] s⁻¹

21 measuring points over 210 s (on every 10 s) going up to 300 s⁻¹ and 21 measuring points over 210 s (one every 10 s) going down to 0.2707 s⁻¹. For the data analysis, the shear stress at shear rate 300 s⁻¹ was chosen.

Alternatively, the shear stress is measured by the following method: 1% volume overnight microbial culture (obtained by inoculating the microbial culture in M17 broth supplemented with 2% glucose at 30° C.) is inoculated in soy milk with glucose, such as 0.5-5% glucose, preferably 0.5-2% glucose, more preferably about 2% glucose. The inoculation takes place at 30° C. in 200-ml scale until target pH, followed by cooling to 4° C. and storage at 4° C. until shear stress is measured, typically from 1-7 days, such as for 5 days. Target pH may be e.g. pH between 4 and 5, preferably between pH 4.3 to 4.7, more preferably between pH 4.4 to 4.6, and even more preferably pH 4.45, pH 4.50, or pH 4.55. After the storage, the fermented milk is stirred gently by means of a stick fitted with a bored disc until homogeneity of the sample. Shear stress is assessed on a rheometer (Anton Paar Physica Rheometer with ASC, Automatic Sample Changer, Anton Paar® GmbH, Austria) using the following settings:
Wait time (to rebuild to somewhat original structure)
5 minutes without oscillation or rotation
Rotation (to measure shear stress at 300 s⁻¹ etc.)
Y′=[0.2707−300] s⁻¹ and y′=[275−0.2707] s⁻¹

21 measuring points over 210 s (on every 10 s) going up to 300 s⁻¹ and 21 measuring points over 210 s (one every 10 s) going down to 0.2707 s⁻¹. For the data analysis, the shear stress at shear rate 300 s⁻¹ may be chosen.

The Lactococcus lactis Subsp. Lactis Lactic Acid Bacterium (LAB) Strains

It is an object of the present invention to provide texturing LAB strains suitable for use in preparation of food products. In particular, it is an object of the present invention to provide texturing Lactococcus lactis strains suitable for use in preparation of mesophilic food products. This object has been solved with the Lactococcus lactis strains comprising novel eps gene clusters, as described herein. As discussed in the examples (see, e.g., Tables 1 and 2 and Examples 1 and 2), the disclosed Lactococcus lactis strains DSM 33134, DSM 33135, DSM 33136, DSM 33137, DSM 33138, DSM 33139, DSM 33140, DSM 33141, DSM 33142, DSM 33183, DSM 25485 and DSM 33192 have excellent texturing properties.

The present inventors analyzed the eps gene cluster of the above strains and identified novel gene sequences which are believed to be involved in the production of exopolysaccharide (EPS), and thereby involved in the creation of the excellent texturing properties of the above Lactococcus lactis strains for fermenting milk.

Without being limited to theory, there is no substantial reason to believe that it would not be plausible that another Lactococcus lactis strain (i.e., different from the specific strains of the invention) that comprises eps gene cluster genes/sequences similar to the novel herein discussed characterizing eps gene cluster genes/sequences of the strains of the invention, would not also have improved texturing properties. In LAB, the Wzy-dependent pathway is the pathway of choice for the synthesis of heteropolymeric EPS.

The genetic loci for polysaccharide biosynthesis by the Wzy-dependent mechanism are similar in all bacteria and are well studied in Streptococcus pneumoniae. Of note, S. pneumoniae produces only capsular exocellular polysaccharides (often abbreviated as CPS), while LAB can produce both CPS and EPS (EPS stands for “exocellular polysaccharide”, which is excreted into the medium/milk). The same gene cluster is responsible for the production of CPS and EPS. Genetic analysis of the CPS locus from 90 pneumococcal serotypes demonstrated a striking feature of the polysaccharide operon: the presence of many highly divergent forms of each of the key enzyme classes. Thus, there were found 40 homology groups for polysaccharide polymerases, 13 groups of lipases, and a great diversity of glycosyltransferases. The presence of multiple non-homologous or highly divergent forms of these enzymes, together with often different G+C content of the region in which these are encoded, supports the view that these genes have been imported on multiple occasions from different and unknown sources. Many eps gene clusters have undergone rearrangement mediated by insertion sequence (IS) elements and received genes from other organisms by a horizontal gene transfer. Typical of eps operon organization is the presence of IS elements flanking or within the operon. The plethora of glycosyltransferases observed in the loci for polysaccharide production provides an opportunity to continually generate new strains producing unique EPS by gene shuffling. As EPS show an enormous diversity in monosaccharide building blocks, anomeric configuration, conformation, and stereochemistry, the resulting diversity of EPS structures is uncanny: for instance, two glucose residues can be joined together in 30 different ways. According to Carbohydrate-Active enZymes (CAZy) database (cazy.org), glycosyltransferases are currently classified into 107 families (June 2019, www.cazy.org/GlycosylTransferases.html), which can help in predicting their mode of action. Nevertheless, this does not mean that all enzymes of a family recognize the same donor and acceptor, as polyspecificity is common among glycosyltransferase families, and thus one should be prudent with the over-interpretation of predictions based purely on this classification.

Genes encoding Wzy-dependent exocellular polysaccharide biosynthesis proteins in LAB are typically organized in a cluster with an operon structure and are generally chromosomal in Streptococcus thermophilus, but can reside on a plasmid or the chromosome in L. lactic and Lactobacillus sp. Generally, eps gene clusters are highly diverse, and their nucleotide sequences are among the most variable sequences in LAB genomes. However, the modular gene organization in eps gene clusters is conserved (Zeidan et al., 2017). The conserved genes in the beginning of the eps gene cluster, which are involved in the modulation and assembly machinery of polysaccharide biosynthesis, were denominated epsRXCDB, according to the nomenclature by Zeidan et al. (2017) and Poulsen et al. (2019), and those at the end, epsL and lytR, while the polymerase was named wzy, and the flippase, wzx. The genes of the variable part include polymerase wzy, polysaccharide transporter also called flippase wzx, and glucosyltransferases (GT) or other polymer-modifying enzymes. The common denominator for the texturing strains is that they all contain the genes required for the polysaccharide production, e.g. epsCDBE-wzy-wzx and GT (Zeidan et al., 2017).

No putative function could be yet assigned to epsX and epsL. NIZO B40 epsL can be disrupted by single crossover using an internal gene fragment or overproduced without any effect on EPS production (van Kranenburg, 1999). However, it might be that the second copy of epsL takes over, if the one from the eps cluster is not functional.

EpsR is believed to be responsible for EPS biosynthesis regulation, and thus certain mutations would affect the EPS production. EpsCDB and ATP are believed to form a stable complex acting as a tyrosine kinase—phosphatase system, which controls EPS synthesis, likely through the phosphorylation of epsE, a glycosylphospho-transferase that catalyzes the first step in the assembly of the EPS repeat unit and defines the type of sugar added to the lipid carrier for the formation of EPS. All three genes responsible for tyrosine phosphorylation are essential for the complete encapsulation of the pneumococcus, with cpsC (corresponding to EpsC in L. lactis) being a major virulence factor, crucial via its role in the regulation of the CPS biosynthesis (Whittall et al., 2015). In L. lactis, epsC and epsD were found to be essential for the EPS production, while epsB was not strictly required, as the effect of its deletion was the reduced amount of EPS produced (Nierop Groot and Kleerebezem, 2007). Gene epsE encoding the initial glycose phosphate transferase, which does not catalyze glycosidic linkage, but is involved in linking the first sugar of the repeat unit to the lipid carrier, was shown to be essential for polysaccharide biosynthesis in L. lactis, as its disruption abolished EPS production (Dabour and LaPointe 2005, van Kranenburg et al., 1997).

Subsequently, the following genes of the eps cluster typically encoding glycosyltransferases, polymerases and transporters, are situated in a variable part of the cluster and do often have a low degree of similarity to already characterized genes, which makes the prediction of their putative functions difficult. Comparison of polysaccharide synthesis operons from 90 pneumococcal serotypes, where polysaccharide biosynthesis is well studied, revealed that central genes responsible for the synthesis and polymerization of the repeat unit are highly variable and often non-homologous between serotypes (Bentley et al., 2006). Wzy-dependent CPS biosynthesis in S. pneumoniae resembles peptidoglycan synthesis, whereby repeat units are built on the inner face of the cytoplasmic membrane, transported to the outer face of the membrane by a Wzx transporter, also called flippase, and polymerized by a Wzy polymerase. The polysaccharide polymerase wzy links individual repeat units to form lipid-linked CPS. In S. pneumonia, 40 homology groups for polysaccharide polymerases were found. The initial sugar of the repeat oligosaccharide unit is also the donor sugar in the polymerization of the repeat units, and the specificity of the Wzy polymerase determines the linkage type. The predictions for initial sugars, and subsequent repeat-unit polymerization linkage, correlate well with the polymerase homology groups. In S. pneumonia, there are 32 polymerase homology groups associated with WchA, five with WciI, four with WcjG and one with WcjH. These associations are mostly exclusive, with only five polymerase homology groups associated with two initial transferases, which indicates a high specificity of the initial transferases (Bentley et al., 2006).

Accordingly, generally, the eps gene clusters of Lactococcus sp. strains have high similarity within the conserved regions (e.g., epsRXCDBE, epsL and lytR), but the remaining part of the eps genes, including wzy, wzx, the GT genes and other oligosaccharide repeating unit modifying genes, if present, are generally more variable, both in terms of sequence and in terms of genes present. Without being limited to theory, it is currently believed that differences in the genes related to EPS biosynthesis of the variable region (in particular the wzy, wzx and the GT genes, if present) are likely to be responsible for the different EPS structures produced by the different LAB strains, which may have an impact on the differences in the texturing capability of the different LAB strains.

As discussed above, a first aspect of the present invention relates to a Lactococcus lactic lactic acid bacterium (LAB) strain comprising an active eps gene cluster capable of producing exopolysaccharide (EPS), wherein the eps gene cluster comprises at least one, preferably two, more preferably three, even more preferably all of nucleotide sequence selected from the nucleotide sequences (a)-(m), as the case may be, see below, as defined in any one of (i) to (x).

In a preferred embodiment, the eps gene cluster comprises all of the nucleotide sequences (a)-(c) (i.e., (a) to (c3)/(c4)/(c6), as the case may be, see below) as defined in any one of (i) to (x), see below.

In the most preferred embodiment, the eps gene cluster comprises all of the nucleotide sequences (a)-(m), as the case may be, (e.g., (a), (b), (c1), (c2), (c3) and (d) for the LAB as defined in (i), (a), (b), (c1), (c2), (c3), (c3), (c4), (c5), (d), (e), (f), (g), (h) and (i) for the LAB as defined in (ii), (a), (b), (c1), (c2), (c3) and (d) for the LAB as defined in (iii), (a), (b), (c1), (c2), (c3), (c4) and (d) for the LAB as defined in (iv), (a), (c1), (c2), (c3), (c4), (d), (e), (f), (g), (h), (i), and (j) for the LAB as defined in (v), (a), (b), (c1), (c2), (c3), (c4), (c5), (c6), (d), (e), (f), (g), (h), (i), (j), (k), (l) and (m) for the LAB as defined in (vi), (a), (c1) (c2) and (d) for the LAB as defined in (vii), (a), (b), (c1), (c2), (c3), (c4), (c5), (c6), (d), (e) and (f) for the LAB as defined in (viii), (a), (b), (c1), (c2), (c3), (d) and (e) for the LAB as defined in (ix) and (a), (b), (c1), (c2), (c3), (d) and (e) for the LAB as defined in (x)), as defined in any one of (i) to (x):
(i) (a): a nucleotide sequence encoding a polypeptide having polymerase activity and having at least 70%, preferably at least 85%, more preferably at least 95%, even more preferably at least 98%, and most preferably 100% identity with the amino acid sequence encoded by the nucleotide sequence of SEQ ID NO: 11 (herein termed wzy);
   (b): a nucleotide sequence encoding a polypeptide having polysaccharide transporter activity and having at least 70%, preferably at least 85%, more preferably at least 95%, even more preferably at least 98%, and most preferably 100% identity with the amino acid sequence encoded by the nucleotide sequence of SEQ ID NO: 17 (herein termed wzx);
   (c): at least one, preferably two, and most preferably three nucleotide sequence(s) encoding a polypeptide having glycosyltransferase (GT) activity selected from:
   (c1): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 9 (herein termed GT1);
   (c2): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 13 (herein termed GT2); and
   (c3): a nucleotide sequence having at least 70%, preferably at least 85%, more preferably at least 95%, even more preferably at least 98%, and most preferably 100% with the amino acid sequence encoded by the nucleotide sequence of SEQ ID NO: 15 (herein termed GT3);
   (d): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 23 (herein termed putative nucleotide sugar dehydrogenase protein);
(ii) (a): a nucleotide sequence encoding a polypeptide having polymerase activity and having at least 70%, preferably at least 85%, more preferably at least 95%, even more preferably at least 98%, and most preferably 100% identity with the amino acid sequence encoded by nucleotides 13939-15042 of SEQ ID NO:199 (herein termed wzy);
   (b): a nucleotide sequence encoding a polypeptide having polysaccharide transporter activity and having at least 70%, preferably at least 85%, more preferably at least 95%, even more preferably at least 98%, and most preferably 100% identity with the amino acid sequence encoded by nucleotides 26029-27444 of the complementary strand of SEQ ID NO:199 (herein termed wzx);
   (c): at least one, preferably two, and more preferably three, even more preferably four and most preferably five nucleotide sequence(s) encoding a polypeptide having glycosyltransferase (GT) activity selected from:
   (c1): a nucleotide sequence having at least 70%, preferably at least 85%, more preferably at least 95%, even more preferably at least 98%, and most preferably 100% identity with the amino acid sequence encoded by nucleotides 4174-4824 of SEQ ID NO:199 (herein termed GT1);
   (c2): a nucleotide sequence having at least 70%, preferably at least 85%, more preferably at least 95%, even more preferably at least 98%, and most preferably 100% identity with the amino acid sequence encoded by nucleotides 7276-8508 of SEQ ID NO:199 (herein termed GT2);
   (c3): a nucleotide sequence having at least 70%, preferably at least 85%, more preferably at least 95%, even more preferably at least 98%, and most preferably 100% identity with the amino acid sequence encoded by nucleotides 11042-12391 of SEQ ID NO:199 (herein termed GT3);
   (c4): a nucleotide sequence having at least 70%, preferably at least 85%, more preferably at least 95%, even more preferably at least 98%, and most preferably 100% identity with the amino acid sequence encoded by nucleotides 13008-13934 of SEQ ID NO:199 (herein termed GT4);
   (c5): a nucleotide sequence having at least 70%, preferably at least 85%, more preferably at least 95%, even more preferably at least 98%, and most preferably 100% identity with the amino acid sequence encoded by nucleotides 18528-19508 of SEQ ID NO:199 (herein termed GT5);
   (d): a nucleotide sequence encoding a polypeptide having dTDP-glucose 4,6-dehydratase activity and having at least 70%, preferably at least 85%, more preferably at least 95%, even more preferably at least 98%, and most preferably 100% identity with the amino acid sequence encoded by nucleotides 4784-5695 of SEQ ID NO:199 (herein termed dTDP-glucose 4,6-dehydratase);
   (e): a nucleotide sequence encoding a polypeptide having dTDP-4-dehydrorhamnose reductase activity and having at least 70%, preferably at least 85%, more preferably at least 95%, even more preferably at least 98%, and most preferably 100% identity with the amino acid sequence encoded by nucleotides 5717-6631 of SEQ ID NO:199 (herein termed dTDP-4-dehydrorhamnose reductase);
   (f): a nucleotide sequence encoding a polypeptide having dTDP-4-dehydrorhamnose 3,5-epimerase activity and having at least 70%, preferably at least 85%, more preferably at least 95%, even more preferably at least 98%, and most preferably 100% identity with the amino acid sequence encoded by nucleotides 6586-7257 of SEQ ID NO:199 (herein termed dTDP-4-dehydrorhamnose 3,5-epimerase);
   (g): a nucleotide sequence encoding polypeptide DUF1919 and having at least 70%, preferably at least 85%, more preferably at least 95%, even more preferably at least 98%, and most preferably 100% identity with the amino acid sequence encoded by nucleotides 8515-9144 of SEQ ID NO:199 (herein termed DUF1919);
   (h): a nucleotide sequence encoding polypeptide DUF4422 and having at least 70%, preferably at least 85%, more preferably at least 95%, even more preferably at least 98%, and most preferably 100% identity with the amino acid sequence encoded by nucleotides 10271-11029 of SEQ ID NO:199 (herein termed DUF4422); and
   (i): a nucleotide sequence encoding a polypeptide having UDP-galactopyranose mutase activity and having at least 70%, preferably at least 85%, more preferably at least 95%, even more preferably at least 98%, and most preferably 100% identity with the amino acid sequence encoded by nucleotides 9159-10274 of SEQ ID NO:199 (herein termed UDP-galactopyranose mutase);
(iii) (a): a nucleotide sequence encoding a polypeptide having polymerase activity and having at least 70%, preferably at least 85%, more preferably at least 95%, even more preferably at least 98%, and most preferably 100% identity with the amino acid sequence encoded by the nucleotide sequence of SEQ ID NO: 39 (herein termed wzy);
   (b): a nucleotide sequence encoding a polypeptide having polysaccharide transporter activity and having at least 70%, preferably at least 85%, more preferably at least 95%, even more preferably at least 98%, and most preferably 100% identity with the amino acid sequence encoded by the nucleotide sequence of SEQ ID NO: 45 (herein termed wzx);
   (c): at least one, preferably two, and most preferably three nucleotide sequence(s) encoding a polypeptide having glycosyltransferase (GT) activity selected from:
   (c1): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 37 (herein termed GT1);
   (c2): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 41 (herein termed GT2); and
   (c3): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 43 (herein termed GT3); and
   (d): a nucleotide sequence encoding a polypeptide having polysaccharide pyruvyl transferase activity and having at least 70%, preferably at least 85%, more preferably at least 95%, even more preferably at least 98%, and most preferably 100% identity with the amino acid sequence encoded by the nucleotide sequence of SEQ ID NO: 51 (herein termed polysaccharide pyruvyl transferase family protein);
(iv) (a): a nucleotide sequence encoding a polypeptide having polymerase activity and having at least 70%, preferably at least 85%, more preferably at least 95%, even more preferably at least 98%, and most preferably 100% identity with the amino acid sequence encoded by the nucleotide sequence of SEQ ID NO: 163 (herein termed wzy);
   (b): a nucleotide sequence encoding a polypeptide having polysaccharide transporter activity and having at least 70%, preferably at least 85%, more preferably at least 95%, even more preferably at least 98%, and most preferably 100% identity with the amino acid sequence encoded by the nucleotide sequence of SEQ ID NO: 169 (herein termed wzx);
   (c): at least one, preferably two, and most preferably three nucleotide sequence(s) encoding a polypeptide having glycosyltransferase (GT) activity selected from:
   (c1): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 161 (herein termed GT1);
   (c2): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 165 (herein termed GT2);
   (c3): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 167 (herein termed GT3); and
   (c4) a nucleotide sequence having at least 70%, preferably at least 85%, more preferably at least 95%, even more preferably at least 98%, and most preferably 100% identity with the amino acid sequence encoded by the nucleotide sequence of SEQ ID NO: 181 (herein termed GT4);
   (d) a nucleotide sequence encoding a polypeptide having at least 70%, preferably at least 85%, more preferably at least 95%, even more preferably at least 98%, and most preferably 100% identity with the amino acid sequence encoded by the nucleotide sequence of SEQ ID NO: 175 (herein termed Core-2/I-Branching protein);
(v) (a): a nucleotide sequence encoding a polypeptide having polymerase activity and having at least 70%, preferably at least 85%, more preferably at least 95%, even more preferably at least 98%, and most preferably 100% identity with the amino acid sequence encoded by nucleotides 13939-15042 of SEQ ID NO: 224 (herein termed wzy);
   (c): at least one, preferably two, and most preferably three nucleotide sequence(s) encoding a polypeptide having glycosyltransferase (GT) activity selected from:
   (c1): a nucleotide sequence having at least 70%, preferably at least 85%, more preferably at least 95%, even more preferably at least 98%, and most preferably 100% identity with the amino acid sequence encoded by nucleotides 4174-4824 of SEQ ID NO: 224 (herein termed GT1);
   (c2): a nucleotide sequence having at least 70%, preferably at least 85%, more preferably at least 95%, even more preferably at least 98%, and most preferably 100% identity with the amino acid sequence encoded by nucleotides 11042-12391 of SEQ ID NO: 224 (herein termed GT2);
   (c3): a nucleotide sequence having at least 70%, preferably at least 85%, more preferably at least 95%, even more preferably at least 98%, and most preferably 100% identity with the amino acid sequence encoded by nucleotides 13008-13934 of SEQ ID NO: 224 (herein termed GT3); and
   (c4): a nucleotide sequence having at least 70%, preferably at least 85%, more preferably at least 95%, even more preferably at least 98%, and most preferably 100% identity with the amino acid sequence encoded by nucleotides 18527-19507 of SEQ ID NO: 224 (herein termed GT4);
   (d): a nucleotide sequence encoding polypeptide DUF1972, and having at least 70%, preferably at least 85%, more preferably at least 95%, even more preferably at least 98%, and most preferably 100% identity with the amino acid sequence encoded by nucleotides 7276-8508 of SEQ ID NO: 224 (herein termed DUF1972);
   (e): a nucleotide sequence encoding polypeptide DUF4422 and having at least 70%, preferably at least 85%, more preferably at least 95%, even more preferably at least 98%, and most preferably 100% identity with the amino acid sequence encoded by nucleotides 10271-11029 of SEQ ID NO: 224 (herein termed DUF4422);
   (f): a nucleotide sequence encoding polypeptide DUF1919 and having at least 70%, preferably at least 85%, more preferably at least 95%, even more preferably at least 98%, and most preferably 100% identity with the amino acid sequence encoded by nucleotides 8515-9144 of SEQ ID NO: 224 (herein termed DUF1919);
   (g): a nucleotide sequence encoding a polypeptide having UDP-galactopyranose mutase activity and having at least 70%, preferably at least 85%, more preferably at least 95%, even more preferably at least 98%, and most preferably 100% identity with the amino acid sequence encoded by nucleotides 9159-10274 of SEQ ID NO: 224 (herein termed UDP-galactopyranose mutase);
   (h): a nucleotide sequence encoding a polypeptide having dTDP-4-dehydrorhamnose 3,5-epimerase activity and having at least 70%, preferably at least 85%, more preferably at least 95%, even more preferably at least 98%, and most preferably 100% identity with the amino acid sequence encoded by nucleotides 6586-7257 of SEQ ID NO: 224 (herein termed dTDP-4-dehydrorhamnose 3,5-epimerase);
   (i): a nucleotide sequence encoding a polypeptide having dTDP-glucose 4,6-dehydratase activity and having at least 70%, preferably at least 85%, more preferably at least 95%, even more preferably at least 98%, and most preferably 100% identity with the amino acid sequence encoded by nucleotides 4784-5695 of SEQ ID NO: 224 (herein termed dTDP-glucose 4,6-dehydratase); and
   (j): a nucleotide sequence encoding a polypeptide having dTDP-4-dehydrorhamnose reductase activity and having at least 70%, preferably at least 85%, more preferably at least 95%, even more preferably at least 98%, and most preferably 100% identity with the amino acid sequence encoded by nucleotides 5717-6631 of SEQ ID NO: 224 (herein termed dTDP-4-dehydrorhamnose reductase);
(vi) (a): a nucleotide sequence encoding a polypeptide having polymerase activity and having at least 95% identity with the amino acid sequence encoded by the nucleotide sequence of SEQ ID NO: 67 (herein termed wzy);
   (b): a nucleotide sequence encoding a polypeptide having polysaccharide transporter activity and having at least 70%, preferably at least 85%, more preferably at least 95%, even more preferably at least 98%, and most preferably 100% identity with the amino acid sequence encoded by the nucleotide sequence of SEQ ID NO: 73 (herein termed wzx);
   (c): at least one, preferably two, and most preferably three nucleotide sequence(s) encoding a polypeptide having glycosyltransferase (GT) activity selected from:
   (c1): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 65 (herein termed GT1);
   (c2): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 69 (herein termed GT2);
   (c3): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 71 (herein termed GT3);
   (c4): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 85 (herein termed GT4);
   (c5): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 87 (herein termed GT5); and
   (c6): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 89 (herein termed GT6);
   (d): a nucleotide sequence having epimerase/dehydratase activity and having at least 70%, preferably at least 85%, more preferably at least 95%, even more preferably at least 98%, and most preferably 100% identity with the amino acid sequence encoded by the nucleotide sequence of SEQ ID NO: 93 (herein termed NAD-dependent epimerase/dehydratase 1);
   (e): a nucleotide sequence having dehydrogenase activity and having at least 70%, preferably at least 85%, more preferably at least 95%, even more preferably at least 98%, and most preferably 100% identity with the amino acid sequence encoded by the nucleotide sequence of SEQ ID NO: 79 (herein termed nucleotide sugar dehydrogenase);
   (f): a nucleotide sequence having thymidylyltransferase activity and having at least 70%, preferably at least 85%, more preferably at least 95%, even more preferably at least 98%, and most preferably 100% identity with the amino acid sequence encoded by the nucleotide sequence of SEQ ID NO: 95 (herein termed rfbA, glucose-1-phosphate thymidylyltransferase);
   (g): a nucleotide sequence having dehydratase activity and having at least 70%, preferably at least 85%, more preferably at least 95%, even more preferably at least 98%, and most preferably 100% identity with the amino acid sequence encoded by the nucleotide sequence of SEQ ID NO: 97 (herein termed dTDP-glucose 4,6-dehydratase);
   (h): a nucleotide sequence having epimerase activity and having at least 70%, preferably at least 85%, more preferably at least 95%, even more preferably at least 98%, and most preferably 100% identity with the amino acid sequence encoded by the nucleotide sequence of SEQ ID NO: 99 (herein termed dTDP-4-dehydrorhamnose 3,5-epimerase);
   (i): a nucleotide sequence having epimerase/dehydratase activity and having at least 70%, preferably at least 85%, more preferably at least 95%, even more preferably at least 98%, and most preferably 100% identity with the amino acid sequence encoded by the nucleotide sequence of SEQ ID NO: 101 (herein termed NAD-dependent epimerase/dehydratase family protein 2);
   (j): a nucleotide sequence having acyltransferase activity and having at least 70%, preferably at least 85%, more preferably at least 95%, even more preferably at least 98%, and most preferably 100% identity with the amino acid sequence encoded by the nucleotide sequence of SEQ ID NO: 111 (herein termed acyltransferase 1);
   (k): a nucleotide sequence having acyltransferase activity and having at least 70%, preferably at least 85%, more preferably at least 95%, even more preferably at least 98%, and most preferably 100% identity with the amino acid sequence encoded by the nucleotide sequence of SEQ ID NO: 107 (herein termed acyltransferase 2);
   (l): a nucleotide sequence having reductase activity and having at least 70%, preferably at least 85%, more preferably at least 95%, even more preferably at least 98%, and most preferably 100% identity with the amino acid sequence encoded by the nucleotide sequence of SEQ ID NO: 103 (herein termed dTDP-4-dehydrorhamnose reductase); and
   (m): a nucleotide sequence having nucleotidyl transferase activity and having at least 70%, preferably at least 85%, more preferably at least 95%, even more preferably at least 98%, and most preferably 100% identity with the amino acid sequence encoded by the nucleotide sequence of SEQ ID NO: 105 (herein termed nucleotidyl transferase);
(vii) (a): a nucleotide sequence encoding a polypeptide having polymerase activity and having at least 70%, preferably at least 85%, more preferably at least 95%, even more preferably at least 98%, and most preferably 100% identity with the amino acid sequence encoded by nucleotides 5833-6927 of SEQ ID NO: 244 (herein termed wzy);
   (c): at least one, preferably two, and most preferably three nucleotide sequence(s) encoding a polypeptide having glycosyltransferase (GT) activity selected from:
   (c1): a nucleotide sequence having at least 70%, preferably at least 85%, more preferably at least 95%, even more preferably at least 98%, and most preferably 100% identity with the amino acid sequence encoded by nucleotides 4617-5123 of SEQ ID NO: 244 (herein termed GT1); and
   (c2): a nucleotide sequence having at least 70%, preferably at least 85%, more preferably at least 95%, even more preferably at least 98%, and most preferably 100% identity with the amino acid sequence encoded by nucleotides 5120-5827 of SEQ ID NO: 244 (herein termed GT2); and
   (d): a nucleotide sequence encoding a polypeptide having UDP-N-acetylglucosamine-LPS N-acetylglucosamine transferase activity and having at least 70%, preferably at least 85%, more preferably at least 95%, even more preferably at least 98%, and most preferably 100% identity with the amino acid sequence encoded by nucleotides 4168-4617 of SEQ ID NO: 244 (herein termed UDP-N-acetylglucosamine-LPS N-acetylglucosa mine transferase);
(viii) (a): a nucleotide sequence encoding a polypeptide having polymerase activity and having at least 70%, preferably at least 85%, more preferably at least 95%, even more preferably at least 98%, and most preferably 100% identity with the amino acid sequence encoded by the nucleotide sequence of SEQ ID NO: 123 (herein termed wzy);
   (b): a nucleotide sequence encoding a polypeptide having polysaccharide transporter activity and having at least 70%, preferably at least 85%, more preferably at least 95%, even more preferably at least 98%, and most preferably 100% identity with the amino acid sequence encoded by the nucleotide sequence of SEQ ID NO: 129 (herein termed wzx);
   (c): at least one, preferably two, and most preferably three nucleotide sequence(s) encoding a polypeptide having glycosyltransferase (GT) activity selected from:
   (c1): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 121 (herein termed GT1);
   (c2): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 125 (herein termed GT2);
   (c3): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 127 (herein termed GT3);
   (c4): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 143 (herein termed GT4);
   (c5): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 145 (herein termed GT5); and
   (c6): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 147 (herein termed GT6);
   (d): a nucleotide sequence encoding a polypeptide having acetyltransferase activity and having at least 70%, preferably at least 85%, more preferably at least 95%, even more preferably at least 98%, and most preferably 100% identity with the amino acid sequence encoded by the nucleotide sequence of SEQ ID NO: 149 (herein termed acetyltransferase);
   (e): a nucleotide sequence encoding a polypeptide having dehydrogenase activity and having at least 70%, preferably at least 85%, more preferably at least 95%, even more preferably at least 98%, and most preferably 100% identity with the amino acid sequence encoded by the nucleotide sequence of SEQ ID NO: 135 (herein termed nucleotide sugar dehydrogenase);
   (f): a nucleotide sequence encoding a polypeptide having acyltransferase activity and having at least 70%, preferably at least 85%, more preferably at least 95%, even more preferably at least 98%, and most preferably 100% identity with the amino acid sequence encoded by the nucleotide sequence of SEQ ID NO: 151 (herein termed acyltransferase);
(ix) (a): a nucleotide sequence encoding a polypeptide having polymerase activity and having at least 70%, preferably at least 85%, more preferably at least 95%, even more preferably at least 98%, and most preferably 100% identity with the amino acid sequence encoded by nucleotides 11201-12349 of the complementary strand of SEQ ID NO: 257 (herein termed wzy);
   (b): a nucleotide sequence encoding a polypeptide having polysaccharide transporter activity and having at least 70%, preferably at least 85%, more preferably at least 95%, even more preferably at least 98%, and most preferably 100% identity with the amino acid sequence encoded by nucleotides 15538-16953 of the complementary strand of SEQ ID NO: 257 (herein termed wzx);
   (c): a nucleotide sequence encoding a polypeptide having glycosyltransferase (GT) activity comprising:
   (c1): a nucleotide sequence having at least 70%, preferably at least 85%, more preferably at least 95%, even more preferably at least 98%, and most preferably 100% identity with the amino acid sequence encoded by nucleotides 9726-10673 of the complementary strand of SEQ ID NO: 257 (herein termed GT1);
   (c2): a nucleotide sequence having at least 70%, preferably at least 85%, more preferably at least 95%, even more preferably at least 98%, and most preferably 100% identity with the amino acid sequence encoded by nucleotides 12336-13421 of the complementary strand of SEQ ID NO: 257 (herein termed GT2); and
   (c3): a nucleotide sequence having at least 70%, preferably at least 85%, more preferably at least 95%, even more preferably at least 98%, and most preferably 100% identity with the amino acid sequence encoded by nucleotides 13418-14260 of the complementary strand of SEQ ID NO: 257 (herein termed GT3);
   (d): a nucleotide sequence encoding a polypeptide having nucleotide sugar dehydrogenase activity and having at least 70%, preferably at least 85%, more preferably at least 95%, even more preferably at least 98%, and most preferably 100% identity with the amino acid sequence encoded by nucleotides 7727-8173 of the complementary strand of SEQ ID NO: 257 (herein termed nucleotide sugar dehydrogenase); and
   (e): a nucleotide sequence encoding a polypeptide having acetyltransferase activity and having at least 70%, preferably at least 85%, more preferably at least 95%, even more preferably at least 98%, and most preferably 100% identity with the amino acid sequence encoded by nucleotides 10657-11211 of the complementary strand of SEQ ID NO: 257 (herein termed acetyltransferase);
(x) a): a nucleotide sequence encoding a polypeptide having polymerase activity and having at least 70%, preferably at least 85%, more preferably at least 95%, even more preferably at least 98%, and most preferably 100% identity with the amino acid sequence encoded by nucleotides 10707-11846 of the complementary strand of SEQ ID NO: 274 (herein termed wzy);
   (b): a nucleotide sequence encoding a polypeptide having polysaccharide transporter activity and having at least 70%, preferably at least 85%, more preferably at least 95%, even more preferably at least 98%, and most preferably 100% identity with the amino acid sequence encoded by nucleotides 15037-16476 of the complementary strand of SEQ ID NO: 274 (herein termed wzx);
   (c): a nucleotide sequence encoding a polypeptide having glycosyltransferase (GT) activity comprising:
   (c1): a nucleotide sequence having at least 70%, preferably at least 85%, more preferably at least 95%, even more preferably at least 98%, and most preferably 100% identity with the amino acid sequence encoded by nucleotides 9232-10179 of the complementary strand of SEQ ID NO: 274 (herein termed GT1);
   (c2): a nucleotide sequence having at least 70%, preferably at least 85%, more preferably at least 95%, even more preferably at least 98%, and most preferably 100% identity with the amino acid sequence encoded by nucleotides 11833-12918 of the complementary strand of SEQ ID NO: 274 (herein termed GT2); and
   (c3): a nucleotide sequence having at least 70%, preferably at least 85%, more preferably at least 95%, even more preferably at least 98%, and most preferably 100% identity with the amino acid sequence encoded by nucleotides 12915-13757 of the complementary strand of SEQ ID NO: 274 (herein termed GT3);
   (d): a nucleotide sequence encoding a polypeptide having nucleotide sugar dehydrogenase activity and having at least 70%, preferably at least 85%, more preferably at least 95%, even more preferably at least 98%, and most preferably 100% identity with the amino acid sequence encoded by nucleotides 7234-7680 of the complementary strand of SEQ ID NO: 274 (herein termed nucleotide sugar dehydrogenase); and
   (e): a nucleotide sequence encoding a polypeptide having acetyltransferase activity and having at least 70%, preferably at least 85%, more preferably at least 95%, even more preferably at least 98%, and most preferably 100% identity with the amino acid sequence encoded by nucleotides 10163-10717 of the complementary strand of SEQ ID NO: 274 (herein termed acetyltransferase).

The above sequences are all present in the variable part of the eps gene cluster of the strains of the present invention.

Accordingly, in a preferred embodiment, the Lactococcus lactis lactic acid bacterium (LAB) strain of the present invention comprises an active eps gene cluster capable of producing exopolysaccharide (EPS), wherein the eps gene cluster comprises all nucleotide sequences (a)-(c), if present, see above, as defined in any one of (i) to (x) above.

In the most preferred embodiment, the Lactococcus lactis lactic acid bacterium (LAB) strain of the present invention comprises an active eps gene cluster capable of producing exopolysaccharide (EPS), wherein the eps gene cluster comprises all nucleotide sequences (a)-(m), as the case may be, see above, as defined in any one of (i) to (x) above.

Preferably, the Lactococcus lactis lactic acid bacterium (LAB) strain of the present invention comprises an active eps gene cluster capable of producing exopolysaccharide (EPS), preferably wherein the eps gene cluster comprises all nucleotide sequences (a)-(c), as the case may be, as defined in any one of (i) to (x) above, even more preferably wherein the eps gene cluster comprises all nucleotide sequences (a)-(m), as the case may be, as defined in any one of (i) to (x) above and at least one, preferably all of the following nucleotide sequences:
(i) (1): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 1 (herein termed epsR);
   (2): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 3 (herein termed epsX);
   (3): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 5 (herein termed epsB);
   (4): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 7 (herein termed epsD);
   (5): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 19 (herein termed epsL);
   (6): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 21 (herein termed LytR family transcriptional regulator protein);
   (7): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 25 (herein termed epsC);
   (8): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 27 (herein termed epsE);
(ii) (1): a nucleotide sequence having at least 70%, preferably at least 85%, more preferably at least 95%, even more preferably at least 98%, and most preferably 100% identity with the amino acid sequence encoded by nucleotides 1-318 of SEQ ID NO:199 (herein termed epsR);
   (2): a nucleotide sequence having at least 70%, preferably at least 85%, more preferably at least 95%, even more preferably at least 98%, and most preferably 100% identity with the amino acid sequence encoded by nucleotides 352-1119 of SEQ ID NO:199 (herein termed epsX);
   (3): a nucleotide sequence having at least 70%, preferably at least 85%, more preferably at least 95%, even more preferably at least 98%, and most preferably 100% identity with the amino acid sequence encoded by nucleotides 2698 to 3462 of SEQ ID NO:199 (herein termed epsB);
   (4): a nucleotide sequence having at least 70%, preferably at least 85%, more preferably at least 95%, even more preferably at least 98%, and most preferably 100% identity with the amino acid sequence encoded by nucleotides 1948-2640 of SEQ ID NO:199 (herein termed epsD);
   (5): a nucleotide sequence having at least 70%, preferably at least 85%, more preferably at least 95%, even more preferably at least 98%, and most preferably 100% identity with the amino acid sequence encoded by nucleotides 24053-24751 of the complementary strand of SEQ ID NO:199 (herein termed epsL);
   (6): a nucleotide sequence having at least 70%, preferably at least 85%, more preferably at least 95%, even more preferably at least 98%, and most preferably 100% identity with the amino acid sequence encoded by nucleotides 20389-21291 of SEQ ID NO:199 (herein termed LytR family transcriptional regulator protein);
   (7): a nucleotide sequence having at least 70%, preferably at least 85%, more preferably at least 95%, even more preferably at least 98%, and most preferably 100% identity with the amino acid sequence encoded by nucleotides 1159 to 1938 of SEQ ID NO:199 (herein termed epsC);
   (8): a nucleotide sequence having at least 70%, preferably at least 85%, more preferably at least 95%, even more preferably at least 98%, and most preferably 100% identity with the amino acid sequence encoded by nucleotides 3484-4170 of SEQ ID NO:199 (herein termed epsE);
(iii) (1): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 29 (herein termed epsR);
   (2): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 31 (herein termed epsX);
   (3): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 33 (herein termed epsB);
   (4): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 35 (herein termed epsD);
   (5): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 47 (herein termed epsL);
   (6): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 49 (herein termed putative LytR family transcriptional regulator protein);
   (7): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 53 (herein termed epsC);
   (8): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 55 (herein termed epsE);
(iv) (1): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 153 (herein termed epsR);
   (2): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 155 (herein termed epsX);
   (3): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 157 (herein termed epsB);
   (4): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 159 (herein termed epsD);
   (5): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 171 (herein termed epsL);
   (6): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 173 (herein termed putative LytR family transcriptional regulator protein);
   (7): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 177 (herein termed epsC); and
   (8): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 179 (herein termed epsE);
(v) (1): a nucleotide sequence having at least 70%, preferably at least 85%, more preferably at least 95%, even more preferably at least 98%, and most preferably 100% identity with the amino acid sequence encoded by nucleotides 1-318 of SEQ ID NO: 224 (herein termed epsR);
   (2): a nucleotide sequence having at least 70%, preferably at least 85%, more preferably at least 95%, even more preferably at least 98%, and most preferably 100% identity with the amino acid sequence encoded by nucleotides 352-1119 of SEQ ID NO: 224 (herein termed epsX);
   (3): a nucleotide sequence having at least 70%, preferably at least 85%, more preferably at least 95%, even more preferably at least 98%, and most preferably 100% identity with the amino acid sequence encoded by nucleotides 2698-3462 of SEQ ID NO: 224 (herein termed epsB);
   (4): a nucleotide sequence having at least 70%, preferably at least 85%, more preferably at least 95%, even more preferably at least 98%, and most preferably 100% identity with the amino acid sequence encoded by nucleotides 1948-2640 of SEQ ID NO: 224 (herein termed epsD);
   (5): a nucleotide sequence having at least 70%, preferably at least 85%, more preferably at least 95%, even more preferably at least 98%, and most preferably 100% identity with the amino acid sequence encoded by nucleotides 20388-21290 of SEQ ID NO: 224 (herein termed LytR family transcriptional regulator protein);
   (6): a nucleotide sequence having at least 70%, preferably at least 85%, more preferably at least 95%, even more preferably at least 98%, and most preferably 100% identity with the amino acid sequence encoded by nucleotides 1159-1938 of SEQ ID NO: 224 (herein termed epsC);
   (7): a nucleotide sequence having at least 70%, preferably at least 85%, more preferably at least 95%, even more preferably at least 98%, and most preferably 100% identity with the amino acid sequence encoded by nucleotides 3484-4170 of SEQ ID NO: 224 (herein termed epsE);
(vi) (1): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 57 (herein termed epsR);
   (2): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 59 (herein termed epsX);
   (3): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 61 (herein termed epsB);
   (4): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 63 (herein termed epsD);
   (5): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 75 (herein termed epsL);
   (6): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 77 (herein termed putative LytR family transcriptional regulator protein);
   (7): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 81 (herein termed epsC);
   (8): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 83 (herein termed epsE);
(vii) (1): a nucleotide sequence having at least 70%, preferably at least 85%, more preferably at least 95%, even more preferably at least 98%, and most preferably 100% identity with the amino acid sequence encoded by nucleotides 1-318 of SEQ ID NO: 244 (herein termed epsR);
   (2): a nucleotide sequence having at least 70%, preferably at least 85%, more preferably at least 95%, even more preferably at least 98%, and most preferably 100% identity with the amino acid sequence encoded by nucleotides 352-1119 of SEQ ID NO: 244 (herein termed epsX);
   (3): a nucleotide sequence having at least 70%, preferably at least 85%, more preferably at least 95%, even more preferably at least 98%, and most preferably 100% identity with the amino acid sequence encoded by nucleotides 2698-3462 of SEQ ID NO: 244 (herein termed epsB);
   (4): a nucleotide sequence having at least 70%, preferably at least 85%, more preferably at least 95%, even more preferably at least 98%, and most preferably 100% identity with the amino acid sequence encoded by nucleotides 1948-2643 of SEQ ID NO: 244 (herein termed epsD);
   (5): a nucleotide sequence having at least 70%, preferably at least 85%, more preferably at least 95%, even more preferably at least 98%, and most preferably 100% identity with the amino acid sequence encoded by nucleotides 9365-10267 of the complementary strand of SEQ ID NO: 244 (herein termed LytR family transcriptional regulator protein);
   (6): a nucleotide sequence having at least 70%, preferably at least 85%, more preferably at least 95%, even more preferably at least 98%, and most preferably 100% identity with the amino acid sequence encoded by nucleotides 1159-1938 of SEQ ID NO: 244 (herein termed epsC);
   (7): a nucleotide sequence having at least 70%, preferably at least 85%, more preferably at least 95%, even more preferably at least 98%, and most preferably 100% identity with the amino acid sequence encoded by nucleotides 3484-4164 of SEQ ID NO: 244 (herein termed epsE;
   (8): a nucleotide sequence having at least 70%, preferably at least 85%, more preferably at least 95%, even more preferably at least 98%, and most preferably 100% identity with the amino acid sequence encoded by nucleotides 8438-9340 of SEQ ID NO: 244 (herein termed epsL);
(viii) (1): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 113 (herein termed epsR);
   (2): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 115 (herein termed epsX);
   (3): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 117 (herein termed epsB);
   (4): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 119 (herein termed epsD);
   (5): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 131 (herein termed epsL);
   (6): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 133 (herein termed putative LytR family transcriptional regulator protein);
   (7): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 137 (herein termed epsC);
   (8): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 139 (herein termed epsE1);
   (9): a nucleotide sequence having at least 70%, preferably at least 85%, more preferably at least 95%, even more preferably at least 98%, and most preferably 100% identity with the amino acid sequence encoded by the nucleotide sequence of SEQ ID NO: 141 (herein termed epsE2);
(ix) (1): a nucleotide sequence having at least 70%, preferably at least 85%, more preferably at least 95%, even more preferably at least 98%, and most preferably 100% identity with the amino acid sequence encoded by nucleotides 1-318 of SEQ ID NO: 257 (herein termed epsR);
   (2): a nucleotide sequence having at least 70%, preferably at least 85%, more preferably at least 95%, even more preferably at least 98%, and most preferably 100% identity with the amino acid sequence encoded by nucleotides 407-1120 of SEQ ID NO: 257 (herein termed epsX);
   (3): a nucleotide sequence having at least 70%, preferably at least 85%, more preferably at least 95%, even more preferably at least 98%, and most preferably 100% identity with the amino acid sequence encoded by nucleotides 2699-3463 of SEQ ID NO: 257 (herein termed epsB);
   (4): a nucleotide sequence having at least 70%, preferably at least 85%, more preferably at least 95%, even more preferably at least 98%, and most preferably 100% identity with the amino acid sequence encoded by nucleotides 1949-2644 of SEQ ID NO: 257 (herein termed epsD);
   (5): a nucleotide sequence having at least 70%, preferably at least 85%, more preferably at least 95%, even more preferably at least 98%, and most preferably 100% identity with the amino acid sequence encoded by nucleotides 5876-6778 of SEQ ID NO: 257 (herein termed LytR family transcriptional regulator protein);
   (6): a nucleotide sequence having at least 70%, preferably at least 85%, more preferably at least 95%, even more preferably at least 98%, and most preferably 100% identity with the amino acid sequence encoded by nucleotides 1160-1939 of SEQ ID NO: 257 (herein termed epsC);
   (7): a nucleotide sequence having at least 70%, preferably at least 85%, more preferably at least 95%, even more preferably at least 98%, and most preferably 100% identity with the amino acid sequence encoded by nucleotides 3485-4084 of SEQ ID NO: 257 (herein termed epsE1;
   (8): a nucleotide sequence having at least 70%, preferably at least 85%, more preferably at least 95%, even more preferably at least 98%, and most preferably 100% identity with the amino acid sequence encoded by nucleotides 4085-4840 of SEQ ID NO: 257 (herein termed epsE2;
   (9): a nucleotide sequence having at least 70%, preferably at least 85%, more preferably at least 95%, even more preferably at least 98%, and most preferably 100% identity with the amino acid sequence encoded by nucleotides 6803-7717 of the complementary strand of SEQ ID NO: 257 (herein termed epsL);
(x) (1): a nucleotide sequence having at least 70%, preferably at least 85%, more preferably at least 95%, even more preferably at least 98%, and most preferably 100% identity with the amino acid sequence encoded by nucleotides 1-318 of SEQ ID NO: 274 (herein termed epsR);
   (2): a nucleotide sequence having at least 70%, preferably at least 85%, more preferably at least 95%, even more preferably at least 98%, and most preferably 100% identity with the amino acid sequence encoded by nucleotides 2220-3005 of SEQ ID NO: 274 (herein termed epsB);
   (3): a nucleotide sequence having at least 70%, preferably at least 85%, more preferably at least 95%, even more preferably at least 98%, and most preferably 100% identity with the amino acid sequence encoded by nucleotides 1470-2165 of SEQ ID NO: 274 (herein termed epsD);
   (4): a nucleotide sequence having at least 70%, preferably at least 85%, more preferably at least 95%, even more preferably at least 98%, and most preferably 100% identity with the amino acid sequence encoded by nucleotides 5383-6285 of SEQ ID NO: 274 (herein termed LytR family transcriptional regulator protein);
   (5): a nucleotide sequence having at least 70%, preferably at least 85%, more preferably at least 95%, even more preferably at least 98%, and most preferably 100% identity with the amino acid sequence encoded by nucleotides 681-1460 of SEQ ID NO: 274 (herein termed epsC);
   (6): a nucleotide sequence having at least 70%, preferably at least 85%, more preferably at least 95%, even more preferably at least 98%, and most preferably 100% identity with the amino acid sequence encoded by nucleotides 2992-3591 of SEQ ID NO: 274 (herein termed epsE1);
   (7): a nucleotide sequence having at least 70%, preferably at least 85%, more preferably at least 95%, even more preferably at least 98%, and most preferably 100% identity with the amino acid sequence encoded by nucleotides 3592-4347 of SEQ ID NO: 274 (herein termed epsE2;
   (8): a nucleotide sequence having at least 70%, preferably at least 85%, more preferably at least 95%, even more preferably at least 98%, and most preferably 100% identity with the amino acid sequence encoded by nucleotides 6310-7224 of the complementary strand of SEQ ID NO: 274 (herein termed epsL);

The above sequences are all present in the conserved part of the eps gene cluster of the strains of the present invention.

Even more preferably, the Lactococcus lactic lactic acid bacteria of the present invention comprises an active eps gene cluster capable of producing exopolysaccharide (EPS), wherein the eps gene cluster has the following sequence:
(i) SEQ ID NO:91;
(ii) SEQ ID NO:199;
(iii) SEQ ID NO:92;
(iv) SEQ ID NO:223;
(v) SEQ ID NO:224;
(vi) SEQ ID NO:221;
(vii) SEQ ID NO:244;
(viii) SEQ ID NO:222;
(ix) SEQ ID NO:257;
(x) SEQ ID NO:274.

Most preferably, the Lactococcus lactic lactic acid bacteria of the present invention are the following texturing strains:
(i): DSM 33134
(ii): DSM 33135
(iii): DSM 33136
(iv): DSM 33137
(v): DSM 33138
(vi): DSM 33139
(vii): DSM 33140
(viii): DSM 33141
(ix): DSM 33142
(x): DSM 33183.

The eps gene cluster of DSM 33134 comprises the following genes in the variable and conserved parts:
Variable part: wzy (SEQ ID NO.: 11), wzx (SEQ ID NO.: 17), gene coding GT1 (SEQ ID NO.:9), gene coding GT2 (SEQ ID NO.:13) gene coding GT3 (SEQ ID NO.:15) and gene coding a nucleotide sugar dehydrogenase protein (SEQ ID NO.: 23).
Conserved part: epsR (SEQ ID NO.: 1), epsX (SEQ ID NO.: 3), epsB (SEQ ID NO.: 5), epsD (SEQ ID NO.: 7), epsL (SEQ ID NO.: 19), gene coding LytR protein (SEQ ID NO.: 21), epsC (SEQ ID NO.: 25) and epsE (SEQ ID NO.: 27).

The eps gene cluster of DSM 33136 comprises the following genes in the variable and conserved parts:
Variable part: wzy (SEQ ID NO.: 39), wzx (SEQ ID NO.: 45), gene coding GT1 (SEQ ID NO.:37), gene coding GT2 (SEQ ID NO.:41), gene coding GT3 (SEQ ID NO.:43) and gene coding a polysaccharide pyruvyl transferase family protein (SEQ ID NO.: 51).
Conserved part: epsR (SEQ ID NO.: 29), epsX (SEQ ID NO.: 31), epsB (SEQ ID NO.: 33), epsD (SEQ ID NO.: 35), epsL (SEQ ID NO.: 47), gene coding LytR protein (SEQ ID NO.: 49), epsC (SEQ ID NO.: 25) and epsE (SEQ ID NO.: 27).

The eps gene cluster of DSM 33139 comprises the following genes in the variable and conserved parts:
Variable part: gene coding GT1 (SEQ ID NO.:65), gene coding NAD-dependent epimerase/dehydratase protein 1 (SEQ ID NO.:93), gene coding nucleotide sugar dehydrogenase protein (SEQ ID NO.:79), gene coding glucose-1-phosphate thymidylyltransferase RfbA protein (SEQ ID NO.:95), gene coding dTDP-glucose 4,6-dehydratase protein (SEQ ID NO.:97), gene coding dTDP-4-dehydrorhamnose 3,5-epimerase protein (SEQ ID NO.:99), gene coding NAD-dependent epimerase/dehydratase protein 2 (SEQ ID NO.:101), gene coding GT2 (SEQ ID NO.:69), gene coding GT3 (SEQ ID NO.:71), gene coding GT4 (SEQ ID NO.:85), gene coding GT5 (SEQ ID NO.:87), gene coding GT6 (SEQ ID NO.:89), wzy (SEQ ID NO.:67), gene coding acyltransferase protein 1 (SEQ ID NO.:111), wzx (SEQ ID NO.:73), gene coding acyltransferase protein 2 (SEQ ID NO.:107), gene coding dTDP-4-dehydrorhamnose reductase (SEQ ID NO.:103), gene coding nucleotidyl transferase (SEQ ID NO.:105).
Conserved part: epsR (SEQ ID NO.: 57), epsX (SEQ ID NO.: 59), epsB (SEQ ID NO.: 61), epsD (SEQ ID NO.: 63), epsL (SEQ ID NO.: 75), gene coding LytR protein (SEQ ID NO.: 77), epsC (SEQ ID NO.: 81) and epsE (SEQ ID NO.: 83).

The eps gene cluster of DSM 33141 comprises the following genes in the variable and conserved parts:
Variable part: gene coding GT1 (SEQ ID NO.:121), gene coding nucleotide sugar dehydrogenase protein (SEQ ID NO.:135), gene coding GT2 (SEQ ID NO.:125), gene coding GT3 (SEQ ID NO.:127), gene coding GT4 (SEQ ID NO.:143), gene coding GT5 (SEQ ID NO.:145), gene coding GT6 (SEQ ID NO.:147), wzy (SEQ ID NO.:123), gene coding acetyltransferase protein (SEQ ID NO.:149), wzx (SEQ ID NO.:129), gene coding acyltransferase protein (SEQ ID NO.:151).
Conserved part: epsR (SEQ ID NO.: 113), epsX (SEQ ID NO.: 115), epsB (SEQ ID NO.: 117), epsD (SEQ ID NO.: 119), epsL (SEQ ID NO.: 131), gene coding LytR protein (SEQ ID NO.: 133), epsC (SEQ ID NO.: 137), epsE1 (SEQ ID NO.: 139) and epsE2 (SEQ ID NO.: 141).

The eps gene cluster of DSM 33137 comprises the following genes in the variable and conserved parts:
Variable part: gene coding GT1 (SEQ ID NO.:161), gene coding GT2 (SEQ ID NO.:165), gene coding GT3 (SEQ ID NO.:167), gene coding GT4 (SEQ ID NO.:181), wzy (SEQ ID NO.:163), wzx (SEQ ID NO.:169), gene coding Core-2/I-Branching protein (SEQ ID NO.:175).
Conserved part: epsR (SEQ ID NO.: 153), epsX (SEQ ID NO.: 155), epsB (SEQ ID NO.: 157), epsD (SEQ ID NO.: 159), epsL (SEQ ID NO.: 171), gene coding LytR protein (SEQ ID NO.: 173), epsC (SEQ ID NO.: 177) and epsE (SEQ ID NO.: 179).

The eps gene cluster of DSM 33135 comprises the following genes in the variable and conserved parts:
Variable part: gene coding GT1 (nucleotides 4174-4824 of SEQ ID NO:199), gene coding GT2 (nucleotides 7276-8508 of SEQ ID NO:199), gene coding GT3 (nucleotides 11042-12391 of SEQ ID NO:199), gene coding GT4 (nucleotides 13008-13934 of SEQ ID NO:199), gene coding GT5 (nucleotides 18528-19508 of SEQ ID NO:199), wzy (nucleotides 13939-15042 of SEQ ID NO:199), wzx (nucleotides 26029-27444 of the complementary strand of SEQ ID NO:199), gene coding dTDP-glucose 4,6-dehydratase protein (nucleotides 4784-5695 of SEQ ID NO:199), gene coding dTDP-4-dehydrorhamnose reductase protein (nucleotides 5717-6631 of SEQ ID NO:199), gene coding dTDP-4-dehydrorhamnose 3,5-epimerase protein (nucleotides 6586-7257 of SEQ ID NO:199), gene coding DUF1919 protein (nucleotides 8515-9144 of SEQ ID NO:199), gene coding UDP-galactopyranose mutase protein (nucleotides 9159-10274 of SEQ ID NO:199), gene coding DUF4422 protein (nucleotides 10271-11029 of SEQ ID NO:199).
Conserved part: epsR (nucleotides 1-318 of SEQ ID NO:199), epsX (nucleotides 352-1119 of SEQ ID NO:199), epsB (nucleotides 2698 to 3462 of SEQ ID NO:199), epsD (nucleotides 1948-2640 of SEQ ID NO:199), epsL (nucleotides 24053-24751 of the complementary strand of SEQ ID NO:199), gene coding LytR protein (nucleotides 20389-21291 of SEQ ID NO:199), epsC (nucleotides 1159 to 1938 of SEQ ID NO:199) and epsE (nucleotides 3484-4170 of SEQ ID NO:199).

The eps gene cluster of DSM 33138 comprises the following genes in the variable and conserved parts:
Variable part: gene coding GT1 (nucleotides 4174-4824 of SEQ ID NO: 224), gene coding GT2 (nucleotides 11042-12391 of SEQ ID NO: 224), gene coding GT3 (nucleotides 13008-13934 of SEQ ID NO: 224), gene coding GT4 (nucleotides 18527-19507 of SEQ ID NO: 224), wzy (nucleotides 13939-15042 of SEQ ID NO: 224), gene coding DUF1972 protein (nucleotides 7276-8508 of SEQ ID NO: 224), gene coding dTDP-4-dehydrorhamnose reductase protein (nucleotides 5717-6631 of SEQ ID NO:199), gene coding DUF4422 protein (nucleotides 6586-7257 of SEQ ID NO:199), gene coding DUF1919 protein (nucleotides 8515-9144 of SEQ ID NO:199), gene coding UDP-galactopyranose mutase protein (nucleotides 9159-10274 of SEQ ID NO: 224), gene coding DUF4422 protein (nucleotides 10271-11029 of SEQ ID NO: 224), gene coding DUF1919 protein (nucleotides 8515-9144 of SEQ ID NO: 224), gene coding dTDP-4-dehydrorhamnose_3,5-epimerase protein (nucleotides 6586-7257 of SEQ ID NO: 224), gene coding dTDP-glucose 4,6-dehydratase protein (nucleotides 4784-5695 of SEQ ID NO: 224), and gene coding dTDP-4-dehydrorhamnose reductase protein (nucleotides 5717-6631 of SEQ ID NO: 224).
Conserved part: epsR (nucleotides 1-318 of SEQ ID NO: 224), epsX (nucleotides 352-1119 of SEQ ID NO: 224), epsB (nucleotides 2698-3462 of SEQ ID NO: 224), epsD (nucleotides 1948-2640 of SEQ ID NO: 224), gene coding LytR protein (nucleotides 20388-21290 of SEQ ID NO: 224), epsC (nucleotides 1159-1938 of SEQ ID NO: 224) and epsE (nucleotides 3484-4170 of SEQ ID NO: 224).

The eps gene cluster of DSM 33140 comprises the following genes in the variable and conserved parts:
Variable part: gene coding GT1 (nucleotides 4617-5123 of SEQ ID NO: 244), gene coding GT2 (nucleotides 5120-5827 of SEQ ID NO: 244), wzy (nucleotides 5833-6927 of SEQ ID NO: 244), gene coding dTDP-4-dehydrorhamnose reductase protein (nucleotides 5717-6631 of SEQ ID NO:199), gene coding UDP-N-acetylglucosamine-LPS N-acetylglucosannine_transferaseprotein (nucleotides 4168-4617 of SEQ ID NO: 244).
Conserved part: epsR (nucleotides 1-318 of SEQ ID NO: 244), epsX (nucleotides 352-1119 of SEQ ID NO: 244), epsB (nucleotides 2698-3462 of SEQ ID NO: 244), epsD (nucleotides 1948-2643 of SEQ ID NO: 244), epsL (nucleotides 8438-9340 of SEQ ID NO: 244), gene coding LytR protein (nucleotides 9365-10267 of the complementary strand of SEQ ID NO: 244), epsC (nucleotides 1159-1938 of SEQ ID NO: 244) and epsE (nucleotides 3484-4164 of SEQ ID NO: 244).

The eps gene cluster of DSM 33142 comprises the following genes in the variable and conserved parts:
Variable part: gene coding GT1 (nucleotides 9726-10673 of the complementary strand of SEQ ID NO: 257), gene coding GT2 (nucleotides 12336-13421 of the complementary strand of SEQ ID NO: 257), gene coding GT3 (nucleotides 13418-14260 of the complementary strand of SEQ ID NO: 257), wzy (nucleotides 11201-12349 of the complementary strand of SEQ ID NO: 257), wzx (nucleotides 15538-16953 of the complementary strand of SEQ ID NO: 257), gene coding nucleotide sugar dehydrogenase protein (nucleotides 7727-8173 of the complementary strand of SEQ ID NO: 257) and gene coding acetyltransferase protein (nucleotides 10657-11211 of the complementary strand of SEQ ID NO: 257).
Conserved part: epsR (nucleotides 1-318 of SEQ ID NO: 257), epsX (nucleotides 407-1120 of SEQ ID NO: 257), epsB (nucleotides 2699-3463 of SEQ ID NO: 257), epsD (nucleotides 1949-2644 of SEQ ID NO: 257), epsL (nucleotides 6803-7717 of the complementary strand of SEQ ID NO: 257), gene coding LytR protein (nucleotides 5876-6778 of SEQ ID NO: 257), epsC (nucleotides 1160-1939 of SEQ ID NO: 257), epsE1 (nucleotides 3485-4084 of SEQ ID NO: 257) and epsE2 (nucleotides 4085-4840 of SEQ ID NO: 257).

The eps gene cluster of DSM 33183 comprises the following genes in the variable and conserved parts:
Variable part: gene coding GT1 (nucleotides 9232-10179 of the complementary strand of SEQ ID NO: 274), gene coding GT2 (nucleotides 11833-12918 of the complementary strand of SEQ ID NO: 274), gene coding GT3 (nucleotides 12915-13757 of the complementary strand of SEQ ID NO: 274), wzy (nucleotides 10707-11846 of the complementary strand of SEQ ID NO: 274), wzx (nucleotides 15037-16476 of the complementary strand of SEQ ID NO: 274), gene coding nucleotide sugar dehydrogenase protein (nucleotides 7234-7680 of the complementary strand of SEQ ID NO: 274) and gene coding acetyltransferase protein (nucleotides 10163-10717 of the complementary strand of SEQ ID NO: 274).
Conserved part: epsR (nucleotides 1-318 of SEQ ID NO: 274), epsB (nucleotides 2220-3005 of SEQ ID NO: 274), epsD (nucleotides 1470-2165 of SEQ ID NO: 274), epsL (nucleotides 6310-7224 of the complementary strand of SEQ ID NO: 274), gene coding LytR protein (nucleotides 5383-6285 of SEQ ID NO: 274), epsC (nucleotides 681-1460 of SEQ ID NO: 274), epsE1 (nucleotides 2992-3591 of SEQ ID NO: 274) and epsE2 (nucleotides 3592-4347 of SEQ ID NO: 274.

The term “exopolysaccharide (EPS)” is well known and the skilled person can routinely determine if a lactic acid bacterium of interest produces EPS. As known and understood by the skilled person a lactic acid bacterium of interest, which produces EPS, will comprise an active eps gene cluster.

As known to the skilled person, as described above, an active eps gene cluster comprises genes involved in regulation and modulation of EPS biosynthesis and genes involved in the biosynthesis of an oligosaccharide repeat unit and export, including a glycosyltransferase (GT), a polymerase and a transporter. In short and as understood by the skilled person, since the lactic acid bacterium strains of the first aspect are capable of producing and exporting exopolysaccharide (EPS), then they will comprise an active eps gene cluster. Zeidan et al., 2017 reviews the production of EPS by LAB, and provide details of the structure of eps gene clusters in LAB.

Preferably, the Lactococcus lactic lactic acid bacterium (LAB) strain comprising an active eps gene cluster capable of producing exopolysaccharide (EPS), wherein the eps gene cluster comprises at least one, preferably two, more preferably three nucleotide sequence selected from the nucleotide sequences (a), (b) and (c), even more preferably all of the nucleotide sequences (a) to (d), as defined in (i), and preferably at least one, preferably all of the nucleotide sequences selected from the nucleotide sequences (1)-(8) as defined in (i) is the strain DSM 33134, or a mutant or a variant thereof.

Preferably, the Lactococcus lactis lactic acid bacterium (LAB) strain comprising an active eps gene cluster capable of producing exopolysaccharide (EPS), wherein the eps gene cluster comprises at least one, preferably two, more preferably three nucleotide sequence selected from the nucleotide sequences (a) to (c5), even more preferably all of the nucleotide sequences (a) to (i), as defined in (ii), and preferably at least one, preferably all of the nucleotide sequences selected from the nucleotide sequences (1)-(8) as defined in (ii) is the strain DSM 33135, or a mutant or a variant thereof.

Preferably, the Lactococcus lactis lactic acid bacterium (LAB) strain comprising an active eps gene cluster capable of producing exopolysaccharide (EPS), wherein the eps gene cluster comprises at least one, preferably two, more preferably three nucleotide sequence selected from the nucleotide sequences (a) to (d), preferably all of the nucleotide sequences (a) to (c3), and even more preferably all of the nucleotide sequences (a) to (d), as defined in (iii), and preferably at least one, preferably all of the nucleotide sequences selected from the nucleotide sequences (1)-(8) as defined in (iii) is the strain DSM 33136 or a mutant or a variant thereof.

Preferably, the Lactococcus lactis lactic acid bacterium (LAB) strain comprising an active eps gene cluster capable of producing exopolysaccharide (EPS), wherein the eps gene cluster comprises at least one, preferably two, more preferably three nucleotide sequence selected from the nucleotide sequences (a)-(d) preferably all of the nucleotide sequences (a) to (c4), even more preferably all of the nucleotide sequences (a) to (d), as defined in (iv), and preferably at least one, preferably all of the nucleotide sequences selected from the nucleotide sequences (1)-(8) as defined in (iv) is the strain DSM 33137 or a mutant or a variant thereof.

Preferably, the Lactococcus lactis lactic acid bacterium (LAB) strain comprising an active eps gene cluster capable of producing exopolysaccharide (EPS), wherein the eps gene cluster comprises at least one, preferably two, more preferably three nucleotide sequence selected from the nucleotide sequences (a) to (c4), preferably all of the nucleotide sequences (a) to (j), as defined in (v), and preferably at least one, preferably all of the nucleotide sequences selected from the nucleotide sequences (1)-(7) as defined in (v) is the strain DSM 33138 or a mutant or a variant thereof.

Preferably, the Lactococcus lactis lactic acid bacterium (LAB) strain comprising an active eps gene cluster capable of producing exopolysaccharide (EPS), wherein the eps gene cluster comprises at least one, preferably two, more preferably three nucleotide sequence selected from the nucleotide sequences (a)-(m), preferably all of the nucleotide sequences (a) to (c6), even more preferably all of the nucleotide sequences (a) to (m), as defined in (vi), and preferably at least one, preferably all of the nucleotide sequences selected from the nucleotide sequences (1)-(8) as defined in (vi) is the strain DSM 33139 or a mutant or a variant thereof.

Preferably, the Lactococcus lactis lactic acid bacterium (LAB) strain comprising an active eps gene cluster capable of producing exopolysaccharide (EPS), wherein the eps gene cluster comprises at least one, preferably two, more preferably three nucleotide sequence selected from the nucleotide sequences (a) to (c2), even more preferably all of the nucleotide sequences (a) to (d), as defined in (vii), and preferably at least one, preferably all of the nucleotide sequences selected from the nucleotide sequences (1)-(8) as defined in (vii) is the strain DSM 33140 or a mutant or a variant thereof.

Preferably, the Lactococcus lactis lactic acid bacterium (LAB) strain comprising an active eps gene cluster capable of producing exopolysaccharide (EPS), wherein the eps gene cluster comprises at least one, preferably two, more preferably three nucleotide sequence selected from the nucleotide sequences (a)-(f), preferably all of the nucleotide sequences (a) to (c6), even more preferably all of the nucleotide sequences (a) to (f), as defined in (viii), and preferably at least one, preferably all of the nucleotide sequences selected from the nucleotide sequences (1)-(9) as defined in (viii) is the strain DSM 33141 or a mutant or a variant thereof.

Preferably, the Lactococcus lactis lactic acid bacterium (LAB) strain comprising an active eps gene cluster capable of producing exopolysaccharide (EPS), wherein the eps gene cluster comprises at least one, preferably two, more preferably three nucleotide sequence selected from the nucleotide sequences (a) to (e), more preferably all of the nucleotide sequences (a) to (c3), even more preferably all of the nucleotide sequences (a) to (e), as defined in (x), and preferably at least one, preferably all of the nucleotide sequences selected from the nucleotide sequences (1)-(9) as defined in (ix) is the strain DSM 33142 or a mutant or a variant thereof.

Preferably, the Lactococcus lactis lactic acid bacterium (LAB) strain comprising an active eps gene cluster capable of producing exopolysaccharide (EPS), wherein the eps gene cluster comprises at least one, preferably two, more preferably three nucleotide sequence selected from the nucleotide sequences (a) to (e), even more preferably all of the nucleotide sequences (a) to (c3), even more preferably all of the nucleotide sequences (a) to (e), as defined in (xi), and preferably at least one, preferably all of the nucleotide sequences selected from the nucleotide sequences (1)-(8) as defined in (x) is the strain DSM 33183 or a mutant or a variant thereof.

As discussed in working examples herein (see, e.g., Table 1)—the herein disclosed novel Lactococcus lactis strains have excellent texturing properties. In addition, as shown in Example 2 and Tables 2 and 3, the herein disclosed novel Lactococcus lactis strains also have excellent texturing properties in plant-based milk, in particular in soy milk supplemented with glucose, such as 0.5-5% glucose, preferably 0.5-2% glucose, more preferably about 2% glucose.

Preferably, the texturing lactic acid bacterium strains (i) to (x) as described herein is a LAB strain which generates fermented milks having a shear stress greater than 40 Pa, such as about 41 Pa, 42 Pa, 43 Pa, 44 Pa, 45 Pa, 46 Pa or more, preferably the LAB strain generates fermented milk having a shear stress of 41 Pa or more, such as about 41 Pa, 48 Pa, 52 Pa, 53 Pa, 55 Pa, 56 Pa, 60 Pa, 64 Pa, 65 Pa or 67 Pa, preferably in the presence of co-acidifier or helper strain, which is preferably selected from DSM 25485, DSM 33192 and/or DSM 33133, even more preferably in the presence of DSM 25485, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

Without being limited to theory, not all of the strains are able to acidify milk in 15 h or less, i.e., to reach a target pH—e.g. pH 4.55 in 15 h or less, measured as follows: 200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until the target pH is reached. Target pH may be e.g. pH between 4 and 5, preferably between pH 4.3 to 4.7, more preferably between pH 4.4 to 4.6, and even more preferably pH 4.45, pH 4.50, or pH 4.55. The inoculation temperature is 30° C.

These strains may be referred to as “slow-acidifying” strains”. For instance, as shown in Table 1, the following strains may be considered to be “slow-acidifying” strains: DSM 33134, 33135, DSM 33136, 33138, 33139, 33141 and 33183.

For the production of fermented milks, it is currently preferred that milk fermentation (acidification) occurs as fast as possible, e.g., in order to avoid the growth of any potential contaminant microorganism. Accordingly, it is preferred that the slow-acidifying strains are used in combination with a further lactic acid bacterium strain, which, in the context of the present invention, is referred to as “co-acidifier” or “helper” strain. The co-acidifier or helper strain would help the “slow-acidifying” strains to acidify milk in lower amount of time. Without being limited to theory, it is currently believed that the co-acidifier or helper strain would inter alia metabolize the proteins present in milk (casein) faster than the “slow-acidifying” strain, so that the “slow-acidifying” strain would have more available nitrogen source for their growth, which would then be facilitated. LAB require an exogenous source of amino acids or peptides, which are provided by the proteolysis of milk proteins, e.g., casein, which the most abundant protein in milk and the main source of amino acids (Savijoki, K., et al., Appl Microbiol Biotechnol (2006) 71: 394-406).

Slow-acidifying strains are often associated with low proteolytic activity. Proteolysis is the breakdown of proteins into smaller polypeptides or amino acids. A cell wall proteinase (Prt) hydrolyses milk proteins, such as casein, providing a nitrogen source, which makes milk suitable for rapid growth of strains. Other factors than the prt activity, such as carbon metabolism, ldh and codY activities can also play a role. It is not enough to have high prt activity to acidify milk fast. Uptake and further degradation of peptides are also important for the milk acidification rate. Moreover, EPS production is a highly energy demanding process (Zeidan et al., 2017). Texturing L. lactis strains are generally slower in acidifying milk than the non-texturing strains (Poulsen et al., 2019).

Strains which are able to acidify milk in about 15 h or less may be referred to as “fast-acidifying” strains, i.e., strains which are able to reach target pH in 15 h or less, measured as follows: 200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until the target pH is reached. The inoculation temperature is 30° C. Target pH may be e.g. pH between 4 and 5, preferably between pH 4.3 to 4.7, more preferably between pH 4.4 to 4.6, and even more preferably pH 4.45, pH 4.50, or pH 4.55.

These strains may be used on their own or in combination with other strains for the generation of fermented milks. For instance, the following strains may be considered as “fast-acidifying” strains: DSM 33137, 33140 and 33142.

The co-acidifier or helper strain according with the present invention may be any lactic acid bacterium strain which is able to:
i) generate fermented milks with a pH of about 4.55 in 15 h or less, preferably in 12 h or less, measured under the following conditions:
200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until a pH of about 4.55 is reached. Therefore, the “time-to-pH 4.55” can be calculated for a certain lactic acid bacterium strain; and
ii) generate fermented milks having a shear stress of 40 Pa or more measured at shear rate 300 s⁻¹, measured under following conditions:
200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, time to pH 4.55) followed by storage at 4° C. for 5 days, followed by gently stirring and measuring the shear stress at shear rate 300 s⁻¹, wherein the inoculation temperature is 30° C.

In a preferred embodiment, the co-acidifier or helper strain is a lactic acid bacterium strain Lactococcus lactic comprising an active eps gene cluster capable of producing exopolysaccharide (EPS), wherein the eps gene cluster comprises the nucleotide sequences (a), (b) and (c) ((a) to (c4)) as defined in (xi):
(xi) (a): a nucleotide sequence encoding a polypeptide having polymerase activity and having at least 95% identity with the amino acid sequence encoded by nucleotides 6955-8145 of SEQ ID NO: 183 (herein termed wzy);
   (b): a nucleotide sequence encoding a polypeptide having polysaccharide transporter activity and having at least 95% identity with the amino acid sequence encoded by nucleotides 9309-10727 of SEQ ID NO: 183 (herein termed wzx); and
   (c): a nucleotide sequence encoding a polypeptide having glycosyltransferase (GT) activity comprising:
   (c1): a nucleotide sequence having at least 95% identity with the amino acid sequence encoded by nucleotides 4008-4478 of SEQ ID NO: 183 (herein termed GT1);
   (c2): a nucleotide sequence having at least 95% identity with the amino acid sequence encoded by nucleotides 4478-4960 of SEQ ID NO: 183 (herein termed GT2);
   (c3): a nucleotide sequence having at least 95% identity with the amino acid sequence encoded by nucleotides 5015-5965 of SEQ ID NO: 183 (herein termed GT3); and
   (c4): a nucleotide sequence having at least 95% identity with the amino acid sequence encoded by nucleotides 6026-6955 of SEQ ID NO: 183 (herein termed GT4).

In a further preferred embodiment, a lactic acid bacterium strain Lactococcus lactis comprising an active eps gene cluster capable of producing exopolysaccharide (EPS), wherein the eps gene cluster is as defined in (xii):
(xii) SEQ ID NO.: 290.

The skilled person would be able to find further co-acidifier or helper strains suitable for the present invention. For instance, a suitable co-acidifier or helper strain may be a lactic acid bacterium strain Lactococcus lactis comprising an active eps gene cluster capable of producing exopolysaccharide (EPS), wherein the eps gene cluster is as defined in SEQ ID NO.: 291-294 wherein the co-acidifier or helper strain is able to (i) generate fermented milks with a pH of about 4.55 in about 15 h or less, preferably in about 12 h or less, measured as described above, and is able to (ii) generate fermented milks having a shear stress of 40 Pa or more measured at shear rate 300 s⁻¹, measured as described above.

For instance, the following strains may also be used as co-acidifier or helper strains in the context of the present invention: strains DSM 33193, DSM 33133, DSM 33196, DSM 33197, DSM 33200, DSM 33201, DSM 33203, DSM 33204, DSM 33205, DSM 33218, DSM 33219, DSM 33220, DSM 33221, DSM 33222, DSM 33224, DSM 33225, DSM 33140, DSM 33142 and/or DSM 33137, preferably strains DSM 33193, DSM 33196, DSM 33197, DSM 33200, DSM 33201, DSM 33205, DSM 33218, DSM 33220, DSM 33221, DSM 33222, DSM 33224, DSM 33225, and/or DSM 33137.

Accordingly, the present invention further provides the use of any one of strains DSM 33193, DSM 33133, DSM 33196, DSM 33197, DSM 33200, DSM 33201, DSM 33203, DSM 33204, DSM 33205, DSM 33218, DSM 33219, DSM 33220, DSM 33221, DSM 33222, DSM 33224, DSM 33225, DSM 33140, DSM 33142 and/or DSM 33137, preferably the use of any one of strains DSM 33193, DSM 33196, DSM 33197, DSM 33200, DSM 33201, DSM 33205, DSM 33218, DSM 33220, DSM 33221, DSM 33222, DSM 33224, DSM 33225, and/or DSM 33137 as a co-acidifier or helper strain.

In addition, the texturing lactic acid bacterium strains (i) to (x) as described herein is a LAB strain which generates fermented milks having a shear stress greater than 24 Pa, such as about 27 Pa, 28 Pa, 29 Pa, 30 Pa, 32 Pa, 35 Pa, 37 Pa, 42 Pa, 47 Pa, 54 Pa, 59 Pa or more, measured at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH^{˜}4.55 (or higher, if a strain stops acidifying at a higher pH, se Table 3, such as pH 4.55, 4.48, 4.71, 4.64, 4.68, 4.58, 4.4, 4.56, 4.58 or 4.86), followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

For example, the texturing lactic acid bacterium strain (i), which is preferably Lactococcus lactis strain DSM 33134, or a mutant or a variant therefrom, generates fermented milks having a shear stress greater than 40 Pa, preferably greater than 45 Pa, such as about 46 Pa, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

The texturing lactic acid bacterium strain (i), which is preferably Lactococcus lactis strain DSM 33134, or a mutant or a variant therefrom, generates fermented milks having a shear stress greater than 45 Pa, preferably greater than 50 Pa, such as about 53 Pa, in the presence of the co-acidifier strain DSM 25485, preferably in a ratio of about 9:1 (LAB of (i): LAB strain DSM 25485), at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

For example, the texturing lactic acid bacterium strain (i), which is preferably Lactococcus lactis strain DSM 33134, or a mutant or a variant therefrom, generates fermented milks having a shear stress greater than 24 Pa, preferably greater than 30 Pa, such as about 37 Pa, measured at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH 4.56, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

For example, the texturing lactic acid bacterium strain (ii), which is preferably Lactococcus lactis strain DSM 33135, or a mutant or a variant therefrom, generates fermented milks having a shear stress greater than 40 Pa, such as about 43 Pa, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

The texturing lactic acid bacterium strain (ii), which is preferably Lactococcus lactis strain DSM 33135, or a mutant or a variant therefrom, generates fermented milks having a shear stress greater than 45 Pa, preferably greater than 50 Pa, more preferably greater than 60 Pa, such as about 65 Pa, in the presence of the co-acidifier strain DSM 25485, preferably in a ratio of about 9:1 (LAB of (ii):LAB strain DSM 25485), at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

For example, the texturing lactic acid bacterium strain (ii), which is preferably Lactococcus lactis strain DSM 33135, or a mutant or a variant therefrom, generates fermented milks having a shear stress greater than 24 Pa, such as about 30 Pa, measured at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH 4.68, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

For example, the texturing lactic acid bacterium strain (iii), which is preferably Lactococcus lactis strain DSM 33136, or a mutant or a variant therefrom, generates fermented milks having a shear stress greater than 40 Pa, such as about 41 Pa, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

The texturing lactic acid bacterium strain (iii), which is preferably Lactococcus lactis strain DSM 33136, or a mutant or a variant therefrom, generates fermented milks having a shear stress greater than 45 Pa, preferably greater than 50 Pa, more preferably greater than 55 Pa, such as about 60 Pa, in the presence of the co-acidifier strain DSM 25485, preferably in a ratio of about 9:1 (LAB of (iii):LAB strain DSM 25485), at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

For example, the texturing lactic acid bacterium strain (iii), which is preferably Lactococcus lactis strain DSM 33136, or a mutant or a variant therefrom, generates fermented milks having a shear stress greater than 24 Pa, such as about 21 Pa, measured at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH 4.64, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

For example, the texturing lactic acid bacterium strain (iv), which is preferably Lactococcus lactis strain DSM 33137, or a mutant or a variant therefrom, generates fermented milks having a shear stress greater than 40 Pa, such as about 45 Pa, preferably greater than 45 Pa, such as about 48 Pa, measured at shear rate 300 s⁻¹, under the following conditions: 200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

The texturing lactic acid bacterium strain (iv), which is preferably Lactococcus strain DSM 33137, or a mutant or a variant therefrom, generates fermented milks having a shear stress greater than 45 Pa, such as about 48 Pa, in the presence of the co-acidifier strain DSM 25485, preferably in a ratio of about 9:1 (LAB of (iv):LAB strain DSM 25485), at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

For example, the texturing lactic acid bacterium strain (iv), which is preferably Lactococcus lactis strain DSM 33137, or a mutant or a variant therefrom, generates fermented milks having a shear stress greater than 24 Pa, such as about 30 Pa, preferably greater than 30 Pa, such as about 35 Pa, measured at shear rate 300 s⁻¹, under the following conditions: 200 ml of soy milk supplemented with 2% glucose (as described in Example 2) are inoculated with 2 ml of an overnight culture of the lactic acid bacterium strain, and left at inoculation temperature until pH 4.40, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

For example, the texturing lactic acid bacterium strain (v), which is preferably Lactococcus lactis strain DSM 33138, or a mutant or a variant therefrom, generates fermented milks having a shear stress greater than 45 Pa, preferably greater than 50 Pa, such as about 55 Pa, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

The texturing lactic acid bacterium strain (v), which is preferably Lactococcus lactis strain DSM 33138, or a mutant or a variant therefrom, generates fermented milks having a shear stress greater than 45 Pa, preferably greater than 50 Pa, more preferably greater than 60 Pa, even more preferably greater than 65 Pa, such as about 67 Pa, in the presence of the co-acidifier strain DSM 25485, preferably in a ratio of about 9:1 (LAB of (v):LAB strain DSM 25485), at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

For example, the texturing lactic acid bacterium strain (v), which is preferably Lactococcus lactis strain DSM 33138, or a mutant or a variant therefrom, generates fermented milks having a shear stress greater than 24 Pa, such as about 27 Pa, measured at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH 4.71, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

For example, the texturing lactic acid bacterium strain (vi), which is preferably Lactococcus lactis strain DSM 33139, or a mutant or a variant therefrom, generates fermented milks having a shear stress greater than 40 Pa, preferably greater than 45 Pa, more preferably greater 50 Pa, such as about 52 Pa, in the presence of the co-acidifier strain DSM 25485, preferably in a ratio of about 9:1 (LAB of (vi):LAB strain DSM 25485), at shear rate 300 s⁻¹, under the following conditions: 200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

For example, the texturing lactic acid bacterium strain (vi), which is preferably Lactococcus lactis strain DSM 33139, or a mutant or a variant therefrom, generates fermented milks having a shear stress greater than 24 Pa, preferably greater than 30 Pa, more preferably greater 35 Pa, such as about 42 Pa, at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH 4.58, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

For example, the texturing lactic acid bacterium strain (vii), which is preferably Lactococcus lactis strain DSM 33140, or a mutant or a variant therefrom, generates fermented milks having a shear stress greater than 40 Pa, preferably greater than 45 Pa, such as about 46 Pa, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

The texturing lactic acid bacterium strain (vii), which is preferably Lactococcus lactis strain DSM 33140, or a mutant or a variant therefrom, generates fermented milks having a shear stress greater than 45 Pa, preferably greater than 50 Pa, more preferably greater than 55 Pa, even more preferably greater than 60 Pa, such as about 64 Pa, in the presence of the co-acidifier strain DSM 25485, preferably in a ratio of about 9:1 (LAB of (vii):LAB strain DSM 25485), at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

For example, the texturing lactic acid bacterium strain (vii), which is preferably Lactococcus lactis strain DSM 33140, or a mutant or a variant therefrom, generates fermented milks having a shear stress greater than 24 Pa, such as about 28 Pa, measured at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

For example, the texturing lactic acid bacterium strain (viii), which is preferably Lactococcus lactis strain DSM 33141, or a mutant or a variant therefrom, texturing generates fermented milks having a shear stress greater than 40 Pa, such as about 41 Pa, in the presence of the co-acidifier strain DSM 25485, preferably in a ratio of about 9:1 (LAB of (viii):LAB strain DSM 25485), at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

For example, the texturing lactic acid bacterium strain (viii), which is preferably Lactococcus lactis strain DSM 33141, or a mutant or a variant therefrom, texturing generates fermented milks having a shear stress greater than 24 Pa, preferably greater than 30 Pa, such as about 32 Pa, at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH 4.58, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

For example, the texturing lactic acid bacterium strain (ix), which is preferably Lactococcus lactis strain DSM 33142, or a mutant or a variant therefrom, generates fermented milks having a shear stress greater than 45 Pa, preferably greater than 50 Pa, more preferably greater than 55 Pa, such as about 56 Pa, at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

For example, the texturing lactic acid bacterium strain (ix), which is preferably Lactococcus lactis strain DSM 33142, or a mutant or a variant therefrom, generates fermented milks having a shear stress greater than 24 Pa, preferably greater than 30 Pa, more preferably greater than 40 Pa, such as about 42 Pa, at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

For example, the texturing lactic acid bacterium strain (x), which is preferably Lactococcus lactis strain DSM 33183, or a mutant or a variant therefrom, generates fermented milks having a shear stress greater than 60 Pa, preferably greater than 65 Pa, more preferably greater than 70 Pa, such as about 72 Pa, at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

For example, the texturing lactic acid bacterium strain (x), which is preferably Lactococcus lactis strain DSM 33183, or a mutant or a variant therefrom, generates fermented milks having a shear stress greater than 24 Pa, preferably greater than 30 Pa, more preferably greater than 50 Pa, such as about 59 Pa, at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH 4.86, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

The present invention also provides a Lactococcus lactis strain selected from the following strains:
(i)—Lactococcus lactis strain DSM 33134 and strains derived from DSM 33134, wherein the derived strain is characterized as having at least the same texturing capability as DSM 33134;
(ii)—Lactococcus lactis strain DSM 33135 and strains derived from DSM 33135, wherein the derived strain is characterized as having at least the same texturing capability as DSM 33135;
(iii)—Lactococcus lactis strain DSM 33136 and strains derived from DSM 33136, wherein the derived strain is characterized as having at least the same texturing capability as DSM 33136;
(iv)—Lactococcus lactis strain DSM 33137 and strains derived from DSM 33137, wherein the derived strain is characterized as having at least the same texturing capability as DSM 33137;
(v)—Lactococcus lactis strain DSM 33138 and strains derived from DSM 33138, wherein the derived strain is characterized as having at least the same texturing capability as DSM 33138;
(vi)—Lactococcus lactis strain DSM 33139 and strains derived from DSM 33139, wherein the derived strain is characterized as having at least the same texturing capability as DSM 33139;
(vii)—Lactococcus lactis strain DSM 33140 and strains derived from DSM 33140, wherein the derived strain is characterized as having at least the same texturing capability as DSM 33140;
(viii)—Lactococcus lactis DSM 33141 and strains derived from DSM 33141, wherein the derived strain is characterized as having at least the same texturing capability as DSM 33141;
(ix)—Lactococcus lactis strain DSM 33142 and strains derived from DSM 33142, wherein the derived strain is characterized as having at least the same texturing capability as DSM 33142;
(x)—Lactococcus lactis strain DSM 33183 and strains derived from DSM 33183, wherein the derived strain is characterized as having at least the same texturing capability as DSM 33183.

In addition, the present invention provides the following Lactococcus lactic strain, DSM 33192 and strains derived from DSM 33192, wherein the derived strain is characterized as having at least the same texturing capability as DSM 33192. The eps gene cluster of DSM 33192 comprises the following genes in the variable and conserved parts:
Variable part: wzy (SEQ ID NO.: 194), wzx (SEQ ID NO.: 196), gene coding GT1 (SEQ ID NO.: 190), gene coding GT2 (SEQ ID NO.: 191), gene coding GT3 (SEQ ID NO.: 192), gene coding GT4 (SEQ ID NO.: 193) and gene coding a glycerophosphotransferase family protein (SEQ ID NO.: 195).
Conserved part: epsR (SEQ ID NO.: 184), epsX (SEQ ID NO.: 185), epsB (SEQ ID NO.: 188), epsD (SEQ ID NO.: 187), epsL (SEQ ID NO.: 197), gene coding LytR protein (SEQ ID NO.: 198), epsC (SEQ ID NO.: 186) and epsE (SEQ ID NO.: 189).

Strain DSM 33192, or a mutant or a variant therefrom, generates fermented milks having a shear stress greater than 70 Pa, preferably greater than 80 Pa, more preferably greater than 85 Pa, even more preferably greater than 90 Pa, such as about 94 Pa, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 5.6, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

Strain DSM 33192, or a mutant or a variant therefrom, generates fermented milks having a shear stress greater than 24 Pa, preferably greater than 30 Pa, more preferably greater than 40 Pa, even more preferably greater than 45 Pa, such as about 47 Pa, measured at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

The Composition Comprising a LAB of the Invention

In a second aspect, the present invention provides a composition comprising one or more of the texturing Lactococcus lactis strains of the invention as described in the first aspect of the present invention.

Accordingly, the composition of the invention comprises at least one of the LAB (i) to (x) as described above in the context of the first aspect of the present invention.

In particular, the present invention provides a composition comprising one or more of the texturing Lactococcus lactis strains of the invention as described in the first aspect of the present invention and a co-acidifier or helper strain as defined in the first aspect of the present invention. In a preferred embodiment, the composition of the present invention comprises one or more of the texturing Lactococcus lactis strains of the invention as described in the first aspect of the present invention and a co-acidifier or helper strain as defined in the first aspect of the present invention in a ratio of about 9:1 (LAB strain(s) of the present invention: co-acidifier or helper strain(s)).

Preferably, the composition of the present invention comprises at least one Lactococcus lactis lactic acid bacterium strain according to the first aspect of the present invention and one or more further lactic acid bacterium strain(s), wherein the one or more further lactic acid bacterium strain(s) is(are) able to:

i) generate fermented milks with a pH of about 4.55 in about 15 h or less, preferably in about 12 h or less, measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until a pH of about 4.55 is reached. Therefore, the “time-to-pH 455” can be calculated for a certain lactic acid bacterium strain; and

ii) generate fermented milks having a shear stress of 40 Pa or more measured at shear rate 300 s⁻¹, measured under following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, time to pH 4.55) followed by storage at 4° C. until shear stress is measured, typically from 1-7 days, such as for 5 days, followed by gently stirring and measuring the shear stress at shear rate 300 s⁻¹, wherein the inoculation temperature is 30° C.

More preferably, the composition of the present invention comprises at least one Lactococcus lactis lactic acid bacterium strain (i) to (x) according to the first aspect of the present invention in combination with (a) at least one lactic acid bacterium strain Lactococcus lactis comprising an active eps gene cluster capable of producing exopolysaccharide (EPS), wherein the eps gene cluster comprises the nucleotide sequences (a), (b) and (c) (a to c4) as defined in (xi), or (b) a lactic acid bacterium strain Lactococcus lactis comprising an active eps gene cluster capable of producing exopolysaccharide (EPS), wherein the eps gene cluster is as defined in (xii):
(xi) (a): a nucleotide sequence encoding a polypeptide having polymerase activity and having at least 70%, preferably at least 85%, more preferably at least 95%, even more preferably at least 98%, and most preferably 100% identity with the amino acid sequence encoded by nucleotides 6955-8145 of SEQ ID NO:183 (herein termed wzy);
   (b): a nucleotide sequence encoding a polypeptide having polysaccharide transporter activity and having at least 70%, preferably at least 85%, more preferably at least 95%, even more preferably at least 98%, and most preferably 100% identity with the amino acid sequence encoded by nucleotides 9309-10727 of SEQ ID NO:183 (herein termed wzx); and
   (c): a nucleotide sequence encoding a polypeptide having glycosyltransferase (GT) activity comprising:
   (c1): a nucleotide sequence having at least 70%, preferably at least 85%, more preferably at least 95%, even more preferably at least 98%, and most preferably 100% identity with the amino acid sequence encoded by nucleotides 4008-4478 of SEQ ID NO:183 (herein termed GT1);
   (c2): a nucleotide sequence having at least 70%, preferably at least 85%, more preferably at least 95%, even more preferably at least 98%, and most preferably 100% identity with the amino acid sequence encoded by nucleotides 4478-4960 of SEQ ID NO:183 (herein termed GT2);
   (c3): a nucleotide sequence having at least 70%, preferably at least 85%, more preferably at least 95%, even more preferably at least 98%, and most preferably 100% identity with the amino acid sequence encoded by nucleotides 5015-5965 of SEQ ID NO:183 (herein termed GT3); and
   (c4): a nucleotide sequence having at least 70%, preferably at least 85%, more preferably at least 95%, even more preferably at least 98%, and most preferably 100% identity with the amino acid sequence encoded by nucleotides 6026-6955 of SEQ ID NO:183 (herein termed GT4);
(xii) SEQ ID NO.: 290.

In a further preferred embodiment, the composition of the present invention comprises at least one Lactococcus lactis lactic acid bacterium strain according to the first aspect of the present invention and one or more lactic acid bacterium strains comprising an active eps gene cluster capable of producing exopolysaccharide (EPS), wherein the eps gene cluster is as defined in SEQ ID NO.: 291-294 wherein the further strain is able to (i) generate fermented milks with a pH of about 4.55 in about 15 h or less, preferably in about 12 h or less, measured as defined above, and is able to (ii) generate fermented milks having a shear stress of 40 Pa or more measured at shear rate 300 s⁻¹, measured as described above.

In a further preferred embodiment, the composition of the present invention comprises at least one Lactococcus lactis lactic acid bacterium strain (i) to (x), according to the first aspect of the present invention, and one or more lactic acid bacterium strains selected from strains DSM 33193, DSM 33133, DSM 33196, DSM 33197, DSM 33200, DSM 33201, DSM 33203, DSM 33204, DSM 33205, DSM 33218, DSM 33219, DSM 33220, DSM 33221, DSM 33222, DSM 33224, DSM 33225, DSM 33140, DSM 33142, DSM 33137, DSM 33192 and/or DSM 25485, preferably selected from strains DSM 33193, DSM 33196, DSM 33197, DSM 33200, DSM 33201, DSM 33205, DSM 33218, DSM 33220, DSM 33221, DSM 33222, DSM 33224, DSM 33225, DSM 33137, DSM 33192 and/or DSM 25485.

More preferably, the composition of the invention comprises at least one, preferably one, of the LAB (i) to (x) as described above in the context of the first aspect of the present invention and
(i) the LAB strain Lactococcus lactis subsp. cremoris DSM 25485, or a mutant or variant therefrom; and/or
(ii) the lactic acid bacterium strain Lactococcus lactis subsp. lactis DSM 33192, or a mutant or variant therefrom; and/or
(iii) the lactic acid bacterium strain Lactococcus lactis DSM 33133, or a mutant or variant therefrom.

For example, the composition of the present invention comprises strain DSM 33134 and strain DSM 25485. For example, the composition of the present invention comprises strain DSM 33135 and strain DSM 25485. For example, the composition of the present invention comprises strain DSM 33136 and strain DSM 25485. For example, the composition of the present invention comprises strain DSM 33137 and strain DSM 25485. For example, the composition of the present invention comprises strain DSM 33138 and strain DSM 25485. For example, the composition of the present invention comprises strain DSM 33139 and strain DSM 25485. For example, the composition of the present invention comprises strain DSM 33140 and strain DSM 25485. For example, the composition of the present invention comprises strain DSM 33141 and strain DSM 25485.

For example, the composition of the present invention may comprise one or more of strains DSM 33134, DSM 33135, DSM 33136, DSM 33137, DSM 33138, DSM 33139, DSM 33140, DSM 33142 and/or DSM 33141 and one or more of the co-acidifier or helper strains as defined in the first aspect of the present invention, preferably one or more of the following strains: DSM 25485, DSM 33192 and/or DSM 33133.

In a further embodiment, the composition of the present invention may comprise strain DSM 33134 and strain DSM 24649. In a further embodiment, the composition of the present invention may comprise strain DSM 33139 and strain DSM 24649.

Preferably, the composition of the present invention in any of its embodiments comprises at least 1×10⁶ CFU (colony-forming units)/ml total LAB strains. It may be preferred that the composition comprises at least 1×10⁸ CFU/ml of the at least one, preferably one, lactic acid bacterium strain according to the invention.

In a further embodiment, the composition of the invention may also comprise at least one Lactococcus lactis lactic acid bacterium strain according to the first aspect of the present invention and yeast extract, preferably yeast extract in an amount of 0.2%. Yeast extract can be obtained from any source available to the skilled person, such as from Procelys (e.g., Yeast Extract NuCel® 545 MG, batch number 0005115910, batch AD 18 A05030). For example, the composition of the present invention may comprise one or more of strains DSM 33134, DSM 33137, DSM 33139 and/or DSM 33140 and yeast extract, preferably in an amount of 0.2%, as described above. As it can be seen in Table 1, the presence of yeast may decrease the time-to-pH 4.55, measured as described above, and may lead to fermented milks with higher shear stress.

As described above in the context of the first aspect of the present invention, the LAB of the present invention, either alone or in combination with co-acidifier or helper strain, preferably LAB strain Lactococcus lactis strain DSM 25485, are able to generate fermented milks having high shear stress. Accordingly, the composition of the present invention is able to generate at least the same shear stress as the one described for the LAB of the present invention, either alone or in the presence of a co-acidifier or helper strain, as described in the context of the first aspect of the present invention.

Lactic acid bacteria, including bacteria of the species Lactococcus sp., are normally supplied to the dairy industry either as frozen (F-DVS) or freeze-dried (FD-DVS) cultures for bulk starter propagation or as so-called “Direct Vat Set” (DVS) cultures, intended for direct inoculation into a fermentation vessel or vat for the production of a dairy product, such as a fermented milk product. Such lactic acid bacterial cultures are in general referred to as “starter cultures” or “starters”. Accordingly, the composition of the present invention may be frozen or freeze-dried. In addition, the composition of the present invention may be provided in liquid form. Thus, in one embodiment, the composition is in frozen, dried, freeze-dried or liquid form.

The composition of the present invention may additionally comprise cryoprotectants, lyoprotectants, antioxidants, nutrients, fillers, flavorants or mixtures thereof. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as croprotectants and lyoprotectants are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate).

In one embodiment, the composition according to the present invention may comprise one or more cryoprotective agent(s) selected from the group consisting of inosine-5′-monophosphate (IMP), adenosine-5′-monophosphate (AMP), guanosine-5′-monophosphate (GMP), uranosine-5′-monophosphate (UMP), cytidine-5′-monophosphate (CMP), adenine, guanine, uracil, cytosine, adenosine, guanosine, uridine, cytidine, hypoxanthine, xanthine, hypoxanthine, orotidine, thymidine, inosine and a derivative of any such compounds. Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

In one embodiment of the invention the cryoprotective agent is an agent or mixture of agents, which in addition to its cryoprotectivity has a booster effect.

The expression “booster effect” is used to describe the situation wherein the cryoprotective agent confers an increased metabolic activity (booster effect) on to the thawed or reconstituted culture when it is inoculated into the medium to be fermented or converted. Viability and metabolic activity are not synonymous concepts. Commercial frozen or freeze-dried cultures may retain their viability, although they may have lost a significant portion of their metabolic activity, e.g., cultures may lose their acid-producing (acidification) activity when kept stored even for shorter periods of time. Thus, viability and booster effect have to be evaluated by different assays. Whereas viability is assessed by viability assays such as the determination of colony forming units, booster effect is assessed by quantifying the relevant metabolic activity of the thawed or reconstituted culture relative to the viability of the culture. The term “metabolic activity” refers to the oxygen removal activity of the cultures, its acid-producing activity, i. e. the production of, e. g., lactic acid, acetic acid, formic acid and/or propionic acid, or its metabolite producing activity such as the production of aroma compounds such as acetaldehyde, (a-acetolactate, acetoin, diacetyl and 2,3-butylene glycol (butanediol)).

In one embodiment the composition of the invention contains or comprises from 0.2% to 20% of the cryoprotective agent or mixture of agents measured as % w/w of the material. It is, however, preferable to add the cryoprotective agent or mixture of agents at an amount which is in the range from 0.2% to 15%, from 0.2% to 10%, from 0.5% to 7%, and from 1% to 6% by weight, including within the range from 2% to 5% of the cryoprotective agent or mixture of agents measured as % w/w of the frozen material by weight. In a preferred embodiment the culture comprises approximately 3% of the cryoprotective agent or mixture of agents measured as % w/w of the material by weight. The amount of approximately 3% of the cryoprotective agent corresponds to concentrations in the 100 mM range. It should be recognized that for each aspect of embodiment of the invention the ranges may be increments of the described ranges.

In one embodiment the composition of the invention may comprise thickener and/or stabilizer, such as pectin (e.g. HM pectin, LM pectin), gelatin, CMC, Soya Bean Fiber/Soya Bean Polymer, starch, modified starch, carrageenan, alginate, and guar gum.

In one embodiment wherein the microorganism produces a polysaccharide (such as EPS) which causes a high/ropy texture in the acidified milk product the acidified milk product is produced substantially free, or completely free of any addition of thickener and/or stabilizer, such as pectin (e.g. HM pectin, LM pectin), gelatin, CMC, Soya Bean Fiber/Soya Bean Polymer, starch, modified starch, carrageenan, alginate, and guar gum. By substantially free should be understood that the product comprises from 0% to 20% (w/w) (e.g. from 0% to 10%, from 0% to 5% or from 0% to 2% or from 0% to 1%) thickener and/or stabilizer.

Use of the LAB Strains for Increasing the Viscosity of a Fermented Milk Product

In a third aspect, the present invention provides the use of the LAB or the present invention, as described in the first aspect, and/or the use of the composition of the present invention, as described in the second aspect, for increasing the viscosity of a fermented milk product. Hence, in the third aspect, the present invention provides a method for increasing the viscosity (i.e., for improving the texture) of a fermented milk product, wherein the method comprises the use of the LAB or the present invention, as described in the first aspect, and/or the use of the composition of the present invention, as described in the second aspect.

As described above, the LAB strains (i) to (x) of the present invention, as described in the first aspect, and the compositions of the present invention, as described in the second aspect, are able to generate fermented milks having a shear stress greater than 40 Pa, such as about 41 Pa, 42 Pa, 43 Pa, 44 Pa, 45 Pa, 46 Pa or more, preferably the LAB strains/compositions of the present invention generate fermented milk having a shear stress of 54 Pa or more, such as about 48 Pa, 52 Pa, 53 Pa, 60 Pa, 64 Pa, 65 Pa, 66 Pa, 67 Pa, 70 Pa or 72 Pa, preferably in the presence of co-acidifier or helper strain, which is preferably selected from DSM 25485, DSM 33192, and/or DSM 33133, even more preferably strain DSM 25485, measured at shear rate 300 s⁻¹, under the following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

As described above, the LAB strains (i) to (x) of the present invention, as described in the first aspect, and the compositions of the present invention, as described in the second aspect, are able to generate fermented milks having a shear stress greater than 24 Pa, such as about 27 Pa, 28 Pa, 29 Pa, 30 Pa, 32 Pa, 35 Pa, 37 Pa, 42 Pa, 47 Pa, 54 Pa, 59 Pa or more, measured at shear rate 300 s⁻¹, under the following conditions:

200 ml of soy milk supplemented with 2% glucose (as described in Example 2) are inoculated with 2 ml of an overnight culture of the lactic acid bacterium strain, and left at inoculation temperature until pH^{˜}4.55 (such as pH 4.55, 4.48, 4.71, 4.64, 4.68, 4.58, 4.4, 4.56, 4.58 or 4.86), followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

For the specific shear stress of milk fermented with the specific LAB strains of the invention, either alone or in the presence of an acidifying LAB strain, we refer to the first aspect of the present invention.

As discussed in the context of the first aspect of the present invention, some of the LAB strains of the present invention are able to acidify mammalian milk in 15 h or less, measured as described in the first aspect of the present invention (“fast-acidifying” strains). These strains may thus be preferably used on their own or in combination with other strains for the generation of fermented milks, in particular for their use of fermented milk with increased viscosity.

In addition, there are some LAB strains of the present invention that are not able to acidify mammalian milk in 15 h or less, measured as described in the first aspect of the present invention. They may be referred to as “slow-acidifying” strains (e.g., DSM 33134, 33135, DSM 33136, 33138, 33139, 33141 and/or 33183). These strains may advantageously be used in the presence of a co-acidifier or helper strain as defined in the first aspect of the present invention. In particular, these strains may be advantageously used in the presence of strain DSM 25485, and/or strain DSM 33192, and/or strain DSM 33133. As described above, preferably, the one or more of the texturing Lactococcus lactic strains of the invention as described in the first aspect of the present invention and a co-acidifier or helper strain as defined in the first aspect of the present invention are used in combination in a ratio of about 9:1 (LAB strain(s) of the present invention: co-acidifier or helper strain(s)).

As shown in Table 1, when milk is fermented with one of the LAB strains of the present invention and the co-acidifier strain DSM 25485, the shear stress values of milk are increased and/or the “time-to-pH 4.55” is decreased. Without being limited to theory, it is believed that, as described above, the proteolytic nature of DSM 25485 allows and/or facilitates the growth of the LAB of the present invention. In addition, it is believed that a combination of the EPS produced by DSM 25485 and the EPS produced by the strain of the present invention results in the enhanced viscosity of the fermented milk observed, measured as shear stress, as described above.

Without being limited to theory, it is believed that effect in increased in shear stress of milk fermented with one of the LAB of the present invention and the co-acidifier strain DSM 25485 (see Table 1) would also be obtained when milk is incubated with one of the LAB of the present invention (i) to (x), as described above, and strain DSM 33192. Strain DSM 33192 is also a proteolytic strain and produces EPS with similar structure as the structure of the EPS produced by strain DSM 25485.

In addition, without being limited to theory, it is it is believed that effect in increase in shear stress of milk fermented with one of the LAB of the present invention and the co-acidifier strain DSM 25485 (see Table 1) would also be obtained when milk is incubated with one of the LAB of the present invention (i) to (x), as described above, and one or more of the following strains: DSM 33193, DSM 33133, DSM 33196, DSM 33197, DSM 33200, DSM 33201, DSM 33203, DSM 33204, DSM 33205, DSM 33218, DSM 33219, DSM 33220, DSM 33221, DSM 33222, DSM 33224, DSM 33225, DSM 33140, DSM 33142, DSM 33137, DSM 33192 and/or DSM 25485, preferably one or more of the following strains DSM 33193, DSM 33196, DSM 33197, DSM 33200, DSM 33201, DSM 33205, DSM 33218, DSM 33220, DSM 33221, DSM 33222, DSM 33224, DSM 33225, DSM 33137, DSM 33192 and/or DSM 25485. These strains are able to:

i) generate fermented milks with a pH of about 4.55 in about 15 h or less, preferably in about 12 h or less, measured under the following conditions:

200 ml semi-fat milk (1.5% fat) is heated to 90° C. for 20 min, followed by cooling to inoculation temperature (30° C.), and inoculated with 2 ml of an overnight culture of the lactic acid bacterium strain, and left at inoculation temperature until a pH of about 4.55 is reached. Therefore, the “time-to-pH 455” can be calculated for a certain lactic acid bacterium strain; and

ii) generate fermented milks having a shear stress of 40 Pa or more measured at shear rate 300 s⁻¹, measured under following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55, time to pH 4.55) followed by storage at 4° C. until shear stress is measured, typically from 1-7 days, such as for 5 days, followed by gently stirring and measuring the shear stress at shear rate 300 s⁻¹, wherein the inoculation temperature is 30° C.

In a specific embodiment of the third aspect, the present invention provides the use of the Lactococcus lactic subsp. cremoris strain DSM 25485, for increasing viscosity of a fermented milk product.

The present inventors have found that strain DSM 25485 generated fermented milks having a shear stress greater than 45 Pa, preferably greater than 50 Pa, more preferably greater than 55 Pa, such as 56 Pa, see Table 1, measured at shear rate 300 s⁻¹, measured under following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

In addition, the present inventors have surprisingly found that strain DSM 25485 generated fermented milks having a shear stress greater than 24 Pa, preferably greater than 30 Pa, more preferably greater than 50 Pa, such as 54 Pa, see Table 2, measured at shear rate 300 s⁻¹, measured under following conditions:

200 ml of soy milk supplemented with 2% glucose (as described in Example 2) are inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

In this embodiment, advantageously, strain DSM 25485 can be used alone and/or in combination with one or more of the LAB (i) to (x) of the present invention, described in the first aspect of the present invention. Preferably, strain DSM 25485 is used in combination with one or more of the LAB of the present invention, described in the first aspect of the present invention in a ratio of about 9:1 (LAB strain(s) of the present invention: strain DSM 25485).

In a further specific embodiment of the third aspect, the present invention provides the use of the Lactococcus lactis subsp. lactis DSM 33192, for increasing viscosity of a fermented milk product. The present inventors have surprisingly 3 found that strain DSM 33192 generated fermented milks having a shear stress greater than 40 Pa, preferably greater than 50 Pa, more preferably greater than 80 Pa, even more preferably greater than 90 Pa, such as 94 Pa, see Table 1, measured at shear rate 300 s⁻¹, measured under following conditions:

200 ml semi-fat milk (1.5% fat) is heated to 90° C. for 20 min, followed by cooling to inoculation temperature, and inoculated with 2 ml of an overnight culture of the lactic acid bacterium strain, and left at inoculation temperature until pH 4.55 followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 1.

In addition, the present inventors have surprisingly found that strain DSM 33192 generated fermented milks having a shear stress greater than 24 Pa, preferably greater than 30 Pa, more preferably greater than 40 Pa, even more preferably greater than 45 Pa, such as 47 Pa, see Table 2, measured at shear rate 300 s⁻¹, measured under following conditions:

200 ml of soy milk supplemented with 2% glucose (as described in Example 2) are inoculated with 2 ml of an overnight culture of the lactic acid bacterium strain, and left at inoculation temperature until pH 4.55, followed by storage at 4° C. until shear stress is measured, typically from 1-7 days, such as for 5 days, followed by gently stirring and measuring the shear stress at shear rate 300 s⁻¹, wherein the inoculation temperature is 30° C. The shear stress is measured using the method indicated in Example 2.

In this embodiment, advantageously, strain DSM 33192 can be used alone and/or in combination with one or more of the LAB (i) to (x) of the present invention, described in the first aspect of the present invention. Preferably, strain DSM 33192 is used in combination with one or more of the LAB of the present invention, described in the first aspect of the present invention in a ratio of about 9:1 (LAB strain(s) of the present invention: strain DSM 33192).

In a specific embodiment of the third aspect, the present invention provides the use of the Lactococcus lactis subsp. cremoris strain DSM 25485 and/or the use of Lactococcus lactis subsp. lactis strain DSM 33192, as co-acidifier or helper strains, preferably their use in combination with other texturing LAB strains, such as strains (i) to (x) as defined in the first aspect of the present invention, for increasing viscosity of a fermented milk product.

Method of Producing a Food Product and Food Product

In a fourth aspect, the present invention relates to a method of producing a food product comprising at least one stage in which at least one lactic acid bacterium strain (i) to (x) as defined in the first aspect of the present invention and/or the composition as defined in the second aspect of the present invention is used. The production of the food product is carried out by methods known to the person skilled in the art.

In another embodiment, the present invention relates to a method of producing a food product comprising at least one stage in which the lactic acid bacterium strain Lactococcus lactis subsp. cremoris DSM 25485, or a mutant or variant therefrom is used.

In another embodiment, the present invention relates to a method of producing a food product comprising at least one stage in which the lactic acid bacterium strain Lactococcus lactis subsp. lactis DSM 33192, or a mutant or variant therefrom is used.

“Fermentation” in the context of the present invention in any of its embodiments means the conversion of carbohydrates into alcohols or acids through the action of microorganisms (LAB). Fermentation processes to be used in production of food products such as dairy products are well known and the person of skill in the art will know how to select suitable process conditions, such as temperature, oxygen, amount of microorganism(s) and process time. Obviously, fermentation conditions are selected so as to support the achievement of the present invention, e.g., to obtain a food product, preferably a food product which has an improved texture as compared to a food product produced with a method which does not involve the use of at least one of the LAB as described in the first aspect of the present invention or the use of the composition as described in the second aspect of the present invention, in any of its embodiments.

In one preferred embodiment, the method of the present invention in any of its embodiments comprises fermenting a milk substrate, which can be a mammalian-based milk substrate or a plant-based milk substrate, such as soy milk, preferably supplemented with glucose, such as 0.5-5%, preferably 0.5-2%, more preferably 2%, with a composition comprising at least 1×10⁶ CFU, preferably at least 1×10⁸ CFU/ml of total LAB strains.

For instance, the method of the present invention comprises fermenting a milk substrate with a composition comprising at least 1×10⁶ CFU, preferably at least 1×10⁸ CFU/ml of strains DSM 33134 and DSM 25485.

For instance, the method of the present invention comprises fermenting a milk substrate with a composition comprising at least 1×10⁶ CFU, preferably at least 1×10⁸ CFU/ml of strains DSM 33135 and DSM 25485.

For instance, the method of the present invention comprises fermenting a milk substrate with a composition comprising at least 1×10⁶ CFU, preferably at least 1×10⁸ CFU/ml of strains DSM 33136 and DSM 25485.

For instance, the method of the present invention comprises fermenting a milk substrate with a composition comprising at least 1×10⁶ CFU, preferably at least 1×10⁸ CFU/ml of strains DSM 33138 and DSM 25485.

For instance, the method of the present invention comprises fermenting a milk substrate with a composition comprising at least 1×10⁶ CFU, preferably at least 1×10⁸ CFU/ml of strains DSM 33139 and DSM 25485.

For instance, the method of the present invention comprises fermenting a milk substrate with a composition comprising at least 1×10⁶ CFU, preferably at least 1×10⁸ CFU/ml of strain DSM 33137. For instance, the method of the present invention comprises fermenting a milk substrate with a composition comprising at least 1×10⁶ CFU, preferably at least 1×10⁸ CFU/ml of strains DSM 33137 and DSM 25485.

For instance, the method of the present invention comprises fermenting a milk substrate with a composition comprising at least 1×10⁶ CFU, preferably at least 1×10⁸ CFU/ml of strain DSM 33140. For instance, the method of the present invention comprises fermenting a milk substrate with a composition comprising at least 1×10⁶ CFU, preferably at least 1×10⁸ CFU/ml of strains DSM 33140 and DSM 25485.

For instance, the method of the present invention comprises fermenting a milk substrate with a composition comprising at least 1×10⁶ CFU, preferably at least 1×10⁸ CFU/ml of strain DSM 33142.

For instance, the method of the present invention comprises fermenting a milk substrate with a composition comprising at least 1×10⁶ CFU, preferably at least 1×10⁸ CFU/ml of strains DSM 33141 and DSM 25485.

For instance, the method of the present invention comprises fermenting a milk substrate with a composition comprising at least 1×10⁶ CFU, preferably at least 1×10⁸ CFU/ml of strain DSM 33183.

In another preferred embodiment, the method comprises fermenting a milk substrate with the composition as described in the second aspect of the present invention, in any of its embodiments.

Preferably, the food product is a dairy product and the method in any of its embodiments comprises fermenting a milk substrate (also referred to as “milk base” in the context of the present invention) with the at least one LAB strain and/or with the composition according to the invention (first and second aspects, respectively) and/or with a co-acidifier or helper strain, as defined above, preferably with strain DSM 25485 and/or with strain DSM 33192.

Preferably, the food product is a dairy product and the method in any of its embodiments comprises fermenting a plant-based milk substrate (also referred to as “plant-based milk base” in the context of the present invention), such as soy milk, preferably soy milk supplemented with sugar, with the at least one LAB strain and/or with the composition according to the invention (first and second aspects, respectively). Sugar may be such as e.g. fructose, sucrose, High Fructose Corn Syrup (HFCS), honey, glucose, invert sugar, maltose, galactose, lactose, or any combination thereof. The concentration of sugar may be between 0.5% to 5%, from 0.5 to 2%, 0.5%, 1%, 1.5%, or 2% such as e.g. 0.5-5% glucose, preferably 0.5-2% glucose, more preferably about 2% glucose.

The food product according to the present invention may advantageously further comprise a thickener and/or a stabilizer, such as pectin (e.g. HM pectin, LM pectin), gelatin, CMC, Soya Bean Fiber/Soya Bean Polymer, starch, modified starch, carrageenan, alginate, and guar gum.

In a specific embodiment the food product is a dairy product, a meat product, a vegetable product, a fruit product or a cereal product. In a preferred embodiment, the food product is a dairy product. In another preferred embodiment, the food product is a plant-based food product, such as fermented soy milk.

The term “dairy product” as used herein refers to a food product produced from milk. As described above, in the context of the present application, the term “milk” is broadly used in its common meaning to refer to liquids produced by the mammary glands of animals (e.g., cows, sheep, goats, buffaloes, camel, etc.) or by plants. In a preferred embodiment, the milk is cow's milk. In accordance with the present invention the milk may have been processed and the term “milk” includes whole milk, skim milk, fat-free milk, low fat milk, full fat milk, lactose-reduced milk, or concentrated milk. Fat-free milk is non-fat or skim milk product. Low-fat milk is typically defined as milk that contains from about 1% to about 2% fat. Full fat milk often contains 2% fat or more. The term “milk” is intended to encompass milks from different mammalians and plant sources. Mammalian sources of milk include, but are not limited to cow, sheep, goat, buffalo, camel, llama, mare and deer. Plant sources of milk include, but are not limited to, milk extracted from soy bean. In a specific embodiment, the milk is cow's milk. In another specific embodiment, the milk is a plant-based milk, preferably soy milk, which can be preferably supplemented with sugar such as e.g. fructose, sucrose, High Fructose Corn Syrup (HFCS), honey, glucose, invert sugar, maltose, galactose, lactose, or any combination thereof. The concentration of sugar may be between 0.5% to 5%, from 0.5 to 2%, 0.5%, 1%, 1.5%, or 2% such as e.g. 0.5-5% glucose, preferably 0.5-2% glucose, more preferably about 2% glucose.

Preferred dairy products according to the invention are fermented milk products and cheese. In a specific embodiment the dairy product is a mesophilic dairy product.

In a particular embodiment of the invention, the fermented milk product is selected from the group consisting of buttermilk, sour milk, cultured milk, Smetana, sour cream, thick cream, cultured cream, ymer, fermented whey, Kefir, Yakult and fresh cheese, such as Quark, tvarog, and cream cheese. In particular, the fermented milk product is selected from the group consisting of Quark, sour cream and Kefir. In a preferred embodiment of the invention, the fermented milk product contains a further food product selected from the group consisting of fruit beverage, cereal products, fermented cereal products, chemically acidified cereal products, soy milk products, fermented soy milk products and any mixture thereof. In another preferred embodiment, the fermented milk product is a plant-based fermented milk product, such as fermented soy milk (e.g. plantgurt, from “Alpro”).

The fermented milk product typically contains protein in a level of between 1.0% by weight to 12.0% by weight, preferably between 2.0% by weight to 10.0% by weight. In a particular embodiment, sour cream contains protein in a level of between 1.0% by weight to 5.0% by weight, preferably between 2.0% by weight to 4.0% by weight. In a particular embodiment, Quark contains protein in a level of between 4.0% by weight to 12.0% by weight, preferably between 5.0% by weight to 10.0% by weight.

Preferably, the food product has an improved texture (improved viscosity, measured as shear stress at 300 s⁻¹, as described in the present invention and, e.g., in Example 1) as compared to a food product produced with a comparable method which does not involve the use of at least one of the LAB as described in the first aspect of the present invention and/or the use of the composition as described in the second aspect of the present invention, in any of its embodiments and/or the use of strain DSM 25485 and/or DSM 33192.

The invention also relates to a food product, preferably a dairy product, comprising at least one LAB strain as described in the first aspect of the present invention.

Any combination of the above-described elements, aspects and embodiments in all possible variations thereof is encompassed by the invention unless otherwise indicated herein or otherwise clearly contradicted by context. Embodiments of the present invention are described below, by way of examples only.

### EXAMPLES

### Example 1. High-Throughput Screening for Texturing Strains and Measurement of Milk Gel Texture

L. lactis is used to produce numerous fermented dairy products including cheese and mesophilic fermented milk, such as buttermilk and sour cream. Polysaccharide-producing strains are of great interest for these applications, as polysaccharides released into the medium can result in improved texturing properties of buttermilk and sour cream, while capsular polysaccharides can result in improved water-holding capacity and thus improved yields of, e.g., cheese.

Milk (liquid) is typically converted into milk gel (soft solid) when fermented with lactic acid bacteria typically belonging to, e.g., Streptococcus thermophilus, Lactobacillus spp. and Lactococcus lactis spp. Rheometer or texture analyzer are typically used to assess rheological properties of fermented milk gels, such as shear stress. Shear stress measurements are related to perceived mouth thickness, when the texture of milk gels is assessed by a sensory panel. High mouth thickness is considered an important quality factor of fermented milk gels such as yoghurt, and consumer acceptance is often very closely linked to the texture properties such as mouth thickness, which is a function of shear stress.

A liquid handling unit, Hamilton Robotics MicroLab Star, equipped with pressure sensor inside the air displacement barrel of the individual pipettes was used to screen for texturing strains as described in Poulsen et al., 2019. The liquid handler has a pressure sensor located in the headspace of each pipetting channel. Pressure data from each sensor was collected by TADM (Total Aspiration Dispense Monitoring) software of the Hamilton Robotics MicroLab Star liquid handler (Hamilton Robotics) and used to assess the relative shear stress of milk gel samples.

L. lactic from high-throughput screening strain library were screened for texturing properties using the TADM tool of the Hamilton liquid handling robot, as stated above, in 2-ml scale. Pressure versus time data (TADM) were obtained from 2 ml samples made in a 96-well micro-titer plate, where B-milk was inoculated for 20 h at 30° C. in the presence of different strains (1% of inoculum) unless otherwise stated, and then stored at 4 QC for 1 day. Hamilton liquid handling unit was used to measure pressure during aspiration, and the area above the pressure curves obtained during aspiration was used to compare the texturing abilities of the strains.

Shear stress data were obtained by inoculating the same microbial cultures in semi-fat milk (1.5% fat); milk was heated at 90° C. for 20 min and cooled down to the inoculation temperature, prior to inoculation with 1% volume overnight microbial culture. The inoculation took place for 8-22 h at 30° C. in 200-ml scale until pH^{˜}4.55, followed by cooling to 4° C. and storage until shear stress is measured, typically from 1-7 days, such as for 5 days at 4° C. After the storage, the fermented milk was stirred gently by means of a stick fitted with a bored disc until homogeneity of the sample. Shear stress of the samples was assessed on a rheometer (Anton Paar Physica Rheometer with ASC, Automatic Sample Changer, Anton Paar® GmbH, Austria) using the following settings:
Wait time (to rebuild to somewhat original structure)
5 minutes without oscillation or rotation
Rotation (to measure shear stress at 300 s⁻¹ etc.)
Y′=[0.2707-300] s⁻¹ and y′=[275-0.2707] s⁻¹

21 measuring points over 210 s (on every 10 s) going up to 300 s⁻¹ and 21 measuring points over 210 s (one every 10 s) going down to 0.2707 s⁻¹. For the data analysis, the shear stress at shear rate 300 s⁻¹ was chosen.

The good texturing ability of 11 strains DSM 33134, 33135, DSM 33136, 33137, 33138, 33139, 33140, 33141, 33142, 33183 and 33192 was confirmed using the rheometer as described above. Further, the good texturing ability of strain DSM 25485 was also confirmed using the rheometer as described above. The results are shown in Table 1.

TABLE 1
Shear stress and time-to-pH 4.55 for selected texturing strains (1% of inoculum) ±
yeast extract (0.2%, see below for the details) ± potential co-acidifier
(DSM 24649 or DSM 25485) incubated in heat-treated semi-fat milk at 30° C.,
as described above. Some of the strains under investigation are slow
acidifiers and require either presence of yeast
extract or a co-acidifier strain to acidify milk in an acceptable time, e.g.,
15 h or less to reach a
pH of 4.55. Strains DSM 29291, DSM 24649 and DSM 25485 were used alone or
in combination with strains under investigation and/or as potential
co-acidifiers (10 % inoculum—which corresponds to a ratio 9:1 (selected texturing
strain: co-acidifier strain). Generally, 1% inoculum of the strains
in milk (i.e. 2 ml overnight inoculum in
M17 medium added to 200 ml milk) was used. When a single strain is used as
inoculum, 2 ml (100 %) of this strain are added to 200 ml milk (1 % inoculum
in milk). When a strain is used together with a co-acidifier, 90% of
the strain under investigation (1.8 ml) and 10% (0.2 ml) of a co-acidifier are used
(10% of inoculum for the co-acidifier, ratio 9:1 (selected
texturing strain : co-acidifier strain).

| DSMZ | | | | | | | | | | |
|---|---|---|---|---|---|---|---|---|---|---|
| depos. | Shear stress, Pa | | | | | Time-to-pH 4.55, h | | | | |
| nr. | alone | +24649 | +25485 | +33192 | +YE | alone | +24649 | +25485 | +33192 | +YE |
| 24649 | 20 | | | | | 8 | | | | |
| 25485 | 56 | | | | | 12 | | | | |
| 33192 | 94 | | | | 88 | 14 | | | | 12 |
| 29291 | 39 | | 40 | 52 | 45 | 11 | | 12 | 12 | 8 |
| 33134 | 46 | 45 | 63 | | 53 | 18 | 13 | 13 | | 7 |
| 33135 | 43 | 28 | 65 | | 36 | 18 | 10 | 11 | | 8 |
| 33136 | 41 | 24 | 60 | | 37 | 18 | 11 | 12 | | 8 |
| 33137 | 48 | 39 | 48 | | 56 | 10 | 10 | 11 | | 8 |
| 33138 | 55 | 26 | 67 | | 36 | 18 | 10 | 11 | | 8 |
| 33139 | 2 | 42 | 52 | | 53 | 18 | 10 | 10 | | 7 |
| 33140 | 46 | 25 | 64 | | 44 | 15 | 10 | 11 | | 10 |
| 33142 | 56 | | | | | 13 | | | | |
| 33141 | | | 41 | | 38 | | | 15 | | 8 |
| 33183 | 72 | | | | | 18 | | | | |

The following texturing strains were classified as “slow acidifiers”, as they were able to reach pH 4.55 in more than 15 h, measured as described above, when grown alone (Table 1): DSM 33134, 33135, DSM 33136, 33138, 33139 and 33141. Yeast extract (YE, 0.2%) or a co-acidifier strain (DSM 24649 or DSM 25485) were added to the milk base to help the slow-acidifying strains to grow in milk. Yeast extract was obtained from Procelys (Yeast Extract NuCel® 545 MG, batch number 0005115910, batch AD 18 A05030).

Yeast extract can enhance the acidification speed, but it had a negative effect on the texture development when co-incubated with some of the strains under investigation, e.g., DSM 33135, DSM 33136, 33138. DSM 33138 lost ⅓ of its texture (expressed as shear stress) when yeast extract was present.

The aim with adding co-acidifier strains was to help the texturing strains to acidify the milk quicker, namely to reach a pH of about 4.55 in 15 h or less, and to not influence their texture negatively, as we often see in the presence of yeast extract. Also, different polysaccharides produced by two or more different strains grown together might have a synergistic effect on texture. For instance, DSM 33135 grown in the presence of 10% DSM 25485, as described above, did both result in an enhanced acidification speed but also in an enhanced texture: shear stress of a combination of DSM 33135 and DSM 25485 was significantly higher than that of DSM 25485 or DSM 33135 alone. Similarly shear stress of a combination of DSM 33140 and DSM 25485 was significantly higher than that of DSM 25485 or DSM 33140 alone. DSM 33137 had similar behavior in the presence of DSM 25485 (Table 1). As DSM 33134, 33135, DSM 33136, 33138, 33139 and 33141 are slow acidifiers when fermenting milk on their own (it took more than 15 h to reach a pH of 4.55, as described above), it was advantageous to test them in the presence of a co-acidifier strain, which would help to reduce the acidification time and, at the same time, would not negatively influence the generated viscosity, measured as shear stress, as in Table 1.

### Example 2. Rheology Measurement of Texture in Soy Milk

Rheology measurements of 14 L. lactic strains in soy milk supplemented with 2% glucose were performed.

The strains tested in this example were the following: DSM 24649 (non-texturing); DSM 33134; DSM 33135; DSM 33136; DSM 33137; DSM 33138; DSM 33139; DSM 33140; DSM 33141; DSM 33142; DSM 33183; DSM 33192; and DSM 25485.

The milk base used in was soy milk supplemented with 2% glucose: The soy milk was organic and unsweetened, obtained from Naturli′ Foods, with the following composition per 100 ml:

Fat: 2.1 g
Thereof saturated fat: 0.4 g

Carbohydrates: 0.1 g
Thereof sugars: 0.1 g

Fibers: 0.6 g

Protein: 3.7 g

Salt: 0.04 g.

The milk was already sterile, and no pre-treatment was performed to it before its use. It was supplemented with 2% glucose.

1% volume overnight microbial culture (obtained by inoculating the microbial cultures in M17 broth supplemented with 2% glucose at 30° C.) was inoculated in soy milk with 2% glucose. The inoculation took place at 30° C. in 200-ml scale until pH^{˜}4.55 (see Table 3 for the specific pH reached by each culture), followed by cooling to 4° C. and storage until shear stress is measured, typically from 1-7 days, such as for 5 days. After the storage, the fermented milk was stirred gently by means of a stick fitted with a bored disc until homogeneity of the sample. Shear stress of the samples was assessed on a rheometer (Anton Paar Physica Rheometer with ASC, Automatic Sample Changer, Anton Paar® GmbH, Austria) using the following settings:
Wait time (to rebuild to somewhat original structure)
5 minutes without oscillation or rotation
Rotation (to measure shear stress at 300 s⁻¹ etc.)
Y′=[0.2707−300] s⁻¹ and y′=[275−0.2707] s⁻¹

21 measuring points over 210 s (on every 10 s) going up to 300 s′ and 21 measuring points over 210 s (one every 10 s) going down to 0.2707 s⁻¹. For the data analysis, the shear stress at shear rate 300 s⁻¹ was chosen.

The results of the shear stress (Pa) are shown in Table 2 below. “Alpro” refers to “Alpro naturell mild & creamy plantgurt”, a commercially-available fermented soy milk from “Al pro” (www.alpro.conn/se/produkter/vaxtbaserad-yoghurt-variant/nnild-creanny/nnild-creamy-naturell/), with the following composition per 100 ml:

| Energy | 210 kJ/50 kcal | |
|---|---|---|
| Fatt | 2.3 | g |
| Saturated fatty acids | 0.4 | g |
| Monounsaturated | 0.5 | g |
| Polyunsaturated | 1.4 | g |
| Carbohydrates | 2.1 | g |
| Sugar | 2.1 | g |
| Fibers | 1 | g |
| Protein | 4 | g |
| Salt | 0.3 | g |
| Vitamins | | |
| Vitamin D | 0.75 | μg |
| Vitamin B12 | 0.38 | μg |
| Minerals | | |
| Calcium | 120 | mg |

And with the following ingredients: Water, peeled SOYBEANS (7.9%), sugar, tricalcium citrate, stabilizer (pectin), acidity regulators (sodium citrate, citric acid), sea salt, antioxidants (tocopherol-rich extract, ascorbic acid esters of edible fatty acids), vitamins (B12, D2), yogurt culture (S. thermophilus, L. bulgaricus). Of note, Alpro comprises pectin, which increases the texture of the fermented milk. However, the base milk used in this example (soy milk supplemented with 2% glucose) does not include pectin.

TABLE 2
Shear stress (Pa) at several shear rates (s⁻¹) for selected texturing strains (1% of inoculum) incubated
in soy milk with 2% glucose, as described above.

| | Shear stress (Pa) at shear rate (s⁻¹) | | | | | | | | | | | | | | | | | | | | |
|---|---|---|---|---|---|---|---|---|---|---|---|---|---|---|---|---|---|---|---|---|---|
| Sample | 0,27 | 15 | 30 | 45 | 60 | 75 | 90 | 105 | 120 | 135 | 150 | 165 | 180 | 195 | 210 | 225 | 240 | 255 | 270 | 285 | 300 |
| Alpro | 17 | 36 | 40 | 42 | 44 | 46 | 47 | 48 | 49 | 50 | 51 | 52 | 53 | 54 | 55 | 56 | 56 | 57 | 58 | 60 | 60 |
| DSM 24649 | 7 | 16 | 18 | 19 | 20 | 20 | 21 | 21 | 21 | 21 | 22 | 22 | 22 | 23 | 23 | 23 | 23 | 24 | 24 | 24 | 24 |
| DSM 33134 | 6 | 18 | 21 | 24 | 27 | 28 | 30 | 31 | 32 | 32 | 33 | 33 | 34 | 34 | 35 | 35 | 35 | 36“ | 36 | 37 | 37 |
| DSM 33135 | 6 | 18 | 20 | 22 | 24 | 25 | 25 | 26 | 26 | 27 | 27 | 27 | 28 | 28 | 28 | 28 | 28 | 29 | 29 | 29 | 30 |
| DSM 33136 | 6 | 16 | 19 | 20 | 22 | 23 | 24 | 24 | 25 | 25 | 26 | 26 | 27 | 27 | 27 | 28 | 28 | 29 | 29 | 29 | 29 |
| DSM 33137 | 5 | 16 | 18 | 20 | 22 | 24 | 26 | 27 | 28 | 29 | 30 | 30 | 31 | 31 | 32 | 32 | 33 | 34 | 34 | 34 | 35 |
| DSM 33138 | 6 | 15 | 17 | 19 | 20 | 21 | 22 | 23 | 23 | 24 | 24 | 24 | 25 | 25 | 25 | 26 | 26 | 26 | 27 | 27 | 27 |
| DSM 33139 | 5 | 17 | 20 | 23 | 26 | 29 | 31 | 32 | 34 | 35 | 36 | 37 | 37 | 38 | 38 | 39 | 40 | 40 | 41 | 41 | 42 |
| DSM 33140 | 5 | 16 | 18 | 19 | 20 | 21 | 22 | 23 | 24 | 24 | 24 | 25 | 25 | 26 | 26 | 26 | 27 | 27 | 28 | 28 | 28 |
| DSM 33141 | 5 | 15 | 18 | 20 | 22 | 23 | 25 | 26 | 26 | 27 | 27 | 28 | 28 | 29 | 29 | 30 | 30 | 31 | 31 | 31 | 32 |
| DSM 33142 | 5 | 15 | 19 | 22 | 24 | 27 | 29 | 31 | 32 | 34 | 35 | 36 | 37 | 37 | 38 | 39 | 39 | 40 | 41 | 41 | 42 |
| DSM 33183 | 5 | 18 | 25 | 31 | 36 | 40 | 43 | 46 | 48 | 50 | 52 | 53 | 55 | 56 | 56 | 57 | 57 | 58 | 58 | 59 | 59 |
| DSM 25485 | 6 | 17 | 25 | 30 | 34 | 38 | 41 | 43 | 45 | 47 | 48 | 49 | 50 | 51 | 52 | 52 | 53 | 53 | 54 | 54 | 54 |
| DSM 33192 | 5 | 16 | 21 | 25 | 29 | 32 | 35 | 37 | 39 | 40 | 41 | 42 | 43 | 43 | 44 | 45 | 46 | 46 | 46 | 47 | 47 |

As it can be seen from Tables 2, all selected texturing strains showed higher shear stress when fermenting soy milk supplemented with glucose 2% than the negative control (DSM24649).

Finally, the pH reached by each of the strains, and the time to this pH (in h) is shown in Table 3.

TABLE 3
Time-to-pH for selected texturing strains (1% of inoculum) incubated
in soy milk supplemented with 2% glucose, as described above.

| Strain (DSM) | pH reached | Time to pH, h |
|---|---|---|
| Alpro | | |
| DSM 24649 + 2% Glc | 4.55 | 8 |
| DSM 33134 + 2% Glc | 4.56 | 20.5 |
| DSM 33135 + 2% Glc | 4.68 | 21.5 |
| DSM 33136 + 2% Glc | 4.64 | 20.5 |
| DSM 33137 + 2% Glc | 4.40 | 8 |
| DSM 33138 + 2% Glc | 4.71 | 21.5 |
| DSM 33139 + 2% Glc | 4.58 | 15.5 |
| DSM 33140 + 2% Glc | 4.55 | 11 |
| DSM 33141 + 2% Glc | 4.58 | 16 |
| DSM 33142 + 2% Glc | 4.55 | 12 |
| DSM 33183 + 2% Glc | 4.86 | 20.5 |
| DSM 25485 + 2% Glc | 4.55 | 16 |
| DSM 33192 + 2% Glc | 4.55 | 14 |

### Example 3. Sequencing Genomes of Lactococcal Strains

The genome of the strains was sequenced in-house at Chr. Hansen as described by Agersø et al. (Agersoe et al., 2018). In brief, total DNA was purified and used to prepare a 250-bp paired-end library for genome sequencing using Illumina MiSeq system. The sequence reads were subjected to quality trimming (Phred score<25) and assembled into contigs using the de novo assembly algorithm in CLC Genomics Workbench, version 10.1.1 (CLC bio, Qiagen Bioinformatics). The resulting genome assembly was filtered by removing contigs with coverage of <15X and/or <20% of the median coverage of the assembly. The consensus sequence of the remaining contigs was exported in FASTA format, which is referred to as the draft genome sequence, and used in the subsequent sequence analysis.

### Example 4. Characterization of the Eps Gene Clusters of the Lactococcal Strains

Since an enhanced texture is associated with the production of polysaccharides, mining for eps gene clusters was performed. Mobile genetic elements (IS elements also called transposases) flanking or within the operon are consistently present in the architecture of eps gene clusters, but they are not involved in the polysaccharide biosynthesis. The biosynthesis of exocellular polysaccharides in L. lactic occurs via the Wzy-dependent pathway. Here, we used the nomenclature suggested by Zeidan et al. (2017). The conserved genes in the beginning of the eps gene cluster were denominated epsRXCDB, and those at the end, epsL and lytR, while the polymerase was named wzy, and the flippase, wzx.

Genes located at the 5′ end of the eps gene cluster epsRXCDB, which are involved in the modulation and assembly machinery of polysaccharide biosynthesis, as well as epsL and lytR at the 3′ end, displayed the highest level of conservation. The genes of the variable part including polymerase wzy, flippase wzx, and glucosyltransferases (GT) or other polymer-modifying enzymes, were rarely similar between the strains. The common denominator for the texturing strains is that they all contain the genes required for the polysaccharide production, e.g. epsCDBE-wzy-wzx and GT (Zeidan et al. 2017).

The eps gene cluster of DSM 33134 is 18254 bp long and contains 20 open reading frames (ORF) corresponding to eight conserved genes (epsRXCDBEL, lytR) and 12 genes of the variable part. Genes of the variable part likely involved in polysaccharide biosynthesis include three glycosyltransferases (GT1, GT2, GT3), nucleotide sugar dehydrogenase, polymerase wzy, and flippase (polysaccharide transporter) wzx. The three GT together with a putative nucleotide sugar dehydrogenase are potentially involved in sequential building of the repeating unit, although their specific functions and therefore order of action have not been demonstrated. Five IS elements are also a part of the eps gene cluster of DSM 33134.

The eps gene cluster of DSM 33135 is 27444 bp long and contains 31 ORF corresponding to eight conserved genes (epsRXCDBEL, lytR) and 19 genes of the variable part. Genes of the variable part likely involved in polysaccharide biosynthesis include five glycosyltransferases (GT1, GT2, GT3, GT4, GT5), dTDP-glucose 4,6-dehydratase, dTDP-4-dehydrorhamnose reductase, dTDP-4-dehydrorhamnose 3,5-epimerase, DUF1919, DUF4422, UDP-galactopyranose mutase, polymerase wzy, and flippase (polysaccharide transporter) wzx. Nine IS elements are also a part of the eps gene cluster of DSM 33135. VanZ family protein is usually found in the eps gene clusters of S. thermophilus, but not in L. lactis.

The eps gene cluster of DSM 33136 is 18365 bp long and contains 18 ORF corresponding to eight conserved genes (epsRXCDBEL, lytR) and 10 genes of the variable part. Genes of the variable part likely involved in polysaccharide biosynthesis include three glycosyltransferases (GT1, GT2, GT3), polysaccharide pyruvyl transferase, polymerase wzy, and flippase (polysaccharide transporter) wzx. The three GT together with a putative polysaccharide pyruvyl transferase are potentially involved in sequential building of the repeating unit, although their specific functions and therefore order of action have not been demonstrated. Two IS elements are also a part of the eps gene cluster of DSM 33136. VanZ family protein is usually found in the eps gene clusters of S. thermophilus, but not in L. lactis.

The eps gene cluster of DSM 33137 is 20584 bp long and contains 21 ORF corresponding to eight conserved genes (epsRXCDBEL, lytR) and 13 genes of the variable part. Genes of the variable part likely involved in polysaccharide biosynthesis include four glycosyltransferases (GT1, GT2, GT3, GT4), Cre-2/I-branching protein, polymerase wzy, and flippase (polysaccharide transporter) wzx. Five IS elements are also a part of the eps gene cluster of DSM 33137. VanZ family protein is usually found in the eps gene clusters of S. thermophilus, but not in L. lactis.

The eps gene cluster of DSM 33138 is 21315 bp long and contains 23 ORF corresponding to seven conserved genes (epsRXCDBE, lytR) and 16 genes of the variable part. Genes of the variable part likely involved in polysaccharide biosynthesis include four glycosyltransferases (GT1, GT2, GT3, GT4), DUF1972, DUF4422, DUF1919, UDP-galactopyranose mutase, dTDP-4-dehydrorhamnose 3,5-epimerase, dTDP-glucose 4,6-dehydratase, dTDP-4-dehydrorhamnose reductase, polymerase wzy, and flippase (polysaccharide transporter) wzx. Three IS elements are also a part of the eps gene cluster of DSM 33138. VanZ family protein is usually found in the eps gene clusters of S. thermophilus, but not in L. lactis.

The eps gene cluster of DSM 33139 is 27175 bp long and contains 29 ORF corresponding to eight conserved genes (epsRXCDBEL, lytR) and 21 genes of the variable part. Genes of the variable part likely involved in polysaccharide biosynthesis include six glycosyltransferases (GT1, GT2, GT3, GT4, GT5, GT6), two NAD-dependent epimerases, nucleotide sugar dehydrogenase, RfbA, dTDP-glucose 4,6-dehydratase, dTDP-4-dehydrorhamnose 3,5-epimerase, two acyltransferases, dTDP-4-dehydrorhamnose reductase, nucleotidyltransferase, polymerase wzy, and flippase (polysaccharide transporter) wzx. VanZ family protein is also a part of the eps gene cluster of DSM 33139; it is usually found in the eps gene clusters of S. thermophilus, but not in L. lactis.

The eps gene cluster of DSM 33140 is 18226 bp long and contains 19 ORF corresponding to eight conserved genes (epsRXCDBEL, lytR) and 11 genes of the variable part. Genes of the variable part likely involved in polysaccharide biosynthesis include three glycosyltransferases (GT1, GT2, GT3), UDP-N-acetylglucosamine-LPS N-acetylglucosamine transferase, Capsule biosynthesis protein CapC, polymerase wzy and a second polymerase-like sequence wzy1, which is likely to short to be functional, and flippase (polysaccharide transporter) wzx. Three IS elements are also a part of the eps gene cluster of DSM 33140.

The eps gene cluster of DSM 33141 is 24364 bp long and contains 25 ORF corresponding to nine conserved genes (epsRXCDBE1E2L, lytR) and 16 genes of the variable part. Genes of the variable part likely involved in polysaccharide biosynthesis include six glycosyltransferases (GT1, GT2, GT3, GT4, GT5, GT6), acetyltransferase, nucleotide sugar dehydrogenase, acyltransferase, polymerase wzy, and flippase (polysaccharide transporter) wzx. Two IS elements are also a part of the eps gene cluster of DSM 33141.

The eps gene cluster of DSM 33142 is 16953 bp long and contains 19 ORF corresponding to nine conserved genes (epsRXCDBE1E2L, lytR) and 10 genes of the variable part. Genes of the variable part likely involved in polysaccharide biosynthesis include three glycosyltransferases (GT1, GT2, GT3), nucleotide sugar dehydrogenase, acetyltransferase, polymerase wzy, and flippase (polysaccharide transporter) wzx. Three IS elements are also a part of the eps gene cluster of DSM 33142.

The eps gene cluster of DSM 33183 is 16476 bp long and contains 19 ORF corresponding to eight conserved genes (epsRCDBE1E2L, lytR) and 10 genes of the variable part. Genes of the variable part likely involved in polysaccharide biosynthesis include three glycosyltransferases (GT1, GT2, GT3), nucleotide sugar dehydrogenase, acetyltransferase, polymerase wzy, and flippase (polysaccharide transporter) wzx. Three IS elements are also a part of the eps gene cluster of DSM 33183.

BLAST analysis was used to make annotations of open reading frames (ORF) of the strains from the CHCC culture collection. Comparative analysis of the eps gene clusters was performed using cd-hit tool (weizhongli-lab.org/cd-hit/). The % identity of sequences was calculated using percent identity matrix by Clustal Omega (www.ebi.ac.uk/Tools/msa/clustalo/).

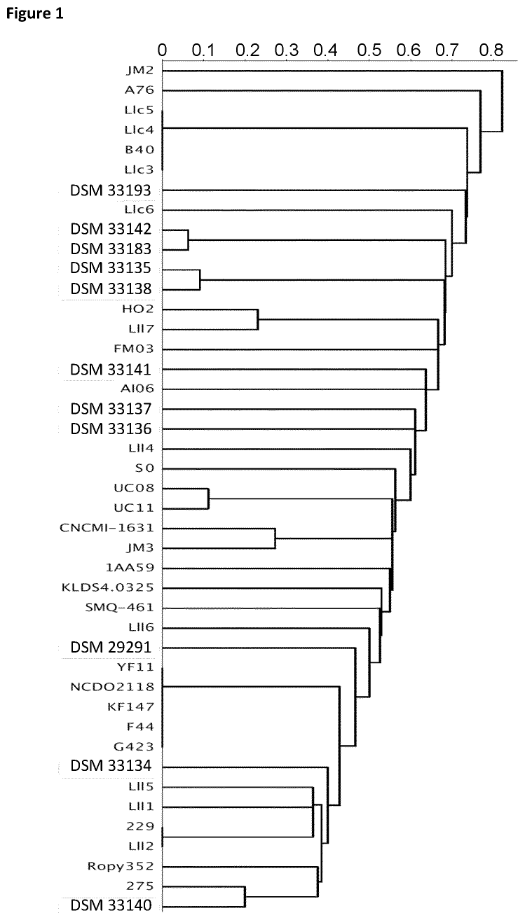

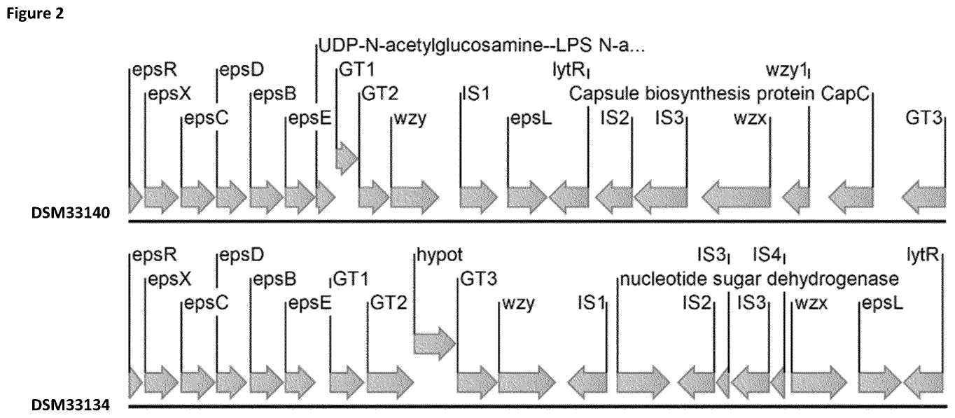

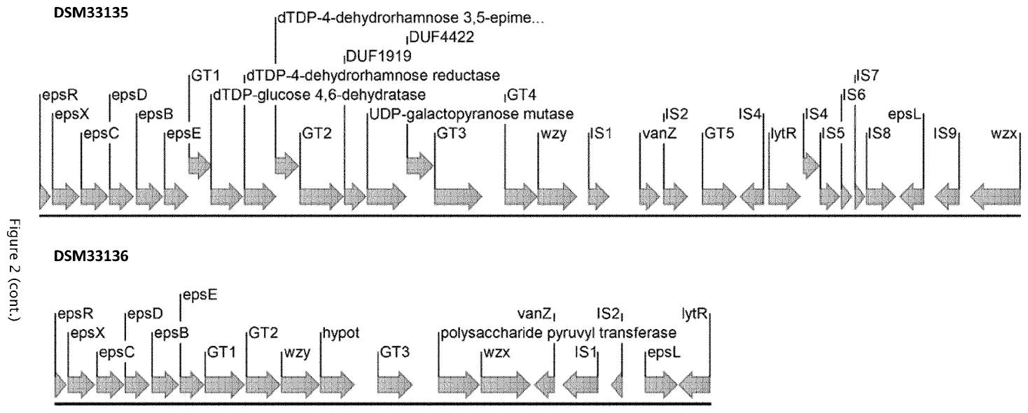

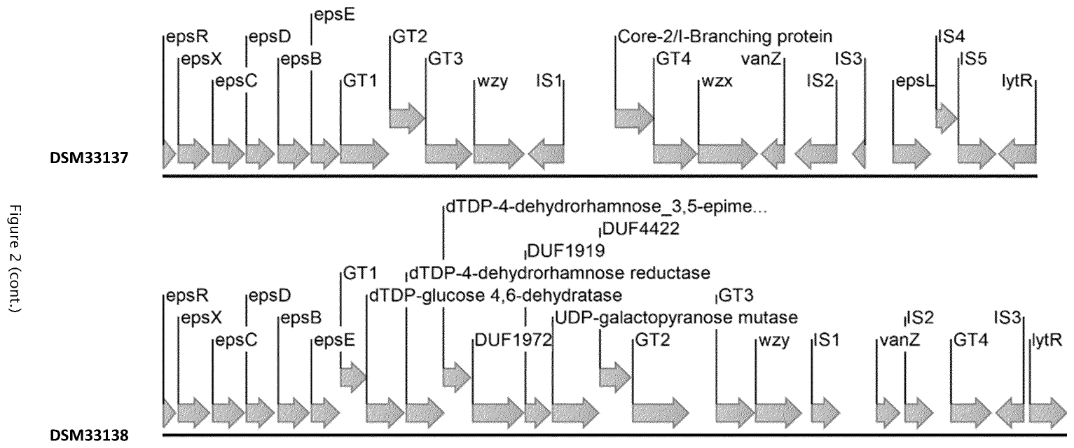

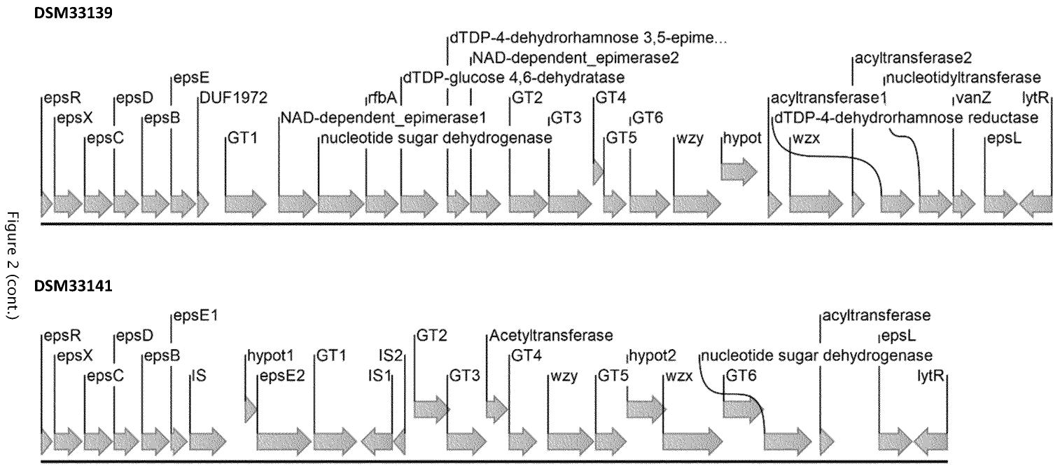

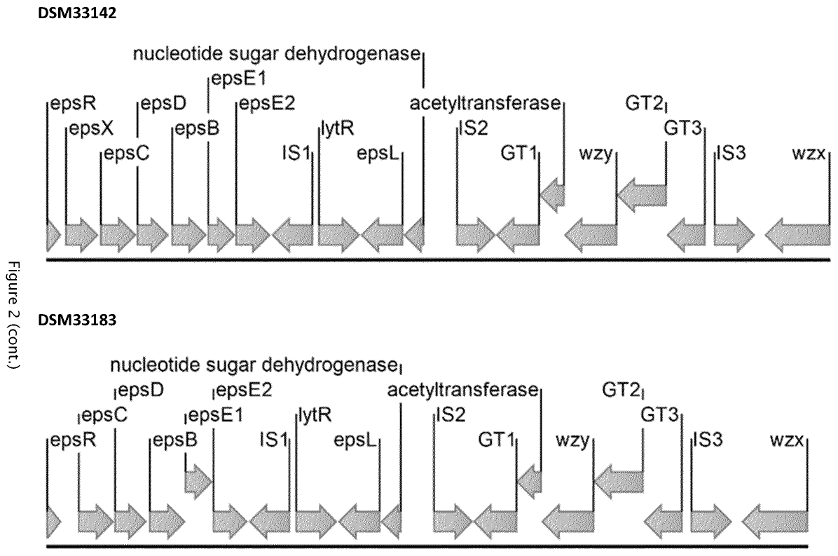

The novel texturing strains have eps gene clusters which are not similar to those in the literature or on the NCBI web-site. Also, the eps gene clusters are unique compared to the known texturing strains from the Chr. Hansen culture collection (FIG. 1).

Deposit and Expert Solution

The Applicant requests that a sample of the deposited microorganisms stated below may only be made available to an expert, until the date on which the patent is granted. In particular, the Applicant requests that the availability of the deposited microorganism referred to in Rule 33 EPC shall be effected only by the issue of a sample to an independent expert nominated by the requester (Rule 32(1) EPC).

TABLE 4
Deposits made by the Applicant, CHR. HANSEN A/S, at a
Depositary Institution having acquired the status of
International Depositary Authority under the Budapest
Treaty on the International Recognition of the Deposit
of Microorganisms for the Purposes of Patent Procedure:
Leibniz Institute DSMZ-German Collection of Microorganisms
and Cell Cultures Inhoffenstr. 7B, 38124 Braunschweig, Germany.

| Strain | Accession No. | Deposit date |
|---|---|---|
| Lactococcus lactis subsp. cremoris | DSM 25485 | Dec. 14, 2011 |
| Lactococcus lactis subsp. cremoris | DSM 24649 | Mar. 15, 2011 |
| Lactococcus lactis subsp. lactis | DSM 29291 | Aug. 21, 2014 |
| Lactococcus lactis | DSM 33134 | May 28, 2019 |
| Lactococcus lactis | DSM 33135 | May 28, 2019 |
| Lactococcus lactis | DSM 33136 | May 28, 2019 |
| Lactococcus lactis | DSM 33137 | May 28, 2019 |
| Lactococcus lactis | DSM 33138 | May 28, 2019 |
| Lactococcus lactis | DSM 33139 | May 28, 2019 |
| Lactococcus lactis | DSM 33140 | May 28, 2019 |
| Lactococcus lactis | DSM 33141 | May 28, 2019 |
| Lactococcus lactis | DSM 33192 | Jul. 3, 2019 |
| Lactococcus lactis | DSM 33142 | May 28, 2019 |
| Lactococcus lactis | DSM 33183 | Jun. 19, 2019 |
| Lactococcus lactis | DSM 33201 | Jul. 3, 2019 |
| Lactococcus lactis | DSM 33133 | May 28, 2019 |
| Lactococcus lactis | DSM 33225 | Jul. 3, 2019 |
| Lactococcus lactis | DSM 33222 | Jul. 3, 2019 |
| Lactococcus lactis | DSM 33203 | Jul. 3, 2019 |
| Lactococcus lactis | DSM 33200 | Jul. 3, 2019 |
| Lactococcus lactis | DSM 33193 | Jul. 3, 2019 |
| Lactococcus lactis | DSM 33196 | Jul. 3, 2019 |
| Lactococcus lactis | DSM 33197 | Jul. 3, 2019 |
| Lactococcus lactis | DSM 33204 | Jul. 3, 2019 |
| Lactococcus lactis | DSM 33205 | Jul. 3, 2019 |
| Lactococcus lactis | DSM 33220 | Jul. 3, 2019 |
| Lactococcus lactis | DSM 33221 | Jul. 3, 2019 |
| Lactococcus lactis | DSM 33218 | Jul. 3, 2019 |
| Lactococcus lactis | DSM 33219 | Jul. 3, 2019 |
| Lactococcus lactis | DSM 33224 | Jul. 3, 2019 |
| Lactococcus lactis | DSM 33202 | Jul. 3, 2019 |
| Lactococcus lactis | DSM 33195 | Jul. 3, 2019 |
| Lactococcus lactis | DSM 33194 | Jul. 3, 2019 |
| Lactococcus lactis | DSM 33226 | Jul. 3, 2019 |
| Lactococcus lactis | DSM 33223 | Jul. 3, 2019 |

### REFERENCES

1. Zeidan et al., 2017, Polysaccharide production by lactic acid bacteria: from genes to industrial applications. FEMS Microbiol Rev 41: 168-200.
2. Bentley et al., 2006, Genetic analysis of the capsular biosynthetic locus from all 90 pneumococcal serotypes, PLoS Genet. 2:e31.
3. Dabour and LaPointe, 2005, Identification and Molecular Characterization of the Chromosomal Exopolysaccharide Biosynthesis Gene Cluster from Lactococcus lactis subsp. cremoris SMQ-461, Appl Environ Microbiol., 71: 7414-7425.
4. Kleerebezem M., et al., 2002, Metabolic engineering of Lactococcus lactis: the impact of genomics and metabolic modelling, Journal of Biotechnology 98 (2002) 199-213.
5. Nierop Groot M. N., Kleerebezem M., 2007, Mutational analysis of the Lactococcus lactis NIZO B40 exopolysaccharide (EPS) gene cluster: EPS biosynthesis correlates with unphosphorylated EpsB, J Appl Microbiol., 103: 2645-2656.
6. Pan D., Mei X., 2010, Antioxidant activity of an exopolysaccharide purified from Lactococcus lactis subsp. lactis 12, Carbohydrate Polymers, 80: 908-914.
7. Poulsen et al., 2019, High-throughput screening for texturing Lactococcus strains, FEMS Microbiol Lett., 366(2).
8. Suzuki C. et al., 2013, Novel exopolysaccharides produced by Lactococcus lactis subsp. lactis, and the diversity of epsE genes in the exopolysaccharide biosynthesis gene clusters, Biosci Biotechnol Biochem., 77: 2013-2018.
9. Tangyu et al., 2019, Fermentation of plant-based milk alternatives for improved flavour and nutritional value, Applied Microbiology and Biotechnology, 103:9263-9275.
10. van Kranenburg R. et al., 1999, Functional analysis of glycosyltransferase genes from Lactococcus lactis and other gram-positive cocci: complementation, expression, and diversity, J Bacteriol., 181: 6347-6353.
11. Whittall J. J. et al., 2015, Topology of Streptococcus pneumoniae CpsC, a Polysaccharide co-polymerase and BY-kinase adaptor protein, J Bacteriol 197: 120-127.
12. Agersoe Y et al. (2018) Antimicrobial susceptibility testing and tentative epidemiological cutoff values for five bacillus species relevant for use as animal feed additives or for plant protection, Appl Environ Microbiol, 84 (19).

1. A Lactococcus lactis lactic acid bacterium strain comprising an active eps gene cluster capable of producing exopolysaccharide (EPS), wherein the eps gene cluster comprises nucleotide sequences selected from (i) to (x):
(i) (a): a nucleotide sequence encoding a polypeptide having polymerase activity and having at least 95% identity with the amino acid sequence encoded by the nucleotide sequence of SEQ ID NO: 11 (herein termed wzy);
(b): a nucleotide sequence encoding a polypeptide having polysaccharide transporter activity and having at least 95% identity with the amino acid sequence encoded by the nucleotide sequence of SEQ ID NO: 17 (herein termed wzx); and
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 9 (herein termed GT1);
(c2): a nucleotide sequence having at least 95% identity with the amino acid sequence encoded by the nucleotide sequence of SEQ ID NO: 13 (herein termed GT2); and
(c3): a nucleotide sequence having at least 95% identity with the amino acid sequence encoded by the nucleotide sequence of SEQ ID NO: 15 (herein termed GT3);
(ii) (a): a nucleotide sequence encoding a polypeptide having polymerase activity and having at least 95% identity with the amino acid sequence encoded by nucleotides 13939-15042 of SEQ ID NO:199 (herein termed wzy);
(b): a nucleotide sequence encoding a polypeptide having polysaccharide transporter activity and having at least 95% identity with the amino acid sequence encoded by nucleotides 26029-27444 of the complementary strand of SEQ ID NO:199 (herein termed wzx); and
(c): a nucleotide sequence encoding a polypeptide having glycosyltransferase (GT) activity comprising:
(c1): a nucleotide sequence having at least 95% identity with the amino acid sequence encoded by nucleotides 4174-4824 of SEQ ID NO:199 (herein termed GT1);
(c2): a nucleotide sequence having at least 95% identity with the amino acid sequence encoded by nucleotides 7276-8508 of SEQ ID NO:199(herein termed GT2);
(c3): a nucleotide sequence having at least 95% identity with the amino acid sequence encoded by nucleotides 11042-12391 of SEQ ID NO:199 (herein termed GT3);
(c4): a nucleotide sequence having at least 95% identity with the amino acid sequence encoded by nucleotides 13008-13934 of SEQ ID NO:199 (herein termed GT4); and
(c5): a nucleotide sequence having at least 95% identity with the amino acid sequence encoded by nucleotides 18528-19508 of SEQ ID NO:199 (herein termed GT5);
(iii) (a): a nucleotide sequence encoding a polypeptide having polymerase activity and having at least 95% identity with the amino acid sequence encoded by the nucleotide sequence of SEQ ID NO: 39 (herein termed wzy);
(b): a nucleotide sequence encoding a polypeptide having polysaccharide transporter activity and having at least 95% identity with the amino acid sequence encoded by the nucleotide sequence of SEQ ID NO: 45 (herein termed wzx);
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 37 (herein termed GT1);
(c2): a nucleotide sequence having at least 95% identity with the amino acid sequence encoded by the nucleotide sequence of SEQ ID NO: 41 (herein termed GT2); and
(c3): a nucleotide sequence having at least 95% identity with the amino acid sequence encoded by the nucleotide sequence of SEQ ID NO: 43 (herein termed GT3);
(iv) (a): a nucleotide sequence encoding a polypeptide having polymerase activity and having at least 95% identity with the amino acid sequence encoded by the nucleotide sequence of SEQ ID NO: 163 (herein termed wzy);
(b): a nucleotide sequence encoding a polypeptide having polysaccharide transporter activity and having at least 95% identity with the amino acid sequence encoded by the nucleotide sequence of SEQ ID NO: 169 (herein termed wzx); and
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 161 (herein termed GT1);
(c2): a nucleotide sequence having at least 95% identity with the amino acid sequence encoded by the nucleotide sequence of SEQ ID NO: 165 (herein termed GT2);
(c3): a nucleotide sequence having at least 95% identity with the amino acid sequence encoded by the nucleotide sequence of SEQ ID NO: 167 (herein termed GT3); and
(c4) a nucleotide sequence having at least 95% identity with the amino acid sequence encoded by the nucleotide sequence of SEQ ID NO: 181 (herein termed GT4);
(v) (a): a nucleotide sequence encoding a polypeptide having polymerase activity and having at least 95% identity with the amino acid sequence encoded by nucleotides 13939-15042 of SEQ ID NO: 224 (herein termed wzy); and
(c): a nucleotide sequence encoding a polypeptide having glycosyltransferase (GT) activity comprising:
(c1): a nucleotide sequence having at least 95% identity with the amino acid sequence encoded by nucleotides 4174-4824 of SEQ ID NO: 224 (herein termed GT1);
(c2): a nucleotide sequence having at least 95% identity with the amino acid sequence encoded by nucleotides 11042-12391 of SEQ ID NO: 224 (herein termed GT2);
(c3): a nucleotide sequence having at least 95% identity with the amino acid sequence encoded by nucleotides 13008-13934 of SEQ ID NO: 224 (herein termed GT3); and
(c4): a nucleotide sequence having at least 95% identity with the amino acid sequence encoded by nucleotides 18527-19507 of SEQ ID NO: 224 (herein termed GT4);
(vi) (a): a nucleotide sequence encoding a polypeptide having polymerase activity and having at least 95% identity with the amino acid sequence encoded by the nucleotide sequence of SEQ ID NO: 67 (herein termed wzy);
(b): a nucleotide sequence encoding a polypeptide having polysaccharide transporter activity and having at least 95% identity with the amino acid sequence encoded by the nucleotide sequence of SEQ ID NO: 73 (herein termed wzx);
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 65 (herein termed GT1);
(c2): a nucleotide sequence having at least 95% identity with the amino acid sequence encoded by the nucleotide sequence of SEQ ID NO: 69 (herein termed GT2); and
(c3): a nucleotide sequence having at least 95% identity with the amino acid sequence encoded by the nucleotide sequence of SEQ ID NO: 71 (herein termed GT3);
(c4): a nucleotide sequence having at least 95% identity with the amino acid sequence encoded by the nucleotide sequence of SEQ ID NO: 85 (herein termed GT4);
(c5): a nucleotide sequence having at least 95% identity with the amino acid sequence encoded by the nucleotide sequence of SEQ ID NO: 87 (herein termed GT5); and
(c6): a nucleotide sequence having at least 95% identity with the amino acid sequence encoded by the nucleotide sequence of SEQ ID NO: 89 (herein termed GT6);
(vii) (a): a nucleotide sequence encoding a polypeptide having polymerase activity and having at least 95% identity with the amino acid sequence encoded by nucleotides 5833-6927 of SEQ ID NO: 244 (herein termed wzy);
and
(c): a nucleotide sequence encoding a polypeptide having glycosyltransferase (GT) activity comprising:
(c1): a nucleotide sequence having at least 95% identity with the amino acid sequence encoded by nucleotides 4617-5123 of SEQ ID NO: 244 (herein termed GT1); and
(c2): a nucleotide sequence having at least 95% identity with the amino acid sequence encoded by nucleotides 5120-5827 of SEQ ID NO: 244 (herein termed GT2);
(viii) (a): a nucleotide sequence encoding a polypeptide having polymerase activity and having at least 95% identity with the amino acid sequence encoded by the nucleotide sequence of SEQ ID NO: 123 (herein termed wzy);
(b): a nucleotide sequence encoding a polypeptide having polysaccharide transporter activity and having at least 95% identity with the amino acid sequence encoded by the nucleotide sequence of SEQ ID NO: 129 (herein termed wzx);
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 121 (herein termed GT1);
(c2): a nucleotide sequence having at least 95% identity with the amino acid sequence encoded by the nucleotide sequence of SEQ ID NO: 125 (herein termed GT2); and
(c3): a nucleotide sequence having at least 95% identity with the amino acid sequence encoded by the nucleotide sequence of SEQ ID NO: 127 (herein termed GT3);
(c4): a nucleotide sequence having at least 95% identity with the amino acid sequence encoded by the nucleotide sequence of SEQ ID NO: 143 (herein termed GT4);
(c5): a nucleotide sequence having at least 95% identity with the amino acid sequence encoded by the nucleotide sequence of SEQ ID NO: 145 (herein termed GT5); and
(c6): a nucleotide sequence having at least 95% identity with the amino acid sequence encoded by the nucleotide sequence of SEQ ID NO: 147 (herein termed GT6);
(ix) (a): a nucleotide sequence encoding a polypeptide having polymerase activity and having at least 95% identity with the amino acid sequence encoded by nucleotides 11201-12349 of the complementary strand of SEQ ID NO: 257 (herein termed wzy);
(b): a nucleotide sequence encoding a polypeptide having polysaccharide transporter activity and having at least 95% identity with the amino acid sequence encoded by nucleotides 15538-16953 of the complementary strand of SEQ ID NO: 257 (herein termed wzx); and
(c): a nucleotide sequence encoding a polypeptide having glycosyltransferase (GT) activity comprising:
(c1): a nucleotide sequence having at least 95% identity with the amino acid sequence encoded by nucleotides 9726-10673 of the complementary strand of SEQ ID NO: 257 (herein termed GT1);
(c2): a nucleotide sequence having at least 95% identity with the amino acid sequence encoded by nucleotides 12336-13421 of the complementary strand of SEQ ID NO: 257 (herein termed GT2); and
(c3): a nucleotide sequence having at least 95% identity with the amino acid sequence encoded by nucleotides 13418-14260 of the complementary strand of SEQ ID NO: 257 (herein termed GT3);
(x) (a): a nucleotide sequence encoding a polypeptide having polymerase activity and having at least 95% identity with the amino acid sequence encoded by nucleotides 10707-11846 of the complementary strand of SEQ ID NO: 274 (herein termed wzy);
(b): a nucleotide sequence encoding a polypeptide having polysaccharide transporter activity and having at least 95% identity with the amino acid sequence encoded by nucleotides 15037-16476 of the complementary strand of SEQ ID NO: 274 (herein termed wzx); and
(c): a nucleotide sequence encoding a polypeptide having glycosyltransferase (GT) activity comprising:
(c1): a nucleotide sequence having at least 95% identity with the amino acid sequence encoded by nucleotides 9232-10179 of the complementary strand of SEQ ID NO: 274 (herein termed GT1);
(c2): a nucleotide sequence having at least 95% identity with the amino acid sequence encoded by nucleotides 11833-12918 of the complementary strand of SEQ ID NO: 274 (herein termed GT2); and
(c3): a nucleotide sequence having at least 95% identity with the amino acid sequence encoded by nucleotides 12915-13757 of the complementary strand of SEQ ID NO: 274 (herein termed GT3).

2. The Lactococcus lactis lactic acid bacterium strain according to claim 1, wherein the eps gene cluster comprises nucleotide sequences selected from (i) to (x):
(i) (a): a nucleotide sequence encoding a polypeptide having polymerase activity and having at least 95% identity with the amino acid sequence encoded by the nucleotide sequence of SEQ ID NO: 11 (herein termed wzy);
(b): a nucleotide sequence encoding a polypeptide having polysaccharide transporter activity and having at least 95% identity with the amino acid sequence encoded by the nucleotide sequence of SEQ ID NO: 17 (herein termed wzx);
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 9 (herein termed GT1);
(c2): a nucleotide sequence having at least 95% identity with the amino acid sequence encoded by the nucleotide sequence of SEQ ID NO: 13 (herein termed GT2); and
(c3): a nucleotide sequence having at least 95% identity with the amino acid sequence encoded by the nucleotide sequence of SEQ ID NO: 15 (herein termed GT3); and
(d): a nucleotide sequence having at least 95%, identity with the amino acid sequence encoded by the nucleotide sequence of SEQ ID NO: 23 (herein termed putative nucleotide sugar dehydrogenase protein);
(ii) (a): a nucleotide sequence encoding a polypeptide having polymerase activity and having at least 95% identity with the amino acid sequence encoded by nucleotides 13939-15042 of SEQ ID NO:199 (herein termed wzy);
(b): a nucleotide sequence encoding a polypeptide having polysaccharide transporter activity and having at least 95% identity with the amino acid sequence encoded by nucleotides 26029-27444 of the complementary strand of SEQ ID NO:199 (herein termed wzx);
(c): at least one nucleotide sequence encoding a polypeptide having glycosyltransferase (GT) activity selected from:
(c1): a nucleotide sequence having at least 95% identity with the amino acid sequence encoded by nucleotides 4174-4824 of SEQ ID NO:199 (herein termed GT1);
(c2): a nucleotide sequence having at least 95% identity with the amino acid sequence encoded by nucleotides 7276-8508 of SEQ ID NO:199 (herein termed GT2);
(c3): a nucleotide sequence having at least 95% identity with the amino acid sequence encoded by nucleotides 11042-12391 of SEQ ID NO:199 (herein termed GT3);
(c4): a nucleotide sequence having at least 95% identity with the amino acid sequence encoded by nucleotides 13008-13934 of SEQ ID NO:199 (herein termed GT4);
(c5): a nucleotide sequence having at least 95% identity with the amino acid sequence encoded by nucleotides 18528-19508 of SEQ ID NO:199 (herein termed GT5);
(d): a nucleotide sequence encoding a polypeptide having dTDP-glucose 4,6-dehydratase activity and having at least 95% identity with the amino acid sequence encoded by nucleotides 4784-5695 of SEQ ID NO:199 (herein termed dTDP-glucose 4,6-dehydratase);
(e): a nucleotide sequence encoding a polypeptide having dTDP-4-dehydrorhamnose reductase activity and having at least 95% identity with the amino acid sequence encoded by nucleotides 5717-6631 of SEQ ID NO:199 (herein termed dTDP-4-dehydrorhamnose reductase);
(f): a nucleotide sequence encoding a polypeptide having dTDP-4-dehydrorhamnose 3,5-epimerase activity and having at least 95% identity with the amino acid sequence encoded by nucleotides 6586-7257 of SEQ ID NO:199 (herein termed dTDP-4-dehydrorhamnose 3,5-epimerase);
(g): a nucleotide sequence encoding polypeptide DUF1919 and having at least 95% identity with the amino acid sequence encoded by nucleotides 8515-9144 of SEQ ID NO:199 (herein termed DUF1919);
(h): a nucleotide sequence encoding polypeptide DUF4422 and having at least 95% identity with the amino acid sequence encoded by nucleotides 10271-11029 of SEQ ID NO:199 (herein termed DUF4422); and
(i): a nucleotide sequence encoding a polypeptide having UDP-galactopyranose mutase activity and having at least 95% identity with the amino acid sequence encoded by nucleotides 9159-10274 of SEQ ID NO:199 (herein termed UDP-galactopyranose mutase);
(iii) (a): a nucleotide sequence encoding a polypeptide having polymerase activity and having at least 95% identity with the amino acid sequence encoded by the nucleotide sequence of SEQ ID NO: 39 (herein termed wzy);
(b): a nucleotide sequence encoding a polypeptide having polysaccharide transporter activity and having at least 95% identity with the amino acid sequence encoded by the nucleotide sequence of SEQ ID NO: 45 (herein termed wzx);
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 37 (herein termed GT1);
(c2): a nucleotide sequence having at least 95% identity with the amino acid sequence encoded by the nucleotide sequence of SEQ ID NO: 41 (herein termed GT2); and
(c3): a nucleotide sequence having at least 95% identity with the amino acid sequence encoded by the nucleotide sequence of SEQ ID NO: 43 (herein termed GT3); and
(d): a nucleotide sequence having polysaccharide pyruvyl transferase activity and having at least 95% identity with the amino acid sequence encoded by the nucleotide sequence of SEQ ID NO: 51 (herein termed polysaccharide pyruvyl transferase family protein);
(iv) (a): a nucleotide sequence encoding a polypeptide having polymerase activity and having at least 95% identity with the amino acid sequence encoded by the nucleotide sequence of SEQ ID NO: 163 (herein termed wzy);
(b): a nucleotide sequence encoding a polypeptide having polysaccharide transporter activity and having at least 95% identity with the amino acid sequence encoded by the nucleotide sequence of SEQ ID NO: 169 (herein termed wzx);
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 161 (herein termed GT1);
(c2): a nucleotide sequence having at least 95% identity with the amino acid sequence encoded by the nucleotide sequence of SEQ ID NO: 165 (herein termed GT2);
(c3): a nucleotide sequence having at least 95% identity with the amino acid sequence encoded by the nucleotide sequence of SEQ ID NO: 167 (herein termed GT3); and
(c4) a nucleotide sequence having at least 95% identity with the amino acid sequence encoded by the nucleotide sequence of SEQ ID NO: 181 (herein termed GT4); and
(d) a nucleotide sequence encoding a polypeptide having at least 95% identity with the amino acid sequence encoded by the nucleotide sequence of SEQ ID NO: 175 (herein termed Core-2/I-Branching protein);
(v) (a): a nucleotide sequence encoding a polypeptide having polymerase activity and having at least 95% identity with the amino acid sequence encoded by nucleotides 13939-15042 of SEQ ID NO: 224 (herein termed wzy);
(c): at least one nucleotide sequence encoding a polypeptide having glycosyltransferase (GT) activity selected from:
(c1): a nucleotide sequence having at least 95% identity with the amino acid sequence encoded by nucleotides 4174-4824 of SEQ ID NO: 224 (herein termed GT1);
(c2): a nucleotide sequence having at least 95% identity with the amino acid sequence encoded by nucleotides 11042-12391 of SEQ ID NO: 224 (herein termed GT2);
(c3): a nucleotide sequence having at least 95% identity with the amino acid sequence encoded by nucleotides 13008-13934 of SEQ ID NO: 224 (herein termed GT3); and
(c4): a nucleotide sequence having at least 95% identity with the amino acid sequence encoded by nucleotides 18527-19507 of SEQ ID NO: 224 (herein termed GT4);
(d): a nucleotide sequence encoding polypeptide DUF1972, and having at least 95% identity with the amino acid sequence encoded by nucleotides 7276-8508 of SEQ ID NO: 224 (herein termed DUF1972);
(e): a nucleotide sequence encoding polypeptide DUF4422 and having at least 95% identity with the amino acid sequence encoded by nucleotides 10271-11029 of SEQ ID NO: 224 (herein termed DUF4422);
(f): a nucleotide sequence encoding polypeptide DUF1919 and having at least 95% identity with the amino acid sequence encoded by nucleotides 8515-9144 of SEQ ID NO: 224 (herein termed DUF1919);
(g): a nucleotide sequence encoding a polypeptide having UDP-galactopyranose mutase activity and having at least 95% identity with the amino acid sequence encoded by nucleotides 9159-10274 of SEQ ID NO: 224 (herein termed UDP-galactopyranose mutase);
(h): a nucleotide sequence encoding a polypeptide having dTDP-4-dehydrorhamnose_3,5-epimerase activity and having at least 95% identity with the amino acid sequence encoded by nucleotides 6586-7257 of SEQ ID NO: 224 (herein termed dTDP-4-dehydrorhamnose_3,5-epimerase);
(i): a nucleotide sequence encoding a polypeptide having dTDP-glucose 4,6-dehydratase activity and having at least 95% identity with the amino acid sequence encoded by nucleotides 4784-5695 of SEQ ID NO: 224 (herein termed dTDP-glucose 4,6-dehydratase); and
(j): a nucleotide sequence encoding a polypeptide having dTDP-4-dehydrorhamnose reductase activity and having at least 95% identity with the amino acid sequence encoded by nucleotides 5717-6631 of SEQ ID NO: 224 (herein termed dTDP-4-dehydrorhamnose reductase);
(vi) (a): a nucleotide sequence encoding a polypeptide having polymerase activity and having at least 95% identity with the amino acid sequence encoded by the nucleotide sequence of SEQ ID NO: 67 (herein termed wzy);
(b): a nucleotide sequence encoding a polypeptide having polysaccharide transporter activity and having at least 95% identity with the amino acid sequence encoded by the nucleotide sequence of SEQ ID NO: 73 (herein termed wzx);
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 65 (herein termed GT1);
(c2): a nucleotide sequence having at least 95% identity with the amino acid sequence encoded by the nucleotide sequence of SEQ ID NO: 69 (herein termed GT2); and
(c3): a nucleotide sequence having at least 95% identity with the amino acid sequence encoded by the nucleotide sequence of SEQ ID NO: 71 (herein termed GT3);
(c4): a nucleotide sequence having at least 95% identity with the amino acid sequence encoded by the nucleotide sequence of SEQ ID NO: 85 (herein termed GT4);
(c5): a nucleotide sequence having at least 95% identity with the amino acid sequence encoded by the nucleotide sequence of SEQ ID NO: 87 (herein termed GT5); and
(c6): a nucleotide sequence having at least 95% identity with the amino acid sequence encoded by the nucleotide sequence of SEQ ID NO: 89 (herein termed GT6);
(d): a nucleotide sequence having epimerase/dehydratase activity and having at least 95% identity with the amino acid sequence encoded by the nucleotide sequence of SEQ ID NO: 93 (herein termed NAD-dependent epimerase/dehydratase 1);
(e): a nucleotide sequence having dehydrogenase activity and having at least 95% identity with the amino acid sequence encoded by the nucleotide sequence of SEQ ID NO: 79 (herein termed nucleotide sugar dehydrogenase);
(f): a nucleotide sequence having thymidylyltransferase activity and having at least 95% identity with the amino acid sequence encoded by the nucleotide sequence of SEQ ID NO: 95 (herein termed rfbA, glucose-1-phosphate thymidylyltransferase);
(g): a nucleotide sequence having dehydratase activity and having at least 95% identity with the amino acid sequence encoded by the nucleotide sequence of SEQ ID NO: 97 (herein termed dTDP-glucose 4,6-dehydratase);
(h): a nucleotide sequence having epimerase activity and having at least 95% identity with the amino acid sequence encoded by the nucleotide sequence of SEQ ID NO: 99 (herein termed dTDP-4-dehydrorhamnose 3,5-epimerase);
(i): a nucleotide sequence having epimerase/dehydratase activity and having at least 95% identity with the amino acid sequence encoded by the nucleotide sequence of SEQ ID NO: 101 (herein termed NAD-dependent epimerase/dehydratase family protein 2);
(j): a nucleotide sequence having acyltransferase activity and having at least 95% identity with the amino acid sequence encoded by the nucleotide sequence of SEQ ID NO: 111 (herein termed acyltransferase 1);
(k): a nucleotide sequence having acyltransferase activity and having at least 95% identity with the amino acid sequence encoded by the nucleotide sequence of SEQ ID NO: 107 (herein termed acyltransferase 2);
(l): a nucleotide sequence having reductase activity and having at least 95% identity with the amino acid sequence encoded by the nucleotide sequence of SEQ ID NO: 103 (herein termed dTDP-4-dehydrorhamnose reductase); and
(m): a nucleotide sequence having nucleotidyl transferase activity and having at least 95% identity with the amino acid sequence encoded by the nucleotide sequence of SEQ ID NO: 105 (herein termed nucleotidyl transferase).
(vii) (a): a nucleotide sequence encoding a polypeptide having polymerase activity and having at least 95% identity with the amino acid sequence encoded by nucleotides 5833-6927 of SEQ ID NO: 244 (herein termed wzy);
(c): at least one nucleotide sequence encoding a polypeptide having glycosyltransferase (GT) activity selected from:
(c1): a nucleotide sequence having at least 95% identity with the amino acid sequence encoded by nucleotides 4617-5123 of SEQ ID NO: 244 (herein termed GT1); and
(c2): a nucleotide sequence having at least 95% identity with the amino acid sequence encoded by nucleotides 5120-5827 of SEQ ID NO: 244 (herein termed GT2); and
(d): a nucleotide sequence encoding a polypeptide having UDP-N-acetylglucosamine-LPS N-acetylglucosamine transferase activity and having at least 95% identity with the amino acid sequence encoded by nucleotides 4168-4617 of SEQ ID NO: 244 (herein termed UDP-N-acetylglucosamine-LPS N-acetylglucosamine transferase);
(viii) (a): a nucleotide sequence encoding a polypeptide having polymerase activity and having at least 95% identity with the amino acid sequence encoded by the nucleotide sequence of SEQ ID NO: 123 (herein termed wzy);
(b): a nucleotide sequence encoding a polypeptide having polysaccharide transporter activity and having at least 95% identity with the amino acid sequence encoded by the nucleotide sequence of SEQ ID NO: 129 (herein termed wzx);
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 121 (herein termed GT1);
(c2): a nucleotide sequence having at least 95% identity with the amino acid sequence encoded by the nucleotide sequence of SEQ ID NO: 125 (herein termed GT2);
(c3): a nucleotide sequence having at least 95% identity with the amino acid sequence encoded by the nucleotide sequence of SEQ ID NO: 127 (herein termed GT3);
(c4): a nucleotide sequence having at least 95% identity with the amino acid sequence encoded by the nucleotide sequence of SEQ ID NO: 143 (herein termed GT4);
(c5): a nucleotide sequence having at least 95% identity with the amino acid sequence encoded by the nucleotide sequence of SEQ ID NO: 145 (herein termed GT5); and
(c6): a nucleotide sequence having at least 95% identity with the amino acid sequence encoded by the nucleotide sequence of SEQ ID NO: 147 (herein termed GT6);
(d): a nucleotide sequence encoding a polypeptide having acetyltransferase activity and having at least 95% identity with the amino acid sequence encoded by the nucleotide sequence of SEQ ID NO: 149 (herein termed acetyltransferase);
(e): a nucleotide sequence encoding a polypeptide having dehydrogenase activity and having at least 95% identity with the amino acid sequence encoded by the nucleotide sequence of SEQ ID NO: 135 (herein termed nucleotide sugar dehydrogenase); and
(f): a nucleotide sequence encoding a polypeptide having acyltransferase activity and having at least 95% identity with the amino acid sequence encoded by the nucleotide sequence of SEQ ID NO: 151 (herein termed acyltransferase);
(ix) (a): a nucleotide sequence encoding a polypeptide having polymerase activity and having at least 95% identity with the amino acid sequence encoded by nucleotides 11201-12349 of the complementary strand of SEQ ID NO: 257 (herein termed wzy);
(b): a nucleotide sequence encoding a polypeptide having polysaccharide transporter activity and having at least 95% identity with the amino acid sequence encoded by nucleotides 15538-16953 of the complementary strand of SEQ ID NO: 257 (herein termed wzx);
(c): a nucleotide sequence encoding a polypeptide having glycosyltransferase (GT) activity comprising:
(c1): a nucleotide sequence having at least 95% identity with the amino acid sequence encoded by nucleotides 9726-10673 of the complementary strand of SEQ ID NO: 257 (herein termed GT1);
(c2): a nucleotide sequence having at least 95% identity with the amino acid sequence encoded by nucleotides 12336-13421 of the complementary strand of SEQ ID NO: 257 (herein termed GT2); and
(c3): a nucleotide sequence having at least 95% identity with the amino acid sequence encoded by nucleotides 13418-14260 of the complementary strand of SEQ ID NO: 257 (herein termed GT3);
(d): a nucleotide sequence encoding a polypeptide having nucleotide sugar dehydrogenase activity and having at least 95% identity with the amino acid sequence encoded by nucleotides 7727-8173 of the complementary strand of SEQ ID NO: 257 (herein termed nucleotide sugar dehydrogenase); and
(e): a nucleotide sequence encoding a polypeptide having acetyltransferase activity and having at least 95% identity with the amino acid sequence encoded by nucleotides 10657-11211 of the complementary strand of SEQ ID NO: 257 (herein termed acetyltransferase);
(x) (a): a nucleotide sequence encoding a polypeptide having polymerase activity and having at least 95% identity with the amino acid sequence encoded by nucleotides 10707-11846 of the complementary strand of SEQ ID NO: 274 (herein termed wzy);
(b): a nucleotide sequence encoding a polypeptide having polysaccharide transporter activity and having at least 95% identity with the amino acid sequence encoded by nucleotides 15037-16476 of the complementary strand of SEQ ID NO: 274 (herein termed wzx);
(c): a nucleotide sequence encoding a polypeptide having glycosyltransferase (GT) activity comprising:
(c1): a nucleotide sequence having at least 95% identity with the amino acid sequence encoded by nucleotides 9232-10179 of the complementary strand of SEQ ID NO: 274 (herein termed GT1);
(c2): a nucleotide sequence having at least 95% identity with the amino acid sequence encoded by nucleotides 11833-12918 of the complementary strand of SEQ ID NO: 274 (herein termed GT2); and
(c3): a nucleotide sequence having at least 95% identity with the amino acid sequence encoded by nucleotides 12915-13757 of the complementary strand of SEQ ID NO: 274 (herein termed GT3);
(d): a nucleotide sequence encoding a polypeptide having nucleotide sugar dehydrogenase activity and having at least 95% identity with the amino acid sequence encoded by nucleotides 7234-7680 of the complementary strand of SEQ ID NO: 274 (herein termed nucleotide sugar dehydrogenase); and
(e): a nucleotide sequence encoding a polypeptide having acetyltransferase activity and having at least 95% identity with the amino acid sequence encoded by nucleotides 10163-10717 of the complementary strand of SEQ ID NO: 274 (herein termed acetyltransferase).

3. The texturing lactic acid bacterium strain of clause (iv), clause (vii), clause (ix) or clause (x) of claim 1, wherein the texturing lactic acid bacterium strain generates fermented milks having a shear stress of 45 Pa or more when measured under the following conditions:
200 ml semi-fat milk (1.5% fat) is heated to 90° C. for 20 min, followed by cooling to an inoculation temperature of 30° C., and inoculated with 2 ml of an overnight culture of the lactic acid bacterium strain, and left at inoculation temperature until a pH of about 4.55 is reached, followed by storage at 4° C. until shear stress is measured at day 5, followed by gently stirring and measuring the shear stress at a shear rate of 300 s⁻¹.

4. The texturing lactic acid bacterium strain according to claim 3, wherein the strain is selected from the group consisting of strains deposited at Leibniz Institute DSMZ-German Collection of Microorganisms and Cell Cultures Inhoffenstr (DSMZ) under accession numbers DSM 33137, DSM 33140, DSM 33142 and DSM 33183, and strains derived from deposited strains DSM 33137, DSM 33140, DSM 33142 or DSM 33183, wherein the derived strains exhibit at least the same texturing capability as their respective mother strain selected from deposited strains DSM 33137, DSM 33140, DSM 33142 and DSM 33183, and generate fermented milks having a shear stress of 45 Pa or more when measured under the following conditions:
200 ml semi-fat milk (1.5% fat) is heated to 90° C. for 20 min, followed by cooling to an inoculation temperature of 30° C., and inoculated with 2 ml of an overnight culture of the lactic acid bacterium strain, and left at inoculation temperature until a pH of about 4.55 is reached, followed by storage at 4° C. until shear stress is measured at day 5, followed by gently stirring and measuring the shear stress at a shear rate of 300 s⁻¹.

5. The texturing lactic acid bacterium strain of claim 1, wherein the texturing lactic acid bacterium strain generates fermented milks having a shear stress of 40 Pa or more when measured at shear rate 300 s⁻¹ in the presence of a lactic acid bacterium strain selected from Lactococcus lactis subsp. cremoris strain deposited at DSMZ under accession number DSM 25485 and Lactococcus lactis subsp. lactis strain deposited at DSMZ under accession number DSM 33192, in a ratio of about 9:1 (texturing lactic acid bacterium strain: strain DSM 25485 and/or or DSM 33192), wherein the shear stress is measured under following conditions:
200 ml semi-fat milk (1.5% fat) is heated to 90° C. for 20 min, followed by cooling to an inoculation temperature of 30° C., and inoculated with 2 ml of an overnight culture of the lactic acid bacterium strains, and left at inoculation temperature until a pH of about 4.55 is reached, followed by storage at 4° C. until shear stress is measured at day 5, followed by gently stirring and measuring the shear stress at a shear rate of 300 s⁻¹.

6. The texturing lactic acid bacterium strain of clause (i), clause (ii), clause (iii), clause (v), clause (vi) or clause (viii) of claim 1, wherein the strain is selected from the strains deposited at DSMZ under accession numbers DSM 33134, DSM 33135, DSM 33136, DSM 33138, DSM 33139, and DSM 33141, and strains derived from deposited strains DSM 33139, DSM 33135, DSM 33136, DSM 33138, DSM 33139, or DSM 33141, wherein the derived strains exhibit at least the same texturing capability as their respective mother strain selected from deposited strains DSM 33134, DSM 33135, DSM 33136, DSM 33138, DSM 33139, and DSM 33141, and generate fermented milks having a shear stress of 40 Pa or more when measured at shear rate 300 s⁻¹ in the presence of a lactic acid bacterium strain selected from Lactococcus lactis subsp. cremoris strain deposited at DSMZ under accession number DSM 25485, and Lactococcus lactis subsp. lactis strain deposited at DSMZ under accession number DSM 33192, in a ratio of about 9:1 (texturing lactic acid bacterium strain: strain DSM 25485 or DSM 33192), wherein the shear stress is measured under following conditions:
200 ml semi-fat milk (1.5% fat) is heated to 90° C. for 20 min, followed by cooling to an inoculation temperature of 30° C., and inoculated with 2 ml of an overnight culture of the lactic acid bacterium strains, and left at inoculation temperature until a pH of about 4.55 is reached, followed by storage at 4° C. until shear stress is measured at day 5, followed by gently stirring and measuring the shear stress at a shear rate of 300 s⁻¹.

7. The texturing lactic acid bacterium strain of claim 1, wherein the texturing lactic acid bacterium strain generates fermented milks having a shear stress of more than 24 Pa when measured under the following conditions:
200 ml of soy milk supplemented with 2% glucose is inoculated with 2 ml of an overnight culture of the lactic acid bacterium strain, and left at an inoculation temperature of 30° C. until a pH of about 4.56 is reached, followed by storage at 4° C. until shear stress is measured at day 5, followed by gently stirring and measuring the shear stress at a shear rate of 300 s⁻¹.

8. The texturing lactic acid bacterium strain of claim 7, wherein the texturing lactic acid bacterium strain is selected from the strains deposited at DSMZ under accession numbers DSM 33138, DSM 33140, DSM 33136, DSM 33135, DSM 33141, DSM 33137, DSM 33134, DSM 33139, DSM 33142, DSM 33192, DSM 25485, and DSM 33183, and strains derived from strains DSM 33138, DSM 33140, DSM 33136, DSM 33135, DSM 33141, DSM 33137, DSM 33134, DSM 33139, DSM 33142, DSM 33192, DSM 25485, or DSM 33183, wherein the derived strains exhibit at least the same texturing capability as their respective mother strain selected from deposited strains DSM 33138, DSM 33140, DSM 33136, DSM 33135, DSM 33141, DSM 33137, DSM 33134, DSM 33139, DSM 33142, DSM 33192, DSM 25485, and DSM 33183, and generate fermented milks having a shear stress of more than 24 Pa, when measured under the following conditions:
200 ml of soy milk supplemented with 2% is inoculated with 2 ml of an overnight culture of the lactic acid bacterium strain, and left at an inoculation temperature of 30° C. until a pH of about 4.56 is reached, followed by storage at 4° C. until shear stress is measured at day 5, followed by gently stirring and measuring the shear stress at a shear rate of 300 s⁻¹.

9. A composition comprising the Lactococcus lactis lactic acid bacterium strain of claim 1 and one or more further lactic acid bacterium strain(s), wherein the one or more further lactic acid bacterium strain(s) is(are) able to:
i) generate fermented milks with a pH of about 4.55 in 15 hours or less under the following conditions:
200 ml semi-fat milk (1.5% fat) is heated to 90° C. for 20 min, followed by cooling to an inoculation temperature of 30° C., and inoculated with 2 ml of an overnight culture of the lactic acid bacterium strain, and left at inoculation temperature until a pH of about 4.55 is reached; and
ii) generate fermented milks having a shear stress of 40 Pa or more measured under the following conditions:
200 ml semi-fat milk (1.5% fat) is heated to 90° C. for 20 min, followed by cooling to an inoculation temperature of 30° C., and inoculated with 2 ml of an overnight culture of the lactic acid bacterium strain, and left at inoculation temperature until a pH of about 4.55 is reached, followed by storage at 4° C. until shear stress is measured at day 5, followed by gently stirring and measuring the shear stress at a shear rate of 300 s⁻¹.

10. The composition according to claim 9, wherein the one or more further lactic acid bacterium strain(s) comprises an active eps gene cluster capable of producing exopolysaccharide (EPS), wherein the eps gene cluster comprises:
(xi) (a): a nucleotide sequence encoding a polypeptide having polymerase activity and having at least 95% identity with the amino acid sequence encoded by nucleotides 6955-8145 of SEQ ID NO:183 (herein termed wzy);
(b): a nucleotide sequence encoding a polypeptide having polysaccharide transporter activity and having at least 95% identity with the amino acid sequence encoded by nucleotides 9309-10727 of SEQ ID NO:183 (herein termed wzx); and
(c): a nucleotide sequence encoding a polypeptide having glycosyltransferase (GT) activity comprising:
(c1): a nucleotide sequence having at least 95% identity with the amino acid sequence encoded by nucleotides 4008-4478 of SEQ ID NO:183 (herein termed GT1);
(c2): a nucleotide sequence having at least 95% identity with the amino acid sequence encoded by nucleotides 4478-4960 of SEQ ID NO:183 (herein termed GT2);
(c3): a nucleotide sequence having at least 95% identity with the amino acid sequence encoded by nucleotides 5015-5965 of SEQ ID NO:183 (herein termed GT3); and
(c4): a nucleotide sequence having at least 95% identity with the amino acid sequence encoded by nucleotides 6026-6955 of SEQ ID NO:183 (herein termed GT4).

11. The composition according to claim 9, further comprising one or more lactic acid bacterium strains selected from:
(i) Lactococcus lactis sub sp. cremoris strain DSM 25485, or a mutant or variant thereof;
(ii) Lactococcus lactis subsp. lactis strain DSM 33192, or a mutant or variant thereof; and
(iii) Lactococcus lactis strain DSM 33133, or a mutant or variant thereof.

12. (canceled)

13. A method of producing a food product comprising fermenting a milk substrate with at least one lactic acid bacterium strain according to claim 1.

14. The method according to claim 13, wherein the method comprises fermenting the milk substrate with a composition according to claim 9.

15. A food product comprising at least one lactic acid bacterium strain according to claim 1.

16. (canceled)

17. The method according to claim 21, wherein the fermented milk product has a shear stress of 50 Pa or more when measured under following conditions:
200 ml semi-fat milk (1.5% fat) is heated to 90° C. for 20 min, followed by cooling to an inoculation temperature of 30° C., and inoculated with 2 ml of an overnight culture of the lactic acid bacterium strain, and left at inoculation temperature until a pH of about 4.55 is reached, followed by storage at 4° C. until shear stress is measured at day 5, followed by gently stirring and measuring the shear stress at a shear rate of 300 s⁻¹.

18. The method according to claim 21, further comprising fermenting the milk substrate with at least one Lactococcus lactis lactic acid bacterium strain comprising an active eps gene cluster capable of producing exopolysaccharide (EPS), wherein the eps gene cluster comprises nucleotide sequences selected from (i) to (x):
(i) (a): a nucleotide sequence encoding a polypeptide having polymerase activity and having at least 95% identity with the amino acid sequence encoded by the nucleotide sequence of SEQ ID NO: 11 (herein termed wzy);
(b): a nucleotide sequence encoding a polypeptide having polysaccharide transporter activity and having at least 95% identity with the amino acid sequence encoded by the nucleotide sequence of SEQ ID NO: 17 (herein termed wzx); and
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 9 (herein termed GT1),
(c2): a nucleotide sequence having at least 95% identity with the amino acid sequence encoded by the nucleotide sequence of SEQ ID NO: 13 (herein termed GT2), and
(c3): a nucleotide sequence having at least 95% identity with the amino acid sequence encoded by the nucleotide sequence of SEQ ID NO: 15 (herein termed GT3);
(ii) (a): a nucleotide sequence encoding a polypeptide having polymerase activity and having at least 95% identity with the amino acid sequence encoded by nucleotides 13939-15042 of SEQ ID NO:199 (herein termed wzy);
(b): a nucleotide sequence encoding a polypeptide having polysaccharide transporter activity and having at least 95% identity with the amino acid sequence encoded by nucleotides 26029-27444 of the complementary strand of SEQ ID NO:199 (herein termed wzx); and
(c): a nucleotide sequence encoding a polypeptide having glycosyltransferase (GT) activity comprising:
(c1): a nucleotide sequence having at least 95% identity with the amino acid sequence encoded by nucleotides 4174-4824 of SEQ ID NO:199 (herein termed GT1);
(c2): a nucleotide sequence having at least 95% identity with the amino acid sequence encoded by nucleotides 7276-8508 of SEQ ID NO:199(herein termed GT2);
(c3): a nucleotide sequence having at least 95% identity with the amino acid sequence encoded by nucleotides 11042-12391 of SEQ ID NO:199 (herein termed GT3);
(c4): a nucleotide sequence having at least 95% identity with the amino acid sequence encoded by nucleotides 13008-13934 of SEQ ID NO:199 (herein termed GT4); and
(c5): a nucleotide sequence having at least 95% identity with the amino acid sequence encoded by nucleotides 18528-19508 of SEQ ID NO:199 (herein termed GT5);
(iii) (a): a nucleotide sequence encoding a polypeptide having polymerase activity and having at least 95% identity with the amino acid sequence encoded by the nucleotide sequence of SEQ ID NO: 39 (herein termed wzy);
(b): a nucleotide sequence encoding a polypeptide having polysaccharide transporter activity and having at least 95% identity with the amino acid sequence encoded by the nucleotide sequence of SEQ ID NO: 45 (herein termed wzx);
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 37 (herein termed GT1);
(c2): a nucleotide sequence having at least 95% identity with the amino acid sequence encoded by the nucleotide sequence of SEQ ID NO: 41 (herein termed GT2); and
(c3): a nucleotide sequence having at least 95% identity with the amino acid sequence encoded by the nucleotide sequence of SEQ ID NO: 43 (herein termed GT3);
(iv) (a): a nucleotide sequence encoding a polypeptide having polymerase activity and having at least 95% identity with the amino acid sequence encoded by the nucleotide sequence of SEQ ID NO: 163 (herein termed wzy);
(b): a nucleotide sequence encoding a polypeptide having polysaccharide transporter activity and having at least 95% identity with the amino acid sequence encoded by the nucleotide sequence of SEQ ID NO: 169 (herein termed wzx); and
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 161 (herein termed GT1);
(c2): a nucleotide sequence having at least 95% identity with the amino acid sequence encoded by the nucleotide sequence of SEQ ID NO: 165 (herein termed GT2);
(c3): a nucleotide sequence having at least 95% identity with the amino acid sequence encoded by the nucleotide sequence of SEQ ID NO: 167 (herein termed GT3); and
(c4) a nucleotide sequence having at least 95% identity with the amino acid sequence encoded by the nucleotide sequence of SEQ ID NO: 181 (herein termed GT4);
(v) (a): a nucleotide sequence encoding a polypeptide having polymerase activity and having at least 95% identity with the amino acid sequence encoded by nucleotides 13939-15042 of SEQ ID NO: 224 (herein termed wzy);
and
(c): a nucleotide sequence encoding a polypeptide having glycosyltransferase (GT) activity comprising:
(c1): a nucleotide sequence having at least 95% identity with the amino acid sequence encoded by nucleotides 4174-4824 of SEQ ID NO: 224 (herein termed GT1);
(c2): a nucleotide sequence having at least 95% identity with the amino acid sequence encoded by nucleotides 11042-12391 of SEQ ID NO: 224 (herein termed GT2);
(c3): a nucleotide sequence having at least 95% identity with the amino acid sequence encoded by nucleotides 13008-13934 of SEQ ID NO: 224 (herein termed GT3); and
(c4): a nucleotide sequence having at least 95% identity with the amino acid sequence encoded by nucleotides 18527-19507 of SEQ ID NO: 224 (herein termed GT4);
(vi) (a): a nucleotide sequence encoding a polypeptide having polymerase activity and having at least 95% identity with the amino acid sequence encoded by the nucleotide sequence of SEQ ID NO: 67 (herein termed wzy);
(b): a nucleotide sequence encoding a polypeptide having polysaccharide transporter activity and having at least 95% identity with the amino acid sequence encoded by the nucleotide sequence of SEQ ID NO: 73 (herein termed wzx);
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 65 (herein termed GT1);
(c2): a nucleotide sequence having at least 95% identity with the amino acid sequence encoded by the nucleotide sequence of SEQ ID NO: 69 (herein termed GT2); and
(c3): a nucleotide sequence having at least 95% identity with the amino acid sequence encoded by the nucleotide sequence of SEQ ID NO: 71 (herein termed GT3);
(c4): a nucleotide sequence having at least 95% identity with the amino acid sequence encoded by the nucleotide sequence of SEQ ID NO: 85 (herein termed GT4);
(c5): a nucleotide sequence having at least 95% identity with the amino acid sequence encoded by the nucleotide sequence of SEQ ID NO: 87 (herein termed GT5); and
(c6): a nucleotide sequence having at least 95% identity with the amino acid sequence encoded by the nucleotide sequence of SEQ ID NO: 89 (herein termed GT6);
(vii) (a): a nucleotide sequence encoding a polypeptide having polymerase activity and having at least 95% identity with the amino acid sequence encoded by nucleotides 5833-6927 of SEQ ID NO: 244 (herein termed wzy);
and
(c): a nucleotide sequence encoding a polypeptide having glycosyltransferase (GT) activity comprising:
(c1): a nucleotide sequence having at least 95% identity with the amino acid sequence encoded by nucleotides 4617-5123 of SEQ ID NO: 244 (herein termed GT1); and
(c2): a nucleotide sequence having at least 95% identity with the amino acid sequence encoded by nucleotides 5120-5827 of SEQ ID NO: 244 (herein termed GT2);
(viii) (a): a nucleotide sequence encoding a polypeptide having polymerase activity and having at least 95% identity with the amino acid sequence encoded by the nucleotide sequence of SEQ ID NO: 123 (herein termed wzy);
(b): a nucleotide sequence encoding a polypeptide having polysaccharide transporter activity and having at least 95% identity with the amino acid sequence encoded by the nucleotide sequence of SEQ ID NO: 129 (herein termed wzx);
(c): a nucleotide sequence encoding a polypeptide having glycosyltransferase (GT) activity comprising:
(c1): a nucleotide sequence having at least 95% identity with the amino acid sequence encoded by the nucleotide sequence of SEQ ID NO: 121 (herein termed GT1);
(c2): a nucleotide sequence having at least 95% identity with the amino acid sequence encoded by the nucleotide sequence of SEQ ID NO: 125 (herein termed GT2); and
(c3): a nucleotide sequence having at least 95% identity with the amino acid sequence encoded by the nucleotide sequence of SEQ ID NO: 127 (herein termed GT3);
(c4): a nucleotide sequence having at least 95% identity with the amino acid sequence encoded by the nucleotide sequence of SEQ ID NO: 143 (herein termed GT4);
(c5): a nucleotide sequence having at least 95% identity with the amino acid sequence encoded by the nucleotide sequence of SEQ ID NO: 145 (herein termed GT5); and
(c6): a nucleotide sequence having at least 95% identity with the amino acid sequence encoded by the nucleotide sequence of SEQ ID NO: 147 (herein termed GT6);
(ix) (a): a nucleotide sequence encoding a polypeptide having polymerase activity and having at least 95% identity with the amino acid sequence encoded by nucleotides 11201-12349 of the complementary strand of SEQ ID NO: 257 (herein termed wzy);
(b): a nucleotide sequence encoding a polypeptide having polysaccharide transporter activity and having at least 95% identity with the amino acid sequence encoded by nucleotides 15538-16953 of the complementary strand of SEQ ID NO: 257 (herein termed wzx); and
(c): a nucleotide sequence encoding a polypeptide having glycosyltransferase (GT) activity comprising:
(c1): a nucleotide sequence having at least 95% identity with the amino acid sequence encoded by nucleotides 9726-10673 of the complementary strand of SEQ ID NO: 257 (herein termed GT1);
(c2): a nucleotide sequence having at least 95% identity with the amino acid sequence encoded by nucleotides 12336-13421 of the complementary strand of SEQ ID NO: 257 (herein termed GT2); and
(c3): a nucleotide sequence having at least 95% identity with the amino acid sequence encoded by nucleotides 13418-14260 of the complementary strand of SEQ ID NO: 257 (herein termed GT3);
(x) (a): a nucleotide sequence encoding a polypeptide having polymerase activity and having at least 95% identity with the amino acid sequence encoded by nucleotides 10707-11846 of the complementary strand of SEQ ID NO: 274 (herein termed wzy);
(b): a nucleotide sequence encoding a polypeptide having polysaccharide transporter activity and having at least 95% identity with the amino acid sequence encoded by nucleotides 15037-16476 of the complementary strand of SEQ ID NO: 274 (herein termed wzx); and
(c): a nucleotide sequence encoding a polypeptide having glycosyltransferase (GT) activity comprising:
(c1): a nucleotide sequence having at least 95% identity with the amino acid sequence encoded by nucleotides 9232-10179 of the complementary strand of SEQ ID NO: 274 (herein termed GT1);
(c2): a nucleotide sequence having at least 95% identity with the amino acid sequence encoded by nucleotides 11833-12918 of the complementary strand of SEQ ID NO: 274 (herein termed GT2); and
(c3): a nucleotide sequence having at least 95% identity with the amino acid sequence encoded by nucleotides 12915-13757 of the complementary strand of SEQ ID NO: 274 (herein termed GT3).

19. A strain of Lactococcus lactis subsp. lactis deposited at DSMZ under accession number DSM 33192.

20. The composition according to claim 9, wherein the one or more further lactic acid bacterium strain(s) comprises a Lactococcus lactis strain comprising an active eps gene cluster capable of producing exopolysaccharide (EPS), wherein the eps gene cluster comprises SEQ ID NO.: 290.

21. A method of increasing viscosity of a fermented milk product, comprising fermenting a milk substrate with one or more strains selected from Lactococcus lactis subsp. cremoris strain DSM 25485 and Lactococcus lactis subsp. lactis strain DSM 33192.

22. A food product comprising a composition according to claim 9.
